(19) United States
(12) Patent Application Publication
(10) Pub. No.: US 2015/0246137 A1
(43) Pub. Date: Sep. 3, 2015

(54) LIPID COATED NANOPARTICLES CONTAINING AGENTS HAVING LOW AQUEOUS AND LIPID SOLUBILITIES AND METHODS THEREOF

(71) Applicant: THE UNIVERSITY OF NORTH CAROLINA AT CHAPEL HILL, Chapel Hill, NC (US)

(72) Inventors: Shutao Guo, Jamaica Plain, MA (US); Leaf Huang, Durham, NC (US); Srinivas Ramishetti, Chapel Hill, NC (US)

(21) Appl. No.: 14/431,516

(22) PCT Filed: Sep. 26, 2013
(86) PCT No.: PCT/US2013/061985

§ 371 Date: Mar. 26, 2015

Related U.S. Application Data
(60) Provisional application No. 61/706,454, filed on Sep. 27, 2012.

(51) Int. Cl. A61K 47/48; A61K 9/51; A61K 31/7048; A61K 9/127; A61K 33/24

(52) U.S. Cl. CPC A61K 47/48815; A61K 9/1271; A61K 33/24; A61K 31/7048; A61K 9/5123

(57) ABSTRACT

Provided herein are compositions that include delivery system complexes comprising a nano-precipitated bioactive compound, wherein the nano-precipitate is encapsulated by a liposome or has at least a portion of its surface coated with a liposome. Because the liposomes contain nano-precipitates of bioactive compounds, the liposomes are capable of utilization in formulating essentially insoluble forms of bioactive agents. Also provided herein are methods for the treatment of a disease or an unwanted condition in a subject, wherein the methods comprise administering the delivery system complexes.

### FIELD OF THE INVENTION

The present invention involves the delivery of low-solubility bioactive compounds using lipid-comprising delivery system complexes.

### BACKGROUND OF THE INVENTION

Agents, such as drugs, that have low-solubility are notoriously difficult to formulate. In particular, the low solubility has precluded the use of the active agents in liposomes. This is because the loading of the active in the liposome is very difficult or the amount of loading is very small or both. As an example, cisplatin [cis-diaminedichloroplatinum(II), CDDP] is a first-line chemotherapy drug widely used for the treatment of many human malignancies. However, the performance of the drug is greatly compromised by its nephro and neuro toxicities. Existing nanoformulations suffer from low loading efficiency and burst drug release kinetics particularly when used with a drug having low solubility. Also, in some instances, the toxicity of highly soluble drugs can severely hinder their practical usage.

Considering the great potential of these essentially insoluble active agents and highly soluble, yet toxic active agents, the need exists for the development of stable vehicles that are able to effectively and safely deliver these therapeutics.

### BRIEF SUMMARY OF THE INVENTION

Provided herein are compositions that include delivery system complexes comprising a nano-precipitated bioactive compound, wherein the precipitate is encapsulated by a liposome or has at least a portion of its surface coated with a liposome. Because the liposomes contain nano-precipitates of bioactive compounds, the liposomes are capable of formulating essentially insoluble forms of bioactive agents. Also provided herein are methods for the treatment of a disease or an unwanted condition in a subject, wherein the methods comprise administering the delivery system complexes. The delivery system complexes can comprise any type of nano-precipitated bioactive compound, including but not limited to, polynucleotides, polypeptides, and drugs.

The delivery system complexes can be used to deliver bioactive compounds to cells. Therefore, provided herein are methods for delivering a bioactive compound to a cell, wherein the method comprises contacting a cell with a delivery system complex comprising a liposome-encapsulated nano-precipitated bioactive compound.

Further, methods are provided for the treatment of diseases or unwanted conditions in a subject, wherein the method comprises administering a delivery system complex comprising a liposome-encapsulated nano-precipitated bioactive compound.

Delivery system complexes can comprise a targeting ligand and are referred to as targeted delivery system complexes. These targeted delivery system complexes can specifically target the bioactive compound to diseased cells, enhancing the effectiveness and minimizing the toxicity of the delivery system complexes.

Further provided herein are methods for making the delivery system complexes.

These and other aspects of the invention are disclosed in more detail in the description of the invention given below.

### BRIEF DESCRIPTION OF THE FIGURES

FIG. 1 shows transmission electron microscopy (TEM) images of LPC core (˜15 nm, A), LPB core (˜25 nm, B), LPI core (˜25 nm, C). Images of LPC (˜30 nm, D), LPB (-4˜nm, E) and LPI (˜40 nm, F) are stained by uranyl acetate. Scale bar: 50 nm.

FIG. 2 depicts A) Effects of Cisplatin (CDDP), LPC and LPI on growth of 1205Lu human melanoma xenografts. B) Effects of cisplatin, LPC and LPI on body weight of mice bearing 1205Lu xenograft. Bodyweight and tumor volume were measured at the indicated time points. A dose of 2.0 mg/kg (with respect to Pt) or PBS was administered weekly by I. V. injection. The red arrows indicate the day of injection. Tumor volume was calculated by TV=(L*W*W)/2, with W being smaller than L. PBS, CDDP and LPI groups contained 5 mice; LPC group contained 7 mice.

FIG. 3 depicts in vitro release kinetics of encapsulated platinum in HEPES buffer at 37° C. from LPC and LPI.

FIG. 4 shows cell uptake of LPC in 1205Lu cells imaged by confocal microscopy. The LPC was labeled as follows: A) NBD-PE lipid (Green); B) Lysosomes (red) and C) nuclear (blue) were stained by Lysotracker-Red and Hoechst 33342, respectively.

FIG. 5 depicts Pt distribution in mouse organs dose of LPC and LPI at dose of 2.0 mg/kg Pt content. The data were collected after 4 h of I. V. Injection. Each group had 3 mice.

FIG. 6 depicts TUNEL assay of A375M human melanoma xenograft 7 days after two weekly injections of LPC at the Pt dose of 3.0 mg/kg.

FIG. 7 depicts 5×10⁶ A375M cells were inoculated in nude mice. Mice were randomly divided into 4 groups (PBS, CDDP, LPC and LPI). Each group had 4-6 mice. And a dose of 1.0 mg/kg Pt was administered by I.V. injection. A) Tumor volume; B) Relative body weight increase.

FIG. 8 depicts 5×10⁶ Vem-resistant 1205Lu cells and 2.5×10⁶ Vem-sensitive 1205Lu cells were inoculated in nude mice. After 12 daily Vem injections, mice were randomly divided into 4 groups. Each group had 4-6 mice. And 2.0 mg/Kg of Pt was administered by I.V. injection for Vem+CDDP and Vem+LPC groups; 100 mg/Kg Vem was administered daily by I.P. injection to Vem, Vem+CDDP and Vem+LPC groups. A) Relative tumor volume of Vem-resistant tumors; B) Relative tumor volume of Vem-sensitive tumors; C) Relative body weight increase.

FIG. 9 shows H&E staining of kidney from the mice received 4 doses treatments. Yellow symbols indicate signs of nephrotoxicity.

FIG. 10 depicts bioactive compounds and cations that can form bioactive compounds that are nano-precipitates of salts of the bioactive compounds. Note that both anti-cancer and anti-viral drugs can be utilized.

FIG. 11 shows a transmission electron microscopy photograph of nanoparticles prepared by mixing etoposide phosphate with InCl₃. The nanoparticles were about 30 nm in diameter and were stabilized with a lipid membrane.

FIG. 12 shows characterizations of IEP core nanoparticles: A) TEM image; B) EDS spectrum; C) UV/VIS absorption spectrum; D) ESI-MS for EP encapsulated nanoparticles.

FIG. 13 depicts cellular uptake of labeled IEP nanoparticles in H460 Cells; outer lipid layer is labeled with DiI (red) and inner core labeled with DOPA-NBD (green) and nucleus staining with DAPI (blue): HP-haloperidol.

FIG. 14 shows in vitro toxicity and mechanistic studies of IEP nanoparticles in H460 treated cells; A) MTT assay; B) Caspase assay; C) Western blot assay; D) Flow cytometer analysis of cell cycle arrest.

FIG. 15 shows in vivo therapeutic effect of IEP nanoparticles in H460 xenograft mouse tumor model; A) Tumor growth inhibition; B) Body weight change (n=5. *p<0.005; PBS vs. In/EP-PEG AA); arrows indicating the injection schedule.

FIG. 16A shows IEP nanoparticles triggered the tumor cell apoptosis and inhibit the cell proliferation: Tunel assay (upper panel) and PCNA analysis (lower panel); B) Tunel assay quantification *p<0.002; PBS vs. In/EP-PEG; **p<0.005; PBS vs. In/EP-PEG AA; C) PCNA *p<0.005; PBS vs. In/EP-PEG; PBS vs. In/EP-PEG AA.

FIG. 17 depicts the safety studies: H&E analysis of mouse major organs treated with IEP nanoparticles.

FIG. 18 Shows dynamic light scattering (DLS) analysis of nanoparticles: A) size for IEP-PEG; B) zeta potential for IEP-PEG; C) size for IEP-PEG AA; D) zeta potential for IEP-PEG AA.

FIG. 19 depicts in vivo distribution studies of IEP nanoparticles.

FIG. 20 shows NPs were characterized by size and dispersity; (A) Characterization of LPC NPs using TEM. LPC NPs were negatively stained with uranyl acetate. Scale bar represents 50 nm; (B) Characterization of LPC NPs using dynamic light scattering (DLS).

FIG. 21 depicts LPC NPs exhibited high toxicity and effective transport ability of CDDP; (A) IC₅₀ of CDDP and LPC NPs in A375M cells; (B) The amount of cell uptake of CDDP and LPC NPs in A375M cells quantified using ICP-MS. Data is expressed as % uptake; (C) The amount of the Pt drug associated with cells after incubation with 100 μM CDDP or LPC NPs in 24 well plates. Each bar represents the mean±SEM of 3 independent experiments. The analysis of variance is completed using a one-way ANOVA.

FIG. 22 shows LPC NPs showed high accumulation in A375M tumor cells and impeded the growth of tumors at 1.0 mg/kg of Pt; (A) Pt distribution in A375M tumor bearing mice administered with CDDP and LPC NPs. One mg/kg of Pt was administered weekly via IV injection; (B-C) effects of CDDP and LPC NPs on tumor growth and body weight respectively of A375M tumor bearing mice. The arrows indicate the time of injection. The results are displayed as mean ±SEM (error bars) of five animals per group. The analysis of variance is computed using a one-way ANOVA. ** indicates p<0.01.

FIG. 23 shows LPC NPs induced apoptosis in 90% of tumor cells; Effects of LPC NPs on A375M tumor cell apoptosis is analyzed using TUNEL assay; The tumors were treated once a week for two weeks with IV injections containing 3.0 mg/kg of Pt.

FIG. 24 shows neighboring effect studied using TUNEL assay and detection of CDDP-DNA adduct; LPC NPs were labeled with DiI dye (red). The mice were sacrificed twenty-four hours after receiving a single IV injection of LPC NPs at a dose of 1.0 mg/kg Pt; (A) The distribution of NPs was tracked by DiI dye, and the apoptotic tumor cells were detected by the TUNEL assay; (B) the formation of CDDP-DNA in tumor cells detected by CDDP-DNA antibody; (C) The number of TUNEL positive cells measured as a function of the distance to its nearest DiI positive cell; (D) the number of CDDP-DNA adduct positive cells measured as a function of the distance to its nearest DiI positive cell.

FIG. 25 shows the procedure used to validate the neighboring effect in vitro.

FIG. 26 depicts that LPC NPs showed a controlled release pattern in medium and in cells; (A) In vitro release kinetics of encapsulated platinum in 50% FBS medium at 37° C. and the cellular release of Pt from LPC NPs treated cells; (B) Percentages of Pt in the released medium that were pelletable (green) and unpelletable (red) are shown; (C) The cytotoxic activity of released drugs from NP treated cells at different time points. Cells were treated with 5 μM CDDP for comparison. Each bar represents the mean±SEM of 3 independent experiments.

FIG. 27 shows the neighboring effect demonstrated by co-culturing CDDP transfected A375M-GFP cells and A375M cells at a 1:10 ratio. A375M-GFP cells were treated with LPC NPs (50 μM) for 4 h. After 24 or 48 h of co-culturing, the cell nuclei were stained with Hoechst 33342 (blue); (A-B) Apoptotic cells were stained with Alexa Fluor 568-labeled Annexin V (red) for fluorescence microscopy and flow cytometry analysis.

FIG. 28 shows HE staining showing LPC NPs did not induce nephrotoxicity. H&E staining of liver, spleen and kidney tissue from mice that received four doses of treatment (1 mg/kg each).

FIG. 29 shows Dil-labeled LPC NPs (red) in liver were mainly taken up by

Kupffer cells. Kupffer cells were stained using CD68 antibody (green) and the hepatocyte nuclei were stained using DAPI (blue). The mice were sacrificed twenty-four hours after receiving a single IV injection of LPC NPs at a dose of 1.0 mg/kg Pt.

FIG. 30 shows that although CDDP-DNA adducts were detected in kidney, liver and spleen, no neighboring effect is observed. The distribution of DiI-labeled LPC NPs (red) and the detection of CDDP-DNA adduct (green) in kidney, liver and spleen. The mice were sacrificed twenty-four hours after receiving a single IV injection of LPC NPs at a dose of 1.0 mg/kg Pt.

FIG. 31 shows that no significant apoptosis was detected in organs from LPC NPs treated mice. The detection of apoptotic cells in heart, liver, spleen, lung and kidney using TUNEL assay. The mice were sacrificed twenty-four hours after receiving a single IV injection of LPC NPs at a dose of 1.0 mg/kg Pt. Apoptotic cells were detected using TUNEL assay (green) and the cell nuclei were stained using DAPI (blue).

FIG. 32 shows In vitro cell uptake of LPC NPs imaged using confocal microscopy. LPC NPs were labeled with NBD-PE lipid (green). Lysosome (red) and nucleus (blue) were stained by Lysotracker-Red and Hoechst 33342, separately.

FIG. 33 shows Kidney and liver function parameters, AST (aspartate aminotransferase), ALT (alanine aminotransferase) and BUN (blood urea nitrogen).

FIG. 34 shows H&E staining of heart and lung from mice which received four doses of treatment (1 mg/kg each).

FIG. 35 shows TEM images of DOPA-coated, CDDP NPs prepared using different surfactant systems. The surfactants used to create the microemulsion are a mixture of Igepal-520 system (Igepal-520: cyclohexane=30:70 (v/v)) and Triton X-100 system (Triton X-100: Hexanol: cyclohexane=15:10:75 (v/v/v); the volume ratio of Igepal-520 system to Triton X-100 system is 6:2 (A), 2:6 (B) and 0:8 (C) respectively.

FIG. 36 shows the XPS study of Pt 4f (A), N is (B), Cl 2p (C) and P 2p (D) on CDDP and DOPA-coated CDDP NPs.

FIG. 37 shows the ¹H NMR spectra of CDDP and DOPA-coated CDDP NPs in DMF-d7.

FIG. 38 shows the TEM image of CDDP NPs negatively stained using uranyl acetate (A) and size distribution by dynamic light scattering (B) of LPC NPs.

FIG. 39 shows the cell uptake of LPC NPs in 1205Lu cell line imaged by confocal microscopy. The LPC NPs were labeled with NBD-PE lipid (green). Lysosome (red) and nucleus (blue) were stained by Lysotracker-Red and Hoechst 33342, separately.

FIG. 40 shows the cytotoxicity of free CDDP and LPC NPs in 1205Lu cells (A) and the amount of cell uptake of CDDP and LPC NPs in 1205Lu tumor cells quantified using ICP-MS (B). Data is expressed as the amount of the Pt drug associated with cells incubated with 100 tM CDDP or LPC NPs in 24 well plates. Each bar represents the mean±SEM of three independent experiments. The analysis of variance was completed using a one-way ANOVA.

FIG. 41 shows the effects of free CDDP and LPC NPs on growth of 1205Lu tumors (A) and relative body weight (B). Free CDDP and LPC NPs were administered intravenously at a dose of 2.0 mg/kg Pt. After mice were sacrificed, tumor tissue was sectioned for TUNEL assay (C). The arrows indicate the time of injection. Tumor volume (TV) was calculated using the following formula: TV=(L×W²)/2, with W<L. The results are shown as means±SEM (error bars) of 5 mice per group and are representative of two independent experiments. The analysis of variance is completed using a one-way ANOVA. *p<0.05; **p<0.01.

### DETAILED DESCRIPTION OF THE INVENTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Provided herein are delivery system complexes comprising a nano-precipitated bioactive compound, wherein the nano-precipitated compound is encapsulated or coated on at least a portion thereof by a liposome. The low solubility of bioactive compounds, such as CDDP is often a problem in formulating the compounds, but it is a unique advantage in the formulations described herein. Unlike existing technologies that are less efficient as the solubility of an agent decreases, the subject matter described herein advantageously utilizes low solubility of a bioactive compound nano-precipitate in oil and water to prepare a delivery system complex.

As an example, the last step in the classical synthesis of CDDP proceeds by addition of KCl to a highly soluble precursor, cis-diaminedihydroplatinum(II) (Scheme 2). CDDP precipitates out as the bulk product of the reaction. As described herein, advantageously, a nano-precipitate of CDDP was prepared by mixing two reverse micro-emulsions containing reactants. In this way, a nano-precipitate of CDDP was formed and coated with a single lipid bilayer coating. The external layer can comprise a PEGylated lipid. The final nanoparticles (NPs) can also comprise anisamide which binds with the sigma receptor over-expressed in many epithelial cancer cells. These NPs with CDDP and its analogs containing bromide and iodide replacing chloride have been prepared. The resulting NPs are called Lipid/Pt/Cl (LPC), Lipid/Pt/Br (LPB) and Lipid/Pt/I (LPI), respectively. Up to now, the bromide and iodide analogs would not have been considered viable bioactive compound candidates because of very low solubility. By converting non-candidates into potential bioactive compounds that can be successfully formulated for delivery, the subject matter described herein makes a significant contribution to medicine, in particular, cancer chemotherapy.

These NPs described herein are very stable and showed slow drug release kinetics without burst release. The half-life (t_{1/2}) for drug release of LPC and LPI is 45 and 80 h, respectively. Human melanoma cells take up a large amount of LPC and stay alive until 48-72 h later. In contrast, cells treated with same concentration of free CDDP became apoptotic in 4 h. The results indicated a slow and sustained drug release occurred inside the cancer cells. More importantly, both LPC and LPI showed potent anti-cancer activity in two human melanoma xenograft models at 2-3 mg/kg weekly dosing schedule without any kidney or liver toxicities. Additionally, drug loading is particularly high in the NPs that have been prepared and particle stability can be optimized. Since LPB and LPI are slow releasing formulations, they can show activity with an infrequent and low dosing schedule for slow growing tumors which is a common feature of human malignancy.

Further provided are delivery system complexes comprising a nano-precipitated bioactive compound surrounded by a lipid bilayer. In embodiments, the nano-precipitate can be ionically bound to the inner leaflet of the lipid bilayer. Methods for making the delivery system complexes as well as methods for the use of the complexes are further provided herein. The delivery system complexes can be used to deliver low-solubility or essentially insoluble bioactive compounds to cells and to treat diseases or unwanted conditions in those embodiments wherein the bioactive compound comprised within the delivery system complex has therapeutic activity against the disease or unwanted condition.

As used herein, a “delivery system complex” or “delivery system” refers to a complex comprising a bioactive compound and a means for delivering the bioactive compound to a cell, physiological site, or tissue.

As used herein the term “nano-precipitate” refers to a nano-precipitated bioactive compound or precursor thereof that has low-solubility in water and oil or is essentially insoluble in water and oil, and a lipid encapsulating or coating at least a portion of the surface of the bioactive compound. The term “low-solubility” means that the nano-precipitated bioactive compound or precursor thereof is not solubilized in water and oil to an appreciable amount. As used herein, the bioactive compound is prepared as a nano-precipitate by contacting the compound or a precursor of the compound with a species that forms a nano-precipitate of the bioactive compound. As used herein, the nano-precipitated bioactive compound has a lipid coating as described elsewhere herein. Thus, a nano-precipitate is distinguishable from bulk precipitates. Additionally, bulk precipitates do not have nano-sized lipid coated particles. Utilizing the methods disclosed herein, bioactive compounds can be prepared as nano-precipitates and formulated in delivery system complexes.

Cis-diaminedichloroplatinum(II) (CDDP or cisplatin), a widely used anti-cancer drug, has many side-effects including nephro and neuro toxicities (4-6). Newer generations of Pt drugs (such as carboplatin and oxaliplatin), although less toxic, are also less effective. CDDP has a low solubility which limits the development of novel formulations. Lipid coated calcium phosphate (LCP) nanoparticles have been prepared (7-12) with the size of 30-40 nm in diameter through a nano-precipitation method in micro-emulsion. However, the core of those particles contains a precipitate comprising a core precipitate, i.e., seed, in addition to the bioactive compound. Additionally, the bioactive compound is not a precipitate itself and the precipitate seed material itself in those particles is not considered the bioactive compound. Moreover, the nanoparticles described herein can contain bioactive compounds that are essentially insoluble or bioactive compounds that can be precipitated using an ion.

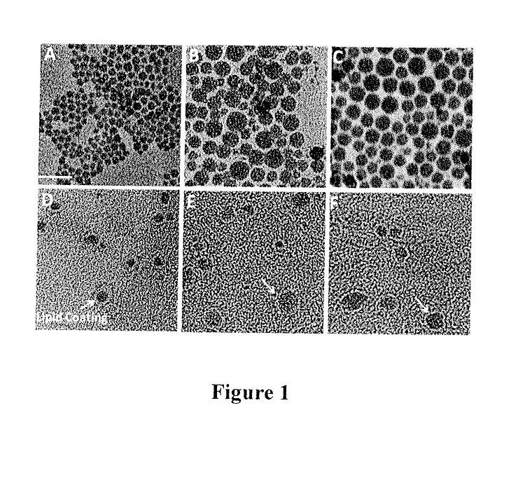

Advantageously, the low solubility of CDDP was utilized to prepare a nano-precipitate. This nano-precipitate has a lipid bilayer coat (FIG. 1). This coating also stabilized the nano-precipitate. Moving from chloride to bromide and to iodide, the platinum compound becomes progressively less soluble (15). However, with the method of encapsulation disclosed herein, these halide complexes of cis-Pt(II) can show excellent colloidal stability and anti-cancer activity.

Although it is reported CDDP has been successfully encapsulated into liposomes and some formulations were investigated or are being evaluated in clinical trials, there is no FDA approved formulation (16,17). Additionally, the loading of the liposomes with drug is much lower than the present technology can achieve. Completed experiments indicate that a micro-emulsion method (Scheme 1) can be utilized to encapsulate CDDP nano-precipitate in a single lipid bilayer vesicle. The resulting drug formulations are called Lipid/Pt/Chloride (LPC), Lipid/Pt/Bromide (LPB) and Lipid/Pt/Iodide (LPI), which contain CDDP, cis-diaminedibromoplatinum(II) and cis-diaminediiodoplatinum(II), respectively. Because the nano-precipitates described herein do not require seeding material to form a nano-precipitate, the amount of loading of the bioactive compound in the liposome is substantially greater than achievable with existing technology.

Importantly, the unformulated Pt compounds containing either Br or I are not anti-cancer drugs due to their very low solubility. Employing the methods described herein, both LPB and LPI can be effective anti-cancer drugs which can be systemically delivered to the tumor cells.

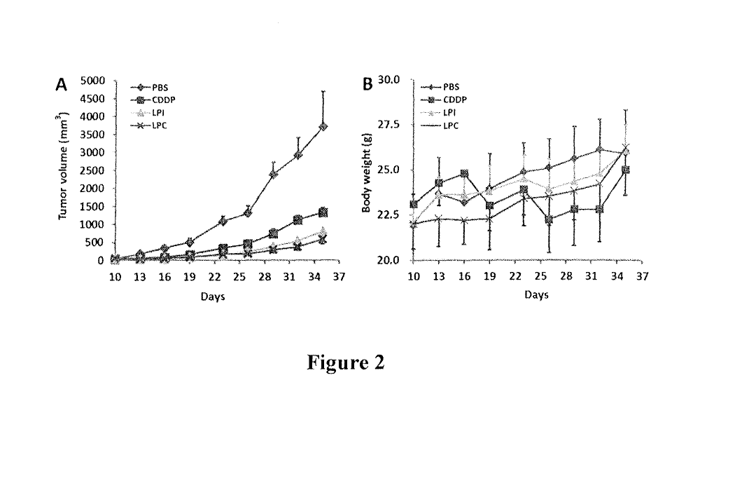

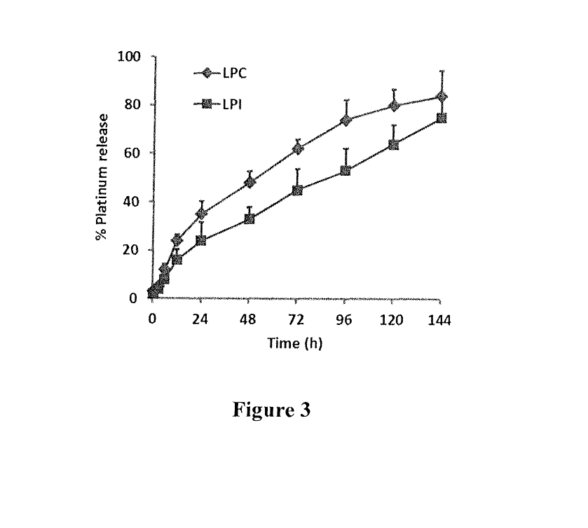

Another drawback of the current liposomal CDDP formulations is their relatively large size (100 to 200 nm). The nanoparticles described herein are much smaller (FIG. 1) but with a high drug loading capacity. Data presented elsewhere herein indicate that both LPC and LPI were equally active in inhibiting the growth of human melanoma tumors in xenograft models (FIG. 2A). Since the drug release rate of LPI is approximately two times as slower than that of LPC (FIG. 3), slow releasing formulations, such as LPI, might be suitable for slow growing tumors which are characteristic of many human malignancies. Data shown elsewhere herein indicated that about 90% of the tumor cells underwent apoptosis 7 days after dosing of LPC, suggesting that drug released from cells taken up the NPs can kill the neighboring cells.

Nanoparticles, through both passive and active targeting, can enhance the intracellular concentration of drugs in cancer cells while avoiding toxicity in normal cells. PEGylated liposome-based nanoparticles can efficiently deliver nucleic acid, chemo-drugs and proteins to the solid tumors and metastatic sites. Nanoparticles significantly increase the local drug accumulation, particularly in the tumor, by evasion from RES uptake and enhanced permeability and retention (EPR) (18). This approach drastically lowers the effective therapeutic dose and minimizes the undesired side effects after systemic drug administration. However, there still must be sufficient loading of the bioactive, which is difficult particularly for essentially insoluble drugs. The accumulation of nanoparticles in the tumor site depends highly on the leakiness of tumor vasculature. It is desired to design and manufacture small nanoparticles less than 50 nm in diameter to penetrate not-so-leaky tumors (19). The Pt formulations disclosed herein are <40 nm in diameter, making them particularly suitable for tumor delivery (FIG. 1).

Furthermore, in embodiments, the nanoparticle formulations were modified with an anisamide (AA) as ligand for targeted delivery. The role of the ligand is to convey a rapid endocytosis of the bound nanoparticles.

Liposomal and polymeric formulations of CDDP all suffer from relatively low loading efficiency or stability (20-25) (Table 1) compared to the nanoparticles disclosed herein.

TABLE 1
CDDP loading of liposomal and polymeric formulations

| Formulation | CDDP Drug loading | Ref |
|---|---|---|
| SPI-77 | 1.38 wt % | (1) |
| Nanoplatin | 39.0 wt % | (2) |
| SACNs | 11.7 wt % | (3) |
| Pt-PLGA-b-PEG-Apt-NPs | 2.83 wt % | (13) |
| Lipoplatin | 8.90 wt % | (14) |
| LPC | 47.1 wt % | Current work |

One of the liposomal formulations, SPI-77, has failed a phase II clinical trial due to insufficient release of the drug from the liposomes (16,26,27). A block co-polymer formulation, Nanoplatin, has relatively higher drug loading and stability than other formulations in Table 1. (28,29). However, it is a polymer-based delivery system. CDDP complexed with cholesterol hemisuccinate derivative and formulated in a micellar assembly (SACNs) showed reduced nephrotoxicity and enhanced anti-tumor activity (30). A prodrug of CDDP with Pt(IV) formulated in nanoparticles has shown anti-tumor activity (13). However, the conversion of Pt(IV) prodrug to CDDP requires a reducing environment which could be variable in different tumors. The nano Pt formulations disclosed herein do not require any conversion conditions.

Melanoma was the fifth-most diagnosed cancer in 2011, with over 70,000 new cases and nearly 9,000 deaths (31). Although CDDP is one of the most common anti-neoplastic agents for melanoma in clinical trials (32), cancer chemotherapy efficacy is frequently impaired by either intrinsic or acquired tumor resistance, a phenomenon termed multi-drug resistance (MDR) (33-35). MDR may result from several mechanisms, such as alterations impairment of tumor apoptotic pathways (36,37), repair of damage cellular targets (38,39) and particularly reduced drug accumulation in tumor cells (40-45).

The accumulation of nanoparticulate drug formulation with long blood circulation in tumor is much higher than the free drug. And nanoparticles containing a targeting ligand such as anisamide and TAT peptide can be internalized efficiently by tumor cells and penetrate into cell nucleus. Targeted therapeutic NPs have emerged as an alternative over conventional small molecule chemotherapeutics aimed at specifically targeting the therapeutic payload to tumors and overcoming multiple drug resistance (46-49). However, again, there is a requirement that there must be sufficient drug loading of the particle. Low levels drug loading is a major obstacle with known technology.

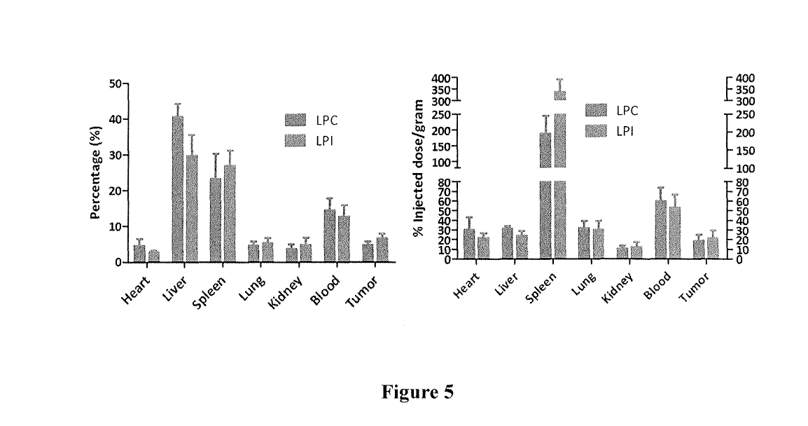

The advent of selective drug delivery using molecular targets against melanoma has shown promise, as several small-molecule drugs are in late-stage clinical trials or have already been approved by the FDA. One such approved drug, Vemurafenib (Zelboraf®), is an inhibitor of B-Raf^{V600E} kinase. The mutation, found in 50-70% of malignant melanomas, constitutively activates the MAPK pathway (50). Administration of this drug has shown marked tumor reduction even in patients in the latest-stage of the diseases (51,52). Unfortunately, the development of drug resistance leads to the failure of treatments with Vemurafenib (53). The ability of melanomas to form resistance to Vemurafenib and other drugs has led to attempts to find a therapeutic combination that will inhibit the tumor growth. Data disclosed in FIG. 5 indicate that human melanomas were very sensitive to LPC and apoptosis was readily induced.

An LCP platform to deliver bioactive molecules, such as functional genes, silencing RNA and chemo-drugs, that are contained among a precipitate core is known. (9,10). In these formulations, an outer layer of a cationic lipid (DOTAP) and high density of PEG was coated on the calcium phosphate cores. The cationic lipid DOTAP allows the nanoparticles to be internalized by tumor cells more efficently and to subsequently escape from the lysosomes. Additionally, a high density of PEGylation can help the nanoparticles avoid RES system, improving drug pharmacokinetics and drug bioavailability. Both components are critical for the successful delivery of drugs into tumors. However, having core material that is other than the bioactive compounds can lead to lower overall percentage of loading of the bioactive.

In contrast, the formulations described herein would be favorable due to high drug loading capacity. The drug loading is calculated by the ratio of Pt_{CDDP}/P_{lipid} determined by ICP-MS; 47.1 wt %. In another aspect, the platform technology described herein can be applicable to the manufacture of many other CDDP analog nanoparticulate formulations. In addition, the platform technology can improve the solubility of platinum based drug candidates with poor solubility, such as cis-diamminedibromoplatinum(II) and cis-diamminediiodoplatinum(II).

Accordingly, in an embodiment, the subject matter described herein is directed to a delivery system complex comprising a bioactive compound, wherein said bioactive compound is a nano-precipitated compound having at least a portion of its surface coated by a liposome or encapsulated by a liposome, wherein said nano-precipitated bioactive compound has low solubility in water and oil and is present in an amount of at least 10% wt of said liposome.

In this embodiment, the nano-precipitated bioactive compound is formed as a salt in a reverse microemulsion that results in the nano-precipitated bioactive compound having at least a portion of its surface coated by a liposome or the nano-precipitate is encapsulated by a liposome, wherein the nano-precipiated bioactive compound has low solubility in water and oil. In embodiments, the nano-precipitate consists essentially of the bioactive compound in its nano-precipitated salt form and a lipid coating. Preferably, the nano-precipitate consists of the bioactive compound in its nano-precipitated salt form and a lipid coating. In some cases, more than one bioactive compound can be co-precipitated by a single ion to form mixed insoluble salts that are nano-precipitates. For example, both etoposide phosphate and gemcitabine phosphate can be nano-precipitated using InCl₃ in the methods described herein. Liposomes containing nano-precipitates of mixed Indium salts of etoposide phosphate and gemcitabine phosphate can therefore be prepared. Different bioactive compounds in the liposome can inhibit the same or different biochemical pathways in the target cells to perform additive or synergistic therapeutic activities.

Importantly, the lack of required seeding material in the nano-precipitate provides for substantially increased loading potential. Loading of the delivery system complex with the nano-precipitate can resulit in an amount of nano-precipitate of at least 10% wt of said liposome. Preferably, the amount is from about 20 to about 70% wt or from about 20% to about 85% wt; from about 30 to about 60%; and more preferably from about 40% wt to about 50% wt. The delivery system complex can further comprise components that are specifically listed elsewhere herein.

In some instances, a bioactive compound can be higly potent, however, its practical applicablity is severely limited by the high toxcity, low bioavailability, instability or the like. Accordingly, some embodiments are directed to liposome encapsulated, nano-precipitated bioactive compound, wherein the bioactive compound is highly soluble yet possesses above-mentioned undesirable properties. In some embodiments, such highly soluble bioactive compounds can be precipitated out of a solution using appropriate metal counter ions. Such metal ions include, but not limited to In⁺³, Gd⁺³, Mg⁺², Zn+2 and Ba⁺². For example, Etoposide, an analog of the anti-cancer agent podophilotoxin, is clinically used for the treatment of small cell lung cancer and testicular cancer, as well as many other cancers (55). The mechanism of its anti-cancer activity involves inhibition of topoisomerase II, an enzyme responsible for DNA strand ligation during cell division. Cancer cells rely on this enzyme to a greater extent than healthy cells because of their rapid growth(56). Etoposide forms a complex with DNA and topoisomerase II and prevents re-ligation of the DNA strand, resulting in strand breakage and subsequent apoptosis. Due to the limited solubility of etoposide, intravenous administration of the drug is challenging and often results in local concentrations insufficient for therapeutic effect. To address this problem, the water soluble prodrug analog, etoposide phosphate, was synthesized (57). Etoposide phosphate (EP) is highly soluble in water and readily metabolized to its parent molecule, etoposide, once intravenously administered. Dephosphorylation converts the pro drug to the active moiety exhibiting anticancer activity (57-59). Although administration of EP resolved the solubility issue and reduced side effects, parenteral administration of EP frequently causes leukopenia and neutropenia in patients. These adverse effects underscore the need for a targeted delivery system to carry EP to the appropriate cells after systemic administration. Over the past few decades, there have been major diagnostic and therapeutic advances in cancer nanomedicine (60). Nanoparticles can extravasate through leaky tumor vasculature and preferentially accumulate in tumor tissue due to enhanced permeability and retention (EPR) effects (61, 62). A number of nanoparticle systems based on liposomes, polymers, inorganic materials etc. have been developed for delivery of anticancer drugs and imaging agents to tumors(63).

Surprisingly and unexpectedly, it was found that indium chloride can co-precipitate with EP. Such an indium-EP complex precipitate can be used as a carrier to target the delivery of EP to tumor cells using embodiments of the present invention.

Previous reports have demonstrated in vitro delivery of etoposide using different nanoparticle formulations, including SWNT modified with EGF (64), strontium carbonate(65), lipid nano capsules (LNC)(66) and other polymer based nanoparticles(67-70). In a recent study, intra-tumoral injection of etoposide encapsulated in poly (ethylene glycol)-co-poly (sebacic acid) (PEG-PSA) polymeric nanoparticles exhibited significant antitumor activity compared to control in an NCI-H82 xenograft mouse model(71). This route of administration has not been established as an alternative in routine clinical practice; however similar results have been reported by others but were based only on work with cultured cells. Indium complexes have been routinely used in solar cells, photo detectors, liquid crystal displays and as a catalyst in chemical reactions (72, 73). To best of our knowledge, this is the first time an indium-based nanoparticle drug delivery system has been reported for EP. A radionuclide of indium (¹¹¹In) is also an excellent) contrast agent for diagnostic imaging by single photon emission computed tomography (SPECT), making the complex a potential theranostic agent (74-77).

In some embodiments, a lipid-stabilized indium-EP complex in nano size is synthesized using a micro emulsion system. In some of the embodiments, the surface of the nanoparticles is heavily PEGylated to increase colloidal stability in circulation and reduce nonspecific uptake by the mononuclear phagocyte system (MPS). In some embodiments, these nanoparticles are also functionalized with anisamide (AA), to target the sigma receptor over expressed on tumor cells to facilitate cellular uptake (75, 76). The in vitro and in vivo performance of these nanoparticles are characterized in terms of tumor-targeted EP delivery. Additionally, systemic toxicity is examined to establish the safety of these nanoparticles.

In some embodiments, the delivery system complex comprises a biodegradable ionic precipitate comprising a bioactive compound and In⁺³, wherein said biodegradable ionic precipitate is encapsulated by a lipid bilayer membrane. In such embodiments, the lipid bilayer comprises a first lipid and a second lipid. In these embodiments, the delivery system complex has any one of the properties of high loading capacity, high bioavailability, less toxicity, higher rate of absorption and improved efficacy.

As used herein, “high loading capacity” means an improved or better loading capacity of the active compound than any of the known liposomal or polymeric formulations of that particular active compound.

As used herein, “high bioavailability” means a better or improved bioavailability of the bioactive compound in comparison to the bioavailability of the free bioactive compound. By “free bioactive compound” is meant a bioactive compound not encapsulated with a lipid bilayer membrane.

As used herein, “less toxicity” means less or not toxic in comparison to the free bioactive compound or any known formulation thereof.

As used herein, “higher rate of absorption” means a better or improved rate of absorption of the active compound in comparison to the free bioactive compound or any known formulation thereof.

As used herein, “improved efficacy” means efficacy of the active compound that is better in kind or degree of both in comparison to any of the known liposomal or polymeric formulations of that particular active compound.

The above properties can be measured and quantified using any of the well-known methods in the art.

I. Liposome-encapsulated Nano-precipitated Bioactive Compounds and Methods of Making the Same

The presently disclosed delivery system complexes comprise a liposome that encapsulates at least a portion of a nano-precipitated bioactive compound. In other words, the bioactive compound(s) is nano-precipitated and is encapsulated or coated on at least a portion of its surface by a lipid to form the nano-precipitate. Methods of preparing a single lipid bilayer are disclosed in WO2011/017297, herein incorporated by reference in its entirety.

Liposomes are self-assembling, substantially spherical vesicles comprising a lipid bilayer that encircles a core, which can be aqueous, wherein the lipid bilayer comprises amphipathic lipids having hydrophilic headgroups and hydrophobic tails, in which the hydrophilic headgroups of the amphipathic lipid molecules are oriented toward the core or surrounding solution, while the hydrophobic tails orient toward the interior of the bilayer. The lipid bilayer structure thereby comprises two opposing monolayers that are referred to as the “inner leaflet” and the “outer leaflet,” wherein the hydrophobic tails are shielded from contact with the surrounding medium. The “inner leaflet” is the monolayer wherein the hydrophilic head groups are oriented toward the core of the liposome. The “outer leaflet” is the monolayer comprising amphipathic lipids, wherein the hydrophilic head groups are oriented towards the outer surface of the liposome. Liposomes typically have a diameter ranging from about 25 nm to about 1 μm. (see, e.g., Shah (ed.) (1998) Micelles, Microemulsions, and Monolayers: Science and Technology, Marcel Dekker; Janoff (ed.) (1998) Liposomes: Rational Design, Marcel Dekker). The term “liposome” encompasses both multilamellar liposomes comprised of anywhere from two to hundreds of concentric lipid bilayers alternating with layers of an aqueous phase and unilamellar vesicles that are comprised of a single lipid bilayer. Methods for making liposomes are well known in the art and are described elsewhere herein.

As used herein, the term “lipid” refers to a member of a group of organic compounds that has lipophilic or amphipathic properties, including, but not limited to, fats, fatty oils, essential oils, waxes, steroids, sterols, phospholipids, glycolipids, sulpholipids, aminolipids, chromolipids (lipochromes), and fatty acids,. The term “lipid” encompasses both naturally occurring and synthetically produced lipids. “Lipophilic” refers to those organic compounds that dissolve in fats, oils, lipids, and non-polar solvents, such as organic solvents. Lipophilic compounds are sparingly soluble or insoluble in water. Thus, lipophilic compounds are hydrophobic. Amphipathic lipids, also referred to herein as “amphiphilic lipids” refer to a lipid molecule having both hydrophilic and hydrophobic characteristics. The hydrophobic group of an amphipathic lipid, as described in more detail immediately herein below, can be a long chain hydrocarbon group. The hydrophilic group of an amphipathic lipid can include a charged group, e.g., an anionic or a cationic group, or a polar, uncharged group. Amphipathic lipids can have multiple hydrophobic groups, multiple hydrophilic groups, and combinations thereof. Because of the presence of both a hydrophobic group and a hydrophilic group, amphipathic lipids can be soluble in water, and to some extent, in organic solvents.

As used herein, “hydrophilic” is a physical property of a molecule that is capable of hydrogen bonding with a water (H₂O) molecule and is soluble in water and other polar solvents. The terms “hydrophilic” and “polar” can be used interchangeably. Hydrophilic characteristics derive from the presence of polar or charged groups, such as carbohydrates, phosphate, carboxylic, sulfato, amino, sulfhydryl, nitro, hydroxy and other like groups.

Conversely, the term “hydrophobic” is a physical property of a molecule that is repelled from a mass of water and can be referred to as “nonpolar,” or “apolar,” all of which are terms that can be used interchangeably with “hydrophobic.” Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s).

Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, dioleoyl phosphatidic acid, and dilinoleoylphosphatidylcholine. Other compounds lacking in phosphorus, such as sphingolipid, glycosphingolipid families, diacylglycerols and β-acyloxyacids, also are within the group designated as amphipathic lipids.

In some embodiments, the liposome or lipid bilayer comprises cationic lipids. As used herein, the term “cationic lipid” encompasses any of a number of lipid species that carry a net positive charge at physiological pH, which can be determined using any method known to one of skill in the art. Such lipids include, but are not limited to, the cationic lipids of formula (I) disclosed in International Application No. PCT/US2009/042476, entitled “Methods and Compositions Comprising Novel Cationic Lipids,” which was filed on May 1, 2009, and is herein incorporated by reference in its entirety. These include, but are not limited to, N-methyl-N-(2-(arginoylamino) ethyl)-N, N-Dioctadecyl aminium chloride or di stearoyl arginyl ammonium chloride] (DSAA), N,N-di-myristoyl-N-methyl-N-2[N′-(N⁶-guanidino-L-lysinyl)]aminoethyl ammonium chloride (DMGLA), N,N-dimyristoyl-N-methyl-N-2[N²-guanidino-L-lysinyl]aminoethyl ammonium chloride, N,N-dimyristoyl-N-methyl-N-2[N′-(N2, N6-di-guanidino-L-lysinyl)]aminoethyl ammonium chloride, and N,N-di-stearoyl-N-methyl-N-2[N′-(N6-guanidino-L-lysinyl)]aminoethyl ammonium chloride (DSGLA). Other non-limiting examples of cationic lipids that can be present in the liposome or lipid bilayer of the presently disclosed delivery system complexes include N,N-dioleyl-N,N-dimethylammonium chloride (“DODAC”); N-(2,3-dioleoyloxy) propyl)-N,N,N-trimethylammonium chloride (“DOTAP”); N-(2,3-dioleyloxy) propyl)-N,N,N-trimethylammonium chloride (“DOTMA”) or other N-(N,N-1-dialkoxy)-alkyl-N,N,N-trisubstituted ammonium surfactants; N,N-distearyl-N,N-dimethylammonium bromide (“DDAB”); 3-(N-(N′,N′-dimethylaminoethane)-carbamoyl) cholesterol (“DC-Chol”) and N-(1,2-dimyristyloxyprop-3-yl)-N,N-dimethyl-N-hydroxyethyl ammonium bromide (“DMRIE”); 1,3-dioleoyl-3-trimethylammonium-propane, N-(1-(2,3-dioleyloxy)propyl)-N-(2-(sperminecarboxamido)ethyl)-N,N-dimethy-1 ammonium trifluoro-acetate (DOSPA); GAP-DLRIE; DMDHP; 3-β[⁴N-(¹N,⁸N-diguanidino spermidine)-carbamoyl] cholesterol (BGSC); 3-β[N,N-diguanidinoethyl-aminoethane)-carbamoyl] cholesterol (BGTC); N,N¹,N²,N³ Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3 dioleoyloxy-1,4-butanediammonium iodide) (Tfx-50); 1,2 dioleoyl-3-(4′-trimethylammonio)butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DL-1,2-dioleoyl-3-dimethylaminopropyl-β-hydroxyethylammonium (DORI) or DL-1,2-O-dioleoyl-3-dimethylaminopropyl-β-hydroxyethylammonium (DORIE) or analogs thereof as disclosed in International Application Publication No. WO 93/03709, which is herein incorporated by reference in its entirety; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 5,283,185, which is herein incorporated by reference in its entirety; cholesteryl-3β-carboxyl-amido-ethylenetrimethylammonium iodide; 1-dimethylamino-3-trimethylammonio-DL-2-propyl-cholesteryl carboxylate iodide; cholesteryl-3-β-carboxyamidoethyleneamine; cholesteryl-3-β-oxysuccinamido-ethylenetrimethylammonium iodide; 1-dimethylamino-3-trimethylammonio-DL-2-propyl-cholesteryl-3-β-oxysuccinate iodide; 2-(2-trimethylammonio)-ethylmethylamino ethyl-cholesteryl-3-β-oxysuccinate iodide; and 3-β-N-(polyethyleneimine)-carbamoylcholesterol.

In some embodiments, the liposomes or lipid bilayers can contain co-lipids that are negatively charged or neutral. As used herein, a “co-lipid” refers to a non-cationic lipid, which includes neutral (uncharged) or anionic lipids. The term “neutral lipid” refers to any of a number of lipid species that exist either in an uncharged or neutral zwitterionic form at physiological pH. The term “anionic lipid” encompasses any of a number of lipid species that carry a net negative charge at physiological pH. Co-lipids can include, but are not limited to, diacylphosphatidylcholine, diacylphosphatidylethanolamine, ceramide, sphingomyelin, cephalin, cholesterol, cerebrosides and diacylglycerols, phospholipid-related materials, such as lecithin, phosphatidylethanolamine, lysolecithin, lysophosphatidylethanolamine, phosphatidylserine, phosphatidylinositol, cardiolipin, phosphatidic acid, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), palmitoyloleyolphosphatidylglycerol (POPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dioleoyl phosphatidic acid (DOPA), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, lysophosphatidylcholine, and dioctadecyldimethyl ammonium bromide and the like. Co-lipids also include polyethylene glycol-based polymers such as PEG 2000, PEG 5000 and polyethylene glycol conjugated to phospholipids or to ceramides, as described in U.S. Pat. No. 5,820,873, herein incorporated by reference in its entirety.

In some embodiments, the liposome of the delivery system complex is a cationic liposome and in other embodiments, the liposome is anionic. The term “cationic liposome” as used herein is intended to encompass any liposome as defined above which has a net positive charge or has a zeta potential of greater than 0 mV at physiological pH. Alternatively, the term “anionic liposome” refers to a liposome as defined above which has a net negative charge or a zeta potential of less than 0 mV at physiological pH. The zeta potential or charge of the liposome can be measured using any method known to one of skill in the art. It should be noted that the liposome itself is the entity that is being determined as cationic or anionic, meaning that the liposome that has a measurable positive charge or negative charge at physiological pH, respectively, can, within an in vivo environment, become attached to other substances or may be associated with other charged components within the aqueous core of the liposome, which can thereby result in the formation of a structure that does not have a net charge. After a delivery system complex comprising a cationic or anionic liposome is produced, molecules such as lipid-PEG conjugates can be post-inserted into the bilayer of the liposome as described elsewhere herein, thus shielding the surface charge of the delivery system complex.

In those embodiments in which the liposome of the delivery system complex is a cationic liposome, the cationic liposome need not be comprised completely of cationic lipids, however, but must be comprised of a sufficient amount of cationic lipids such that the liposome has a positive charge at physiological pH. The cationic liposomes also can contain co-lipids that are negatively charged or neutral, so long as the net charge of the liposome is positive and/or the surface of the liposome is positively charged at physiological pH. In these embodiments, the ratio of cationic lipids to co-lipids is such that the overall charge of the resulting liposome is positive at physiological pH. For example, cationic lipids are present in the cationic liposome at from about 10 mole % to about 100 mole % of total liposomal lipid, in some embodiments, from about 20 mole % to about 80 mole % and, in other embodiments, from about 20 mole % to about 60 mole %. Neutral lipids, when included in the cationic liposome, can be present at a concentration of from about 0 mole % to about 90 mole % of the total liposomal lipid, in some embodiments from about 20 mole % to about 80 mole %, and in other embodiments, from about 40 mole % to about 80 mole %. Anionic lipids, when included in the cationic liposome, can be present at a concentration ranging from about 0 mole % to about 49 mole % of the total liposomal lipid, and in certain embodiments, from about 0 mole % to about 40 mole %.

In some embodiments, the cationic liposome of the delivery system complex comprises a cationic lipid and the neutral co-lipid cholesterol at a 1:1 molar ratio. In some of these embodiments, the cationic lipid comprises DOTAP.

Likewise, in those embodiments in which the liposome of the delivery system complex is an anionic liposome, the anionic liposome need not be comprised completely of anionic lipids, however, but must be comprised of a sufficient amount of anionic lipids such that the liposome has a negative charge at physiological pH. The anionic liposomes also can contain neutral co-lipids or cationic lipids, so long as the net charge of the liposome is negative and/or the surface of the liposome is negatively charged at physiological pH. In these embodiments, the ratio of anionic lipids to neutral co-lipids or cationic lipids is such that the overall charge of the resulting liposome is negative at physiological pH. For example, the anionic lipid is present in the anionic liposome at from about 10 mole % to about 100 mole % of total liposomal lipid, in some embodiments, from about 20 mole % to about 80 mole % and, in other embodiments, from about 20 mole % to about 60 mole %. The neutral lipid, when included in the anionic liposome, can be present at a concentration of from about 0 mole % to about 90 mole % of the total liposomal lipid, in some embodiments from about 20 mole % to about 80 mole %, and in other embodiments, from about 40 mole % to about 80 mole %. The positively charged lipid, when included in the anionic liposome, can be present at a concentration ranging from about 0 mole % to about 49 mole % of the total liposomal lipid, and in certain embodiments, from about 0 mole % to about 40 mole %.

In some embodiments in which the lipid vehicle is a cationic liposome or an anionic liposome, the delivery system complex as a whole has a net positive charge. By “net positive charge” is meant that the positive charges of the components of the delivery system complex (e.g., cationic lipid of liposome, cation of precipitate, cationic bioactive compound) exceed the negative charges of the components of the delivery system complex (e.g., anionic lipid of liposome, anion of precipitate, anionic bioactive compound). It is to be understood, however, that the present invention also encompasses delivery system complexes having a positively charged surface irrespective of whether the net charge of the complex is positive, neutral or even negative. The charge of the surface of a delivery system complex can be measured by the migration of the complex in an electric field by methods known to those in the art, such as by measuring zeta potential (Martin, Swarick, and Cammarata (1983) Physical Pharmacy & Physical Chemical Principles in the Pharmaceutical Sciences, 3rd ed. Lea and Febiger) or by the binding affinity of the delivery system complex to cell surfaces. Complexes exhibiting a positively charged surface have a greater binding affinity to cell surfaces than complexes having a neutral or negatively charged surface. Further, it is to be understood that the positively charged surface can be sterically shielded by the addition of non-ionic polar compounds, for example, polyethylene glycol, as described elsewhere herein.

In particular non-limiting embodiments, the delivery system complex has a charge ratio of positive to negative charge (+:−) of between about 0.5:1 and about 100:1, including but not limited to about 0.5:1, about 1:1, about 2:1, about 3:1, about 4:1, about 5:1, about 6:1, about 7:1, about 8:1, about 9:1, about 10:1, about 15:1, about 20:1, about 40: 1, or about 100:1. In a specific non-limiting embodiment, the +: −charge ratio is about 1:1.

The presently disclosed delivery system complexes comprise liposomes that encapsulate, or coat at least a portion of, a nano-precipitated bioactive compound.

While not being bound by any particular theory or mechanism of action, it is believed the presently disclosed delivery system complexes enter cells through endocytosis and are found in endosomes, which exhibit a relatively low pH (e.g., pH 5.0). Thus, in some embodiments, the bioactive compound is released at endosomal pH. In certain embodiments, the pH level is less than about 6.5, less than about 6.0, less than about 5.5, less than about 5.0, less than about 4.5, or less than about 4.0, including but not limited to, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, or less.

The delivery system complexes can be of any size, so long as the complex is capable of delivering the incorporated bioactive compound to a cell (e.g., in vitro, in vivo), physiological site, or tissue. In some embodiments, the delivery system complex comprises a nanoparticle, wherein the nanoparticle comprises the liposome encapsulating the nano-precipitated bioactive compound. As used herein, the term “nanoparticle” refers to particles of any shape having at least one dimension that is less than about 1000 nm.

In some embodiments, nanoparticles have at least one dimension in the range of about 1 nm to about 1000 nm, including any integer value between 1 nm and 1000 nm (including about 1, 2, 5, 10, 20, 30, 40, 50, 60, 70, 80, 90, 100, 200, 500, and 1000). In certain embodiments, the nanoparticles have at least one dimension that is about 150 nm. Particle size can be determined using any method known in the art, including, but not limited to, sedimentation field flow fractionation, photon correlation spectroscopy, disk centrifugation, and dynamic light scattering (using, for example, a submicron particle sizer such as the NICOMP particle sizing system from AutodilutePAT Model 370; Santa Barbara, Calif.).

As described elsewhere herein, the size of the delivery system complex can be regulated based on the ratio of non-ionic surfactant to organic solvent used during the generation of the water-in-oil microemulsion that comprises the nano-precipitated bioactive compound. Further, the size of the delivery system complexes is dependent upon the ratio of the lipids in the liposome to the nano-precipitate.

Methods for preparing liposomes are known in the art. For example, a review of methodologies of liposome preparation may be found in Liposome Technology (CFC Press NY 1984); Liposomes by Ostro (Marcel Dekker, 1987); Lichtenberg and Barenholz (1988) Methods Biochem Anal. 33:337-462 and U.S. Pat. No. 5,283,185, each of which is herein incorporated by reference in its entirety. For example, cationic lipids and optionally co-lipids can be emulsified by the use of a homogenizer, lyophilized, and melted to obtain multilamellar liposomes. Alternatively, unilamellar liposomes can be produced by the reverse phase evaporation method (Szoka and Papahadjopoulos (1978) Proc. Natl. Acad. Sci. USA 75:4194-4198, which is herein incorporated by reference in its entirety). In some embodiments, the liposomes are produced using thin film hydration (Bangham et al. (1965) J. Mol. Biol. 13:238-252, which is herein incorporated by reference in its entirety). In certain embodiments, the liposome formulation can be briefly sonicated and incubated at 50° C. for a short period of time (e.g., about 10 minutes) prior to sizing (see Templeton et al. (1997) Nature Biotechnology 15:647-652, which is herein incorporated by reference in its entirety).

In some embodiments, the prepared liposome can be sized wherein the liposomes are selected from a population of liposomes based on the size (e.g., diameter) of the liposomes. The liposomes can be sized using techniques such as ultrasonication, high-speed homogenization, and pressure filtration (Hope et al. (1985) Biochimica et Biophysica Acta 812:55; U.S. Pat. Nos. 4,529,561 and 4,737,323, each of which is herein incorporated by reference in its entirety). Sonicating a liposome either by bath or probe sonication produces a progressive size reduction down to small vesicles less than about 0.05 microns in size. Vesicles can be recirculated through a standard emulsion homogenizer to the desired size, typically between about 0.1 microns and about 0.5 microns. The size of the liposomes can be determined by quasi-elastic light scattering (QELS) (Bloomfield (1981) Ann. Rev. Biophys. Bioeng. 10:421-450). The average diameter can be reduced by sonication of the liposomes. Intermittent sonication cycles can be alternated with QELS assessment to guide efficient liposome synthesis. Alternatively, liposomes can be extruded through a small-pore polycarbonate membrane or an asymmetric ceramic membrane to yield a well-defined size distribution. Typically, a suspension is cycled through the membrane one or more times until the desired size distribution is achieved. The complexes can be extruded through successively smaller-pore membranes, to achieve a gradual reduction in size. In particular embodiments, the liposomes are extruded through a membrane having a pore size of about 100 nm.

An emulsion is a dispersion of one liquid in a second immiscible liquid. The term “immiscible” when referring to two liquids refers to the inability of these liquids to be mixed or blended into a homogeneous solution. Two immiscible liquids when added together will always form two separate phases. The organic solvent used in the presently disclosed methods is essentially immiscible with water. Emulsions are essentially swollen micelles, although not all micellar solutions can be swollen to form an emulsion. Micelles are colloidal aggregates of amphipathic molecules that are formed at a well-defined concentration known as the critical micelle concentration. Micelles are oriented with the hydrophobic portions of the lipid molecules at the interior of the micelle and the hydrophilic portions at the exterior surface, exposed to water. The typical number of aggregated molecules in a micelle (aggregation number) has a range from about 50 to about 100. The term “micelles” also refers to inverse or reverse micelles, which are formed in an organic solvent, wherein the hydrophobic portions are at the exterior surface, exposed to the organic solvent and the hydrophilic portion is oriented towards the interior of the micelle.

An oil-in-water (O/W) emulsion consists of droplets of an organic compound (e.g., oil) dispersed in water and a water-in-oil (W/O) emulsion is one in which the phases are reversed and is comprised of droplets of water dispersed in an organic compound (e.g., oil). A water-in-oil emulsion is also referred to herein as a reverse emulsion. Thermodynamically stable emulsions are those that comprise a surfactant (e.g, an amphipathic molecule) and are formed spontaneously. The term “emulsion” can refer to microemulsions or macroemulsions, depending on the size of the particles. Droplet diameters in microemulsions typically range from about 10 to about 100 nm. In contrast, the term macroemulsions refers to droplets having diameters greater than about 100 nm.

It will be evident to one of skill in the art that sufficient amounts of the aqueous solutions, organic solvent, and surfactants are added to the reaction solution to form the water-in-oil emulsion.

Surfactants are added to the reaction solution in order to facilitate the development of and stabilize the water-in-oil microemulsion. Surfactants are molecules that can reduce the surface tension of a liquid. Surfactants have both hydrophilic and hydrophobic properties, and thus, can be solubilized to some extent in either water or organic solvents. Surfactants are classified into four primary groups: cationic, anionic, non-ionic, and zwitterionic. Preferably, the surfactants are non-ionic surfactants. Non-ionic surfactants are those surfactants that have no charge when dissolved or dispersed in aqueous solutions. Thus, the hydrophilic moieties of non-ionic surfactants are uncharged, polar groups. Representative non-limiting examples of non-ionic surfactants suitable for use for the presently disclosed methods and compositions include polyethylene glycol, polysorbates, including but not limited to, polyethoxylated sorbitan fatty acid esters (e.g., Tween® compounds) and sorbitan derivatives (e,g., Span® compounds); ethylene oxide/propylene oxide copolymers (e.g., Pluronic® compounds, which are also known as poloxamers); polyoxyethylene ether compounds, such as those of the Brij® family, including but not limited to polyoxyethylene stearyl ether (also known as polyoxyethylene (100) stearyl ether and by the trade name Brij® 700); ethers of fatty alcohols. In particular embodiments, the non-ionic surfactant comprises octyl phenol ethoxylate (i.e., Triton X-100), which is commercially available from multiple suppliers (e.g., Sigma-Aldrich, St. Louis, Mo.).

Polyethoxylated sorbitan fatty acid esters (polysorbates) are commercially available from multiple suppliers (e.g., Sigma-Aldrich, St Louis, Mo.) under the trade name Tween®, and include, but are not limited to, polyoxyethylene (POE) sorbitan monooleate (Tween® 80), POE sorbitan monostearate (Tween® 60), POE sorbitan monolaurate (Tween® 20), and POE sorbitan monopalmitate (Tween® 40).

Ethylene oxide/propylene oxide copolymers include the block copolymers known as poloxamers, which are also known by the trade name Pluronic® and can be purchased from BASF Corporation (Florham Park, N.J.). Poloxamers are composed of a central hydrophobic chain of polyoxypropylene (poly(propylene oxide)) flanked by two hydrophilic chains of polyoxyethylene (poly(ethylene oxide)) and are represented by the following chemical structure: HO(C₂H₄O)ₐ(C₃H₆O)_{b}(C₂H₄O)ₐH; wherein the C2H4O subunits are ethylene oxide monomers and the C3H6O subunits are propylene oxide monomers, and wherein a and b can be any integer ranging from 20 to 150.

Organic solvents that can be used in the presently disclosed methods include those that are immiscible or essentially immiscible with water. Non-limiting examples of organic solvents that can be used in the presently disclosed methods include chloroform, methanol, ether, ethyl acetate, hexanol, cyclohexane, and dichloromethane. In particular embodiments, the organic solvent is nonpolar or essentially nonpolar.

In some embodiments, mixtures of more than one organic solvent can be used in the presently disclosed methods. In some of these embodiments, the organic solvent comprises a mixture of cyclohexane and hexanol. In particular embodiments, the organic solvent comprises cyclohexane and hexanol at a volume/volume ratio of about 7.5:1.7. As noted elsewhere herein, the non-ionic surfactant can be added to the reaction solution (comprising aqueous solutions of cation, anion, bioactive compound, and organic solvent) separately, or it can first be mixed with the organic solvent and the organic solvent/surfactant mixture can be added to the aqueous solutions of the anion, cation, and bioactive compound. In some of these embodiments, a mixture of cyclohexane, hexanol, and Triton X-100 is added to the reaction solution. In particular embodiments, the volume/volume/volume ratio of the cyclohexane:hexanol:Triton X-100 of the mixture that is added to the reaction solution is about 7.5:1.7:1.8.

It should be noted that the volume/volume ratio of the nonionic surfactant to the organic solvent regulates the size of the water-in-oil microemulsion and therefore, the nano-precipitate contained therein and the resultant delivery system complex, with a greater surfactant:organic solvent ratio resulting in delivery system complexes with larger diameters and smaller surfactant:organic solvent ratios resulting in delivery system complexes with smaller diameters.

The reaction solution may be mixed to form the water-in-oil microemulsion and the solution may also be incubated for a period of time. This incubation step can be performed at room temperature. In some embodiments, the reaction solution is mixed at room temperature for a period of time of between about 5 minutes and about 60 minutes, including but not limited to about 5 minutes, about 10 minutes, about 15 minutes, about 20 minutes, about 25 minutes, about 30 minutes, about 35 minutes, about 40 minutes, about 45 minutes, about 50 minutes, about 55 minutes, and about 60 minutes. In particular embodiments, the reaction solution is mixed at room temperature for about 15 minutes.

In order to complex the nano-precipitated bioactive compound with a liposome, the surface of the nano-precipitate can be charged, either positively or negatively. In some embodiments, the precipitate will have a charged surface following its formation. Those nano-precipitates with positively charged surfaces can be mixed with anionic liposomes, whereas those nano-precipitates with negatively charged surfaces can be mixed with cationic liposomes.

In certain embodiments, the surface charge of the nano-precipitate can be enhanced or reversed using any method known in the art. For example, a nano-precipitate having a positively charged surface can be modified to create a negatively charged surface. Alternatively, a nano-precipitate having a negatively charged surface can be modified to create a positively charged surface.

In those embodiments wherein a nano-precipitate is created having a positive surface charge, the surface charge can be made negative through the addition of sodium citrate to the water-in-oil microemulsion. In some embodiments, sodium citrate is added at a concentration of about 15 mM to the microemulsion. In some of these embodiments, the total volume of the 15 mM sodium citrate added to the microemulsion is about 125 pl. Sodium citrate is especially useful for imparting a negative surface charge to the nano-precipitates because it is non-toxic.

In some embodiments, the precipitate has or is modified to have a zeta potential of less than −10 mV and in certain embodiments, the zeta potential is between about −14 mV and about −20 mV, including but not limited to about −14 mV, about −15 mV, about −16 mV, about −17 mV, about −18 mV, about −19 mV, and about −20 mV. In particular embodiments, the zeta potential of the nano-precipitate is about −16 mV.

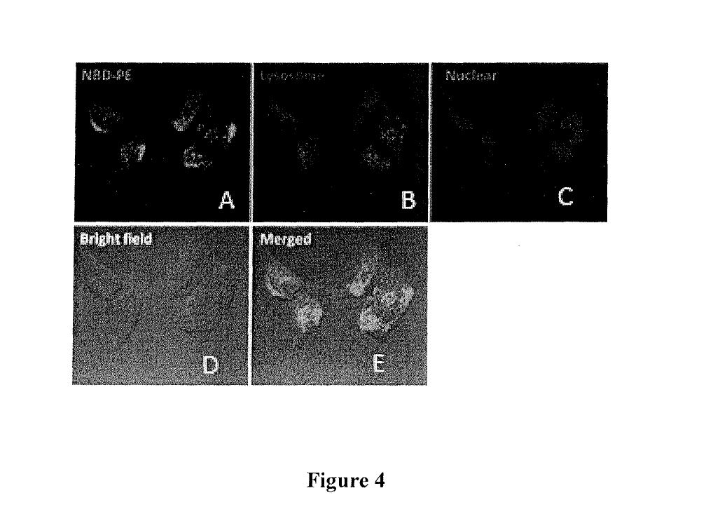

In those embodiments wherein the nano-precipitate has a negatively charged surface, a cationic liposome is complexed with the nano-precipitate. The ratio of the cationic liposome to the nano-precipitate, and/or the bioactive compound can regulate the size and charge of the resultant delivery system complex (see FIG. 4). In those embodiments wherein the bioactive compound comprises a polynucleotide and the zeta potential of the nano-precipitate is about −16 mV, and wherein the liposome comprises a 1:1 molar ratio of DOTAP:cholesterol, a molar ratio of total lipids/polynucleotide of about 973 is used to produce delivery system complexes having a zeta potential of about +40 mV and an average diameter of about 150 nm. In preferred embodiments, the zeta potential of a nanoparticle comprising a liposome is different than the zeta potential of a pure liposome containing the pure lipid, whether the zeta potential is a positive or negative value.

Preferably, the liposomes comprise an outer leaflet comprised of different lipids rather than a single, relatively pure lipid. This also referred to herein as an asymmetric lipid membrane. The asymmetric lipid membrane can shield the charges that would be present on a pure liposome. Preferably, a positive zeta potential is of a lower value than the pure liposome. Preferred zeta potentials of nanoparticles are from about +1 mV to about +40 mV. More preferably, the zeta potential is from about +5 mV to about +25 mV.

Following the production of the emulsion, nano-precipitated bioactive having a lipid coating is purified from the non-ionic surfactant and organic solvent. The nano-precipitate can be purified using any method known in the art, including but not limited to gel filtration chromatography. A nano-precipitate that has been purified from the non-ionic surfactants and organic solvent is a nano-precipitate that is essentially free of non-ionic surfactants or organic solvents (e.g, the nano-precipitate comprises less than 10%, less than 1%, less than 0.1% by weight of the non-ionic surfactant or organic solvent). In some of those embodiments wherein gel filtration is used to purify the nano-precipitate, the precipitate is adsorbed to a silica gel or to a similar type of a stationary phase, the silica gel or similar stationary phase is washed with a polar organic solvent (e.g., ethanol, methanol, acetone, DMSO, DMF) to remove the non-ionic surfactant and organic solvent, and the nano-precipitate is eluted from the silica gel or other solid surface with an aqueous solution comprising a polar organic solvent.

In some of these embodiments, the silica gel is washed with ethanol and the nano-precipitate is eluted with a mixture of water and ethanol. In particular embodiments, the nano-precipitate is eluted with a mixture of water and ethanol, wherein the mixture comprises a volume/volume ratio of between about 1:9 and about 1:1, including but not limited to, about 1:9, about 1:8, about 1:7, about 1:6, about 1:5, about 1:4, about 1:3, about 1:2, and about 1:1. In particular embodiments, the volume/volume ratio of water to ethanol is about 1:3. In some of these embodiments, a mixture comprising 25 ml water and 75 ml ethanol is used for the elution step. Following removal of the ethanol using, for example, rotary evaporation, the nano-precipitate can be dispersed in an aqueous solution (e.g., water) prior to mixing with the prepared liposomes.

In certain embodiments, the methods of making the delivery system complexes can further comprise an additional purification step following the production of the delivery system complexes, wherein the delivery system complexes are purified from excess free liposomes and unencapsulated nano-precipitates. Purification can be accomplished through any method known in the art, including, but not limited to, centrifugation through a sucrose density gradient or other media which is suitable to form a density gradient. It is understood, however, that other methods of purification such as chromatography, filtration, phase partition, precipitation or absorption can also be utilized. In one method, purification via centrifugation through a sucrose density gradient is utilized. The sucrose gradient can range from about 0% sucrose to about 60% sucrose or from about 5% sucrose to about 30% sucrose. The buffer in which the sucrose gradient is made can be any aqueous buffer suitable for storage of the fraction containing the complexes and in some embodiments, a buffer suitable for administration of the complex to cells and tissues.

In some embodiments, a targeted delivery system or a PEGylated delivery system is made as described elsewhere herein, wherein the methods further comprise a post-insertion step following the preparation of the liposome or following the production of the delivery system complex, wherein a lipid-targeting ligand conjugate or a PEGylated lipid is post-inserted into the liposome. Liposomes or delivery system complexes comprising a lipid-targeting ligand conjugate or a lipid-PEG conjugate can be prepared following techniques known in the art, including but not limited to those presented herein (see Experimental section; Ishida et al. (1999) FEBS Lett. 460:129-133; Perouzel et al. (2003) Bioconjug. Chem. 14:884-898, which is herein incorporated by reference in its entirety). The post-insertion step can comprise mixing the liposomes or the delivery system complexes with the lipid-targeting ligand conjugate or a lipid-PEG conjugate and incubating the particles at about 50° C. to about 60° C. for a brief period of time (e.g., about 5 minutes, about 10 minutes). In some embodiments, the delivery system complexes or liposomes are incubated with a lipid-PEG conjugate or a lipid-PEG-targeting ligand conjugate at a concentration of about 5 to about 20 mol %, including but not limited to about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, and about 20 mol %, to form a stealth delivery system. In some of these embodiments, the concentration of the lipid-PEG conjugate is about 10 mol %. The polyethylene glycol moiety of the lipid-PEG conjugate can have a molecular weight ranging from about 100 to about 20,000 g/mol, including but not limited to about 100 g/mol, about 200 g/mol, about 300 g/mol, about 400 g/mol, about 500 g/mol, about 600 g/mol, about 700 g/mol, about 800 g/mol, about 900 g/mol, about 1000 g/mol, about 5000 g/mol, about 10,000 g/mol, about 15,000 g/mol, and about 20,000 g/mol. In certain embodiments, the lipid-PEG conjugate comprises a PEG molecule having a molecular weight of about 2000 g/mol. In some embodiments, the lipid-PEG conjugate comprises DSPE-PEG₂₀₀₀. Lipid-PEG-targeting ligand conjugates can also be post-inserted into liposomes or delivery system complexes using the above described post-insertion methods.

The delivery system complex comprising a nano-precipitated bioactive compound surrounded by a lipid bilayer comprising an inner and an outer leaflet can have a diameter of less than about 100 nm, including but not limited to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, and 100 nm. In particular embodiments, the delivery system complex has a diameter of about 25 to about 30 nm. In particular embodiments, the delivery system complex has a zeta potential of about −17 mV.

The lipid bilayer surrounding the nano-precipitated bioactive compound has an inner and an outer leaflet. In some embodiments, the inner leaflet comprises an amphiphilic lipid having a free phosphate group. Preferably, the amphiphilic lipid having a free phosphate group is dioleoyl phosphatidic acid (DOPA).

The outer leaflet of the lipid bilayer can comprise any type of lipid, but in some embodiments, it comprises a cationic lipid. In particular embodiments, the cationic lipid is DOTAP.

A method of preparing a bioactive compound nano-precipitate encapsulated by a liposome, comprising:

a. contacting a first reverse emulsion comprising a bioactive compound or a precursor thereof with a second reverse emulsion comprising a reagent that is capable of forming a species that can combine with said compound or precursor to form a nano-precipitated bioactive compound, wherein at least one of said first and second reverse emulsion further comprises a neutral or anionic lipid and;

b. allowing said nano-precipitate to form, wherein said nano-precipitate has at least a portion of its surface coated with said neutral or anionic lipid; and

c. contacting said nano-precipitate from (b) with one or more lipids to prepare a bioactive compound nano-precipitate encapsulated by a liposome.

In another embodiment, step (a) can be modified to use a cationic lipid instead of an anionic lipid. The method would then include contacting a first reverse emulsion comprising a bioactive compound or a precursor thereof with a second reverse emulsion comprising a reagent that is capable of forming a species that can combine with said compound or precursor to form a nano-precipitated bioactive compound, wherein at least one of said first and second reverse emulsion further comprises a neutral or cationic lipid. Steps (b) and (c) remain the same except that the nano-precipitate comprises a neutral or cationic lipid. If the nano-precipitate is coated with a cationic lipid, then the second lipid(s) that form the bilayer would appropriately be selected from neutral or anionic lipids.

Useful neutral, anionic and cationic lipids include those listed elsewhere herein. Preferably, the neutral or anionic lipid is DOPA. Useful one or more lipids include co-lipids and cationic lipids listed elsewhere herein. Preferably, the one or more lipids are selected from the group consisting of DOTAP, cholesterol, DSPE-PEG2000 and DSPE-PEG2000-AA.

Useful precursors are bioactive compounds that can be combined with an ion species to form a nano-precipitate in salt form. Such useful bioactive compounds are listed elsewhere herein. Precursors can combine with a cation, such as In⁺³, Gd⁺³, Mg⁺², Zn⁺² and Ba⁺² or an anion, such as a halide, to form a nano-precipitate in situ, i.e., during mixing of the reverse micro-emulsions. In the latter instance, preferably, the precursor is cis-diaminedihydroplatinum(II).

The method can include purifying and washing steps as disclosed herein. These steps employ solvents, washes and purification procedures described herein. In particular, the method further comprises a washing and/or purifying step after (b) and before (c). Generally, the methods can comprise mixing a first reverse microemulsion and a second reverse microemulsion to form a salt of a bioactive compound that itself is a nano-precipitate having a lipid coating, this nano-precipitate will have an outer leaflet lipid layer added in subsequent steps to form a nano-precipitate having a lipid bi-layer coat; washing the nano-precipitate; mixing the nano-precipitate in a volatile, organic solvent to form a nano-precipitate/solvent mixture; adding a lipid to the nano-precipitate/solvent mixture; and evaporating the volatile, organic solvent to produce said delivery system complex. Scheme 1 depicts a synthetic route for preparing exemplary delivery complexes.

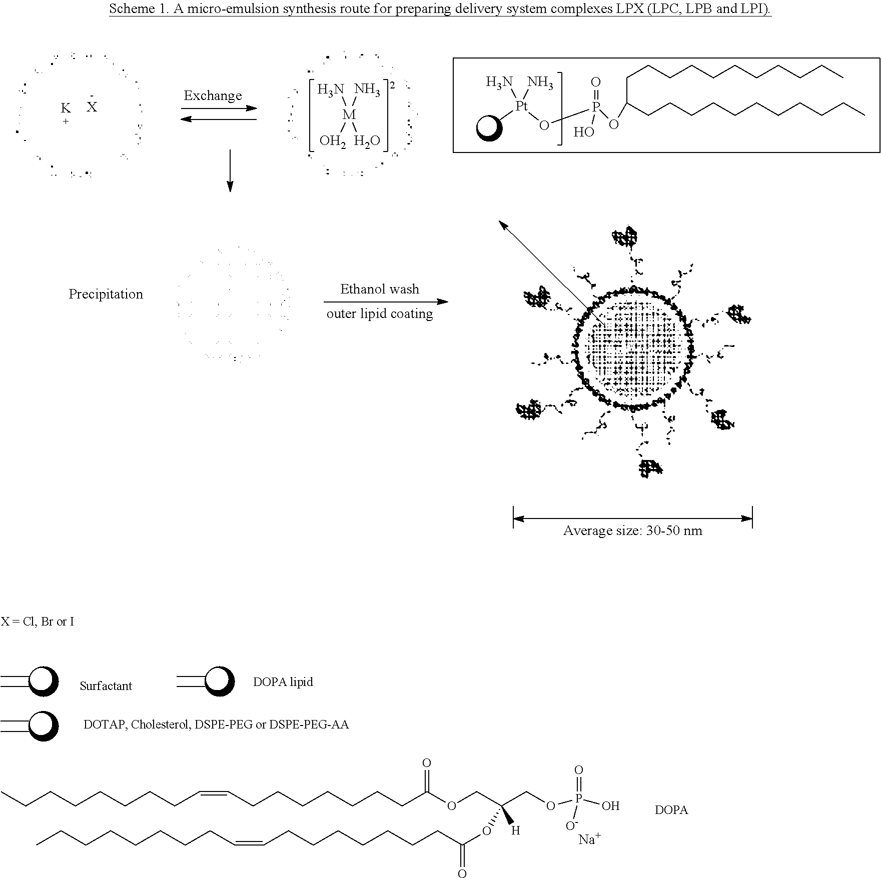

In some embodiments, the first reverse microemulsion has the same or different pH as the second reverse microemulsion.

The method can further comprise producing the first reverse microemulsion, which can include providing a solution comprising a bioactive compound or a precursor thereof, and mixing the solution with a non-ionic surfactant and an organic solvent. In particular, the first microemulsion can contain triton X-100, IGEPAL 520, which are both well-known in the art, and hexanol as co-surfactants in an organic solvent.

In some embodiments, the organic solvent is hexanol and/or cyclohexane. In particular embodiments, the organic solvent comprises cyclohexane and hexanol at a volume-to-volume ratio of about 78:11.

The non-ionic surfactant can be any non-ionic surfactant, including those non-limiting examples provided elsewhere herein, but in certain embodiments, the non-ionic surfactant is Triton-X 100. In particular embodiments, the aqueous solution comprising calcium chloride is mixed with a solution of cyclohexane, hexanol, and Triton-X 100 at a volume/volume/volume ratio of about 78:11:11.

The method can further comprise providing a second reverse emulsion that contains the species that will combine with the bioactive compound or precursor of a bioactive compound to form a nano-precipitated bioactive compound. The species can be a cation or anion. In embodiments, the cation is a monovalent, divalent or a trivalent cation. The cations that used to form the salt nano-precipitates can be radioactive isotopes which will allow imaging of the lesion. An example is ¹¹¹In which can be imaged by SPECT. Gd⁺³ can also be used as an MRI agent. Thus, the resulting liposomes will carry both a therapeutic and an imaging agent for theranostic nanomedicines. In embodiments, the anion is a monovalent, divalent or a trivalent anion. In particular embodiments, the anion is a halide anion (fluoride (F⁻), chloride (Cl⁻), bromide (Br⁻) and iodide (I⁻)).

The second reverse microemulsion will comprise the ion species (by way of adding its precursor such as a halide salt) and a neutral and/or anionic lipid. Preferably, the lipid is DOPA. The second reverse emulsion will be an emulsion that can further comprise a non-ionic surfactant, and an organic solvent.

Again, the organic solvent can comprise hexanol and/or cyclohexane. In particular embodiments, the organic solvent comprises cyclohexane and hexanol at a volume-to-volume ratio of about 78:11.

Likewise, the non-ionic surfactant used to produce the second reverse microemulsion can be any non-ionic surfactant, including those non-limiting examples provided elsewhere herein, but in certain embodiments, the non-ionic surfactant is Triton-X 100. In particular embodiments, the aqueous solution comprising sodium phosphate and the anionic lipid is mixed with a solution of cyclohexane, hexanol, and Triton-X 100 at a volume/volume/volume ratio of about 78:11:11.

The volatile, organic solvent within which the nano-precipitate is mixed can be ethanol or chloroform. In some embodiments, the nano-precipitate is washed with ethanol, and the washing step can be performed about 1-5 times, including 1, 2, 3, 4, and 5.

The monolayer lipid nano-precipitate can be encapsulated with an outer leaflet comprising one or more of cholesterol, a cationic lipid such as DOTAP or a neutral lipid, such as dioleoyl phosphatidylcholine by combining one or more to the mixture containing the monolayer lipid nano-precipitate. In some embodiments, the outer leaflet comprises a lipid-polyethylene glycol (lipid-PEG) conjugate, a lipid-targeting ligand conjugate, or a combination thereof. In certain embodiments, a mixture of neutral lipids (e.g., DOPC) and a lipid-PEG conjugate, a lipid-targeting ligand conjugate, or a combination thereof is at a molar ratio of 10 neutral lipid (e.g., DOPC) to 1 lipid-PEG conjugate, lipid targeting ligand conjugate, or combination thereof (e.g., DSPE-PEG-AA). Alternatively, the lipid-PEG conjugate, lipid targeting ligand conjugate, or a combination thereof can be added to the outer leaflet of the lipid bilayer through post-insertion described elsewhere herein.

II. Low Solubility Bioactive Compounds

By “low solubility bioactive compound” is intended any agent that has a desired effect (e.g., therapeutic effect) on a living cell, tissue, or organism, or an agent that can desirably interact with a component (e.g., enzyme) of a living cell, tissue, or organism and that is not appreciably soluble in water and oil or a bioactive compound that can be soluble in water and/or oil, such as a precursor, that is capable of combining with an ion to form a nano-precipitate that is not appreciably solubilized in water and oil. The low solubility bioactive agents are also not appreciably solubilized under physiological conditions. Preferred bioactive agents can be formed into nano-precipitates and have a solubility of less than 10 mg/ml in water at 25° C. Unlike existing technologies, the subject matter described herein advantageously utilizes low-soluble or insoluble active agents and nano-precipitates thereof. Accordingly, it is preferred that the bioactive compound or its nano-precipitate has a solubility of less than 8 mg/ml in water at 25° C. More preferably, the bioactive compound or its nano-precipitate has a solubility of less than 5 mg/ml in water at 25° C. Most preferably, the bioactive compound or its nano-precipitate has a solubility of less than 3 mg/ml in water at 25° C.

In embodiments, low solubility bioactive compounds include compounds that are essentially insoluble in water and oil. The bioactive compounds useful in the delivery complexes described herein combine with an ion (ionic species), e.g. an anion, such as a halide, or a cation, to form a nano-precipitate. In embodiments, the nano-precipitate consists essentially of the bioactive compound and the lipid. In other words, there is no other ionic core material present that is a seeding material.

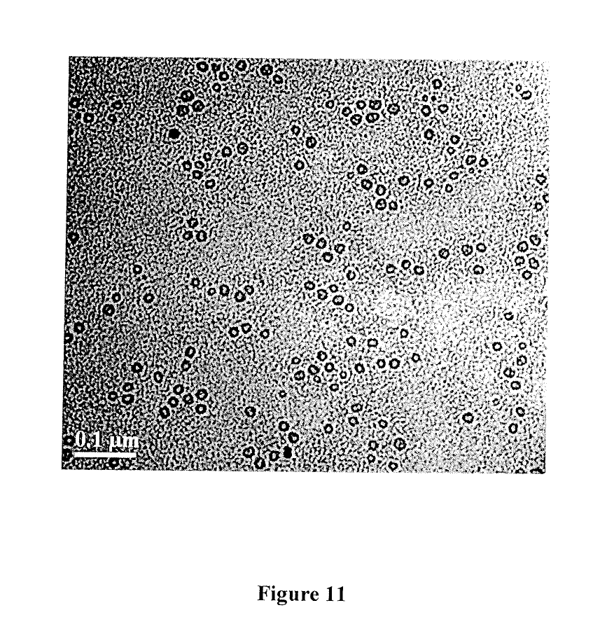

It is noted that soluble bioactive compounds and, in particular, soluble precursor compounds can be utilized when they are prepared according to the methods described herein to form nano-precipitates as described herein. An example is the precursor of cisplatin that is combined with a halide salt to from a nano-precipitate. Another example is etoposide phosphate (Etopophos®), which is water soluble. However, using the methods described herein, etoposide phosphate contained in a first reverse emulsion can be contacted with InC1₃ contained in a second reverse emulsions. The In salt of etoposide phosphate formed therein is insoluble and formed a nano-precipitate (FIG. 11).

Bioactive compounds can include, but are not limited to, polynucleotides, polypeptides, polysaccharides, organic and inorganic small molecules. The term “bioactive compound” encompasses both naturally occurring and synthetic bioactive compounds. The term “bioactive compound” can refer to a detection or diagnostic agent that interacts with a biological molecule to provide a detectable readout that reflects a particular physiological or pathological event.

Exemplary compounds include inorganic complexes such as platinum coordination complexes that include cisplatin, carboplatin, hydroxyurea, amsacrine, procarbazine, mitotane, mitoxantrone, levamisole, and hexamethylmelamine.

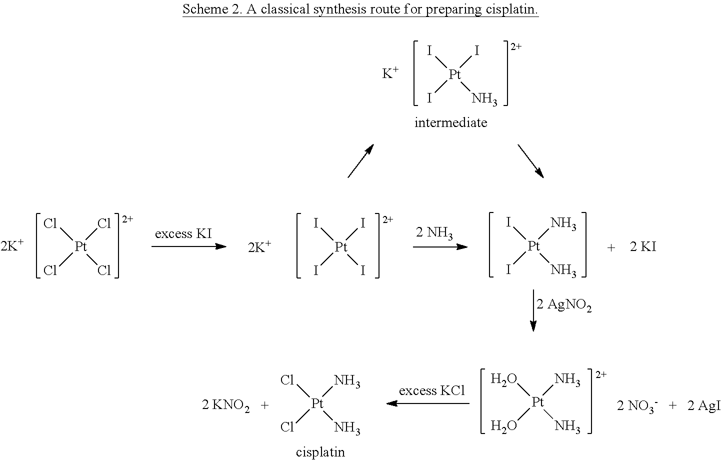

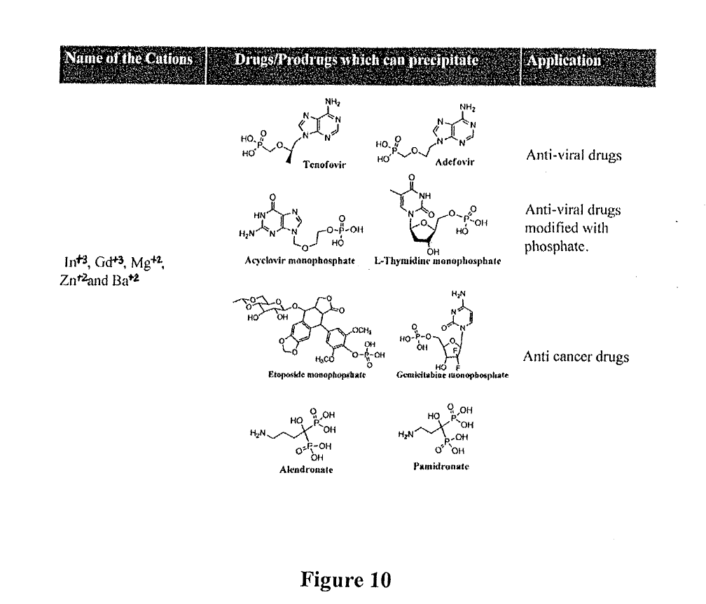

Other specific bioactive compounds and their ion pairs that can form nano-precipitates are shown in FIG. 10. The essentially insoluble bioactive compound can be a chemotherapeutic drug. In other embodiments, the bioactive compound comprises a polynucleotide of interest or a polypeptide of interest, such as a silencing element (e.g., siRNA) as described elsewhere herein.

The bioactive compound of the delivery system can be a drug, including, but not limited to, antimicrobials, antibiotics, antimycobacterials, antifungals, antivirals, neoplastic agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholinergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenics, non-steroidal anti-inflammatory agents, steroidal anti-inflammatory agents, antioxidants, vaso-active agents, bone-active agents, antiarthritics, and diagnostic agents. Preferred antiviral drugs include tenofovir, adefovir, acyclovir monophosphate and L-thymidine monophosphate. In a preferred embodiment, the bioactive compound is an anticancer drug. In this embodiment, it is preferred that the bioactive compound is cisplatin and its analogues, etoposide monophosphate, alendronate, pamidronate, and gemcitabine monophosphate and salts, esters, conformers and produgs thereof.

In those embodiments wherein the bioactive compound comprises a polynucleotide, the delivery system complex can be referred to as a “polynucleotide delivery system” or “polynulceotide delivery system complex.”

As used herein, the term “deliver” refers to the transfer of a substance or molecule (e.g., a polynucleotide) to a physiological site, tissue, or cell. This encompasses delivery to the intracellular portion of a cell or to the extracellular space. Delivery of a polynucleotide into the intracellular portion of a cell is also often referred to as “transfection.”

As used herein, the term “intracellular” or “intracellularly” has its ordinary meaning as understood in the art. In general, the space inside of a cell, which is encircled by a membrane, is defined as “intracellular” space. Similarly, as used herein, the term “extracellular” or “extracellularly” has its ordinary meaning as understood in the art. In general, the space outside of the cell membrane is defined as “extracellular” space.

The term “polynucleotide” is intended to encompass a singular nucleic acid, as well as plural nucleic acids, and refers to a nucleic acid molecule or construct, e.g., messenger RNA (mRNA), plasmid DNA (pDNA), or short interfering RNA (siRNA). A polynucleotide can be single-stranded or double-stranded, linear or circular. A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments or synthetic analogues thereof, present in a polynucleotide. The term “polynucleotide” can refer to an isolated polynucleotide, including recombinant polynucleotides maintained in heterologous host cells or purified (partially or substantially) polynucleotides in solution. Polynucleotides or nucleic acids according to the present invention further include such molecules produced synthetically. Polynucleotides can also include isolated expression vectors, expression constructs, or populations thereof. “Polynucleotide” can also refer to amplified products of itself, as in a polymerase chain reaction. The “polynucleotide” can contain modified nucleic acids, such as phosphorothioate, phosphate, ring atom modified derivatives, and the like. The “polynucleotide” can be a naturally occurring polynucleotide (i.e., one existing in nature without human intervention), or a recombinant polynucleotide (i.e., one existing only with human intervention). While the terms “polynucleotide” and “oligonucleotide” both refer to a polymer of nucleotides, as used herein, an oligonucleotide is typically less than 100 nucleotides in length.

As used herein, the term “polynucleotide of interest” refers to a polynucleotide that is to be delivered to a cell to elicit a desired effect in the cell (e.g., a therapeutic effect, a change in gene expression). A polynucleotide of interest can be of any length and can include, but is not limited to, a polynucleotide comprising a coding sequence for a polypeptide of interest or a polynucleotide comprising a silencing element. In certain embodiments, when the polynucleotide is expressed or introduced into a cell, the polynucleotide of interest or polypeptide encoded thereby has therapeutic activity.

In some embodiments, delivery system complexes comprise a polynucleotide of interest comprising a coding sequence for a polypeptide of interest.

For the purposes of the present invention, a “coding sequence for a polypeptide of interest” or “coding region for a polypeptide of interest” refers to the polynucleotide sequence that encodes that polypeptide. As used herein, the terms “encoding” or “encoded” when used in the context of a specified nucleic acid mean that the nucleic acid comprises the requisite information to direct translation of the nucleotide sequence into a specified polypeptide. The information by which a polypeptide is encoded is specified by the use of codons. The “coding region” or “coding sequence” is the portion of the nucleic acid that consists of codons that can be translated into amino acids. Although a “stop codon” or “translational termination codon” (TAG, TGA, or TAA) is not translated into an amino acid, it can be considered to be part of a coding region Likewise, a transcription initiation codon (ATG) may or may not be considered to be part of a coding region. Any sequences flanking the coding region, however, for example, promoters, ribosome binding sites, transcriptional terminators, introns, and the like, are not considered to be part of the coding region. In some embodiments, however, while not considered part of the coding region per se, these regulatory sequences and any other regulatory sequence, particularly signal sequences or sequences encoding a peptide tag, may be part of the polynucleotide sequence encoding the polypeptide of interest. Thus, a polynucleotide sequence encoding a polypeptide of interest comprises the coding sequence and optionally any sequences flanking the coding region that contribute to expression, secretion, and/or isolation of the polypeptide of interest.

The term “expression” has its meaning as understood in the art and refers to the process of converting genetic information encoded in a gene or a coding sequence into RNA (e.g., mRNA, rRNA, tRNA, or snRNA) through “transcription” of a polynucleotide (e.g., via the enzymatic action of an RNA polymerase), and for polypeptide-encoding polynucleotides, into a polypeptide through “translation” of mRNA. Thus, an “expression product” is, in general, an RNA transcribed from the gene (e.g., either pre- or post-processing) or polynucleotide or a polypeptide encoded by an RNA transcribed from the gene (e.g., either pre- or post-modification).

As used herein, the term “polypeptide” or “protein” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms.

The term “polypeptide of interest” refers to a polypeptide that is to be delivered to a cell or is encoded by a polynucleotide that is to be delivered to a cell to elicit a desired effect in the cell (e.g., a therapeutic effect). The polypeptide of interest can be of any species and of any size. In certain embodiments, however, the protein or polypeptide of interest is a therapeutically useful protein or polypeptide. In some embodiments, the protein can be a mammalian protein, for example a human protein. In certain embodiments, the polynucleotide comprises a coding sequence for a tumor suppressor or a cytotoxin (e.g., diphtheria toxin (DT), Pseudomonas exotoxin A (PE), pertussis toxin (PT), and the pertussis adenylate cyclase (CYA)).

The term “tumor suppressor” refers to a polypeptide or a gene that encodes a polypeptide that is capable of inhibiting the development, growth, or progression of cancer. Tumor suppressor polypeptides include those proteins that regulate cellular proliferation or responses to cellular and genomic damage, or induce apoptosis. Non-limiting examples of tumor suppressor genes include p53, p110Rb, and p72. Thus, in some embodiments, the delivery system complexes of the present invention comprise a polynucleotide of interest comprising a coding sequence for a tumor suppressor.

Extensive sequence information required for molecular genetics and genetic engineering techniques is widely publicly available. Access to complete nucleotide sequences of mammalian, as well as human, genes, cDNA sequences, amino acid sequences and genomes can be obtained from GenBank at the website www.ncbi.nlm.nih.gov/Entrez. Additional information can also be obtained from GeneCards, an electronic encyclopedia integrating information about genes and their products and biomedical applications from the Weizmann Institute of Science Genome and Bioinformatics (bioinformatics.weizmann.ac.il/cards), nucleotide sequence information can be also obtained from the EMBL Nucleotide Sequence Database (www.ebi.ac.uk/embl) or the DNA Databank or Japan (DDBJ, www.ddbi.nig.ac.jp). Additional sites for information on amino acid sequences include Georgetown's protein information resource website (www.pir.georgetown.edu) and Swiss-Prot (au.expasy.org/sprot/sprot-top.html).

In some embodiments, the polynucleotide of interest of the delivery system complexes of the invention comprises a silencing element, wherein expression or introduction of the silencing element into a cell reduces the expression of a target polynucleotide or polypeptide encoded thereby.

The terms “introduction” or “introduce” when referring to a polynucleotide or silencing element refers to the presentation of the polynucleotide or silencing element to a cell in such a manner that the polynucleotide or silencing element gains access to the intracellular region of the cell.

As used herein, the term “silencing element” refers to a polynucleotide, which when expressed or introduced into a cell is capable of reducing or eliminating the level of expression of a target polynucleotide sequence or the polypeptide encoded thereby. The silencing element can comprise or encode an antisense oligonucleotide or an interfering RNA (RNAi). The term “interfering RNA” or “RNAi” refers to any RNA molecule which can enter an RNAi pathway and thereby reduce the expression of a target polynucleotide of interest. The RNAi pathway features the Dicer nuclease enzyme and RNA-induced silencing complexes (RISC) that function to degrade or block the translation of a target mRNA. RNAi is distinct from antisense oligonucleotides that function through “antisense” mechanisms that typically involve inhibition of a target transcript by a single-stranded oligonucleotide through an RNase H-mediated pathway. See, Crooke (ed.) (2001) “Antisense Drug Technology: Principles, Strategies, and Applications” (1st ed), Marcel Dekker; ISBN: 0824705661; 1st edition.

As used herein, a “target polynucleotide” comprises any polynucleotide sequence that one desires to decrease the level of expression. By “reduces” or “reducing” the expression level of a polynucleotide or a polypeptide encoded thereby is intended to mean, the level of the polynucleotide or the encoded polypeptide is statistically lower than the target polynucleotide level or encoded polypeptide level in an appropriate control which is not exposed to the silencing element. In particular embodiments, reducing the target polynucleotide level and/or the encoded polypeptide level according to the presently disclosed subject matter results in less than 95%, less than 90%, less than 80%, less than 70%, less than 60%, less than 50%, less than 40%, less than 30%, less than 20%, less than 10%, or less than 5% of the target polynucleotide level, or the level of the polypeptide encoded thereby in an appropriate control. Methods to assay for the level of the RNA transcript, the level of the encoded polypeptide, or the activity of the polynucleotide or polypeptide are discussed elsewhere herein.

A particular silencing element may specifically reduce the expression of a particular target polynucleotide or a polypeptide encoded thereby or the silencing element may reduce the expression of multiple target polynucleotides or polypeptides encoded thereby.

In some embodiments, the target polynucleotide is an oncogene or a proto-oncogene. The term “oncogene” is used herein in accordance with its art-accepted meaning to refer to those polynucleotide sequences that encode a gene product that contributes to cancer initiation or progression. The term “oncogene” encompasses proto-oncogenes, which are genes that do not contribute to carcinogenesis under normal circumstances, but that have been mutated, overexpressed, or activated in such a manner as to function as an oncogene. Non-limiting examples of oncogenes include growth factors or mitogens (e.g., c-Sis), receptor tyrosine kinases (e.g., epidermal growth factor receptor (EGFR), platelet-derived growth factor receptor (PDGFR), vascular endothelial growth factor receptor (VEGFR), HER2/neu), cytoplasmic tyrosine kinases (e.g., src, Abl), cytoplasmic serine/threonine kinases (e.g., raf kinase, cyclin-dependent kinases), regulatory GTPases (e.g., ras), and transcription factors (e.g., myc). In some embodiments, the target polynucleotide is EGFR.

The term “complementary” is used herein in accordance with its art-accepted meaning to refer to the capacity for precise pairing via hydrogen bonds (e.g., Watson-Crick base pairing or Hoogsteen base pairing) between two nucleosides, nucleotides or nucleic acids, and the like. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position (where position may be defined relative to either end of either nucleic acid, generally with respect to a 5′ end). The nucleotides located opposite one another can be referred to as a “base pair.” A complementary base pair contains two complementary nucleotides, e.g., A and U, A and T, G and C, and the like, whereas a noncomplementary base pair contains two noncomplementary nucleotides (also referred to as a mismatch). Two polynucleotides are said to be complementary to each other when a sufficient number of corresponding positions in each molecule are occupied by nucleotides that hydrogen bond with each other, i.e., a sufficient number of base pairs are complementary.

As used herein, the term “gene” has its meaning as understood in the art. In general, a gene is taken to include gene regulatory sequences (e.g., promoters, enhancers, and the like) and/or intron sequences, in addition to coding sequences (open reading frames). It will further be appreciated that definitions of “gene” include references to nucleic acids that do not encode proteins but rather encode functional RNA molecules, or precursors thereof, such as microRNA or siRNA precursors, tRNAs, and the like.

The term “hybridize” as used herein refers to the interaction between two complementary nucleic acid sequences in which the two sequences remain associated with one another under appropriate conditions.

A silencing element can comprise the interfering RNA or antisense oligonucleotide, a precursor to the interfering RNA or antisense oligonucleotide, a template for the transcription of an interfering RNA or antisense oligonucleotide, or a template for the transcription of a precursor interfering RNA or antisense oligonucleotide, wherein the precursor is processed within the cell to produce an interfering RNA or antisense oligonucleotide. Thus, for example, a dsRNA silencing element includes a dsRNA molecule, a transcript or polyribonucleotide capable of forming a dsRNA, more than one transcript or polyribonucleotide capable of forming a dsRNA, a DNA encoding a dsRNA molecule, or a DNA encoding one strand of a dsRNA molecule. When the silencing element comprises a DNA molecule encoding an interfering RNA, it is recognized that the DNA can be transiently expressed in a cell or stably incorporated into the genome of the cell. Such methods are discussed in further detail elsewhere herein.

The silencing element can reduce or eliminate the expression level of a target polynucleotide or encoded polypeptide by influencing the level of the target RNA transcript, by influencing translation, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional interfering RNA that are capable of reducing or eliminating the level of a sequence of interest are disclosed elsewhere herein.

Any region of the target polynucleotide can be used to design a domain of the silencing element that shares sufficient sequence identity to allow for the silencing element to decrease the level of the target polynucleotide or encoded polypeptide. For instance, the silencing element can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof.

The ability of a silencing element to reduce the level of the target polynucleotide can be assessed directly by measuring the amount of the target transcript using, for example, Northern blots, nuclease protection assays, reverse transcription (RT)-PCR, real-time RT-PCR, microarray analysis, and the like. Alternatively, the ability of the silencing element to reduce the level of the target polynucleotide can be measured directly using a variety of affinity-based approaches (e.g., using a ligand or antibody that specifically binds to the target polypeptide) including, but not limited to, Western blots, immunoassays, ELISA, flow cytometry, protein microarrays, and the like. In still other methods, the ability of the silencing element to reduce the level of the target polynucleotide can be assessed indirectly, e.g., by measuring a functional activity of the polypeptide encoded by the transcript or by measuring a signal produced by the polypeptide encoded by the transcript.

Various types of silencing elements are discussed in further detail below.

In one embodiment, the silencing element comprises or encodes a double stranded RNA molecule. As used herein, a “double stranded RNA” or “dsRNA” refers to a polyribonucleotide structure formed either by a single self-complementary RNA molecule or a polyribonucleotide structure formed by the expression of least two distinct RNA strands. Accordingly, as used herein, the term “dsRNA” is meant to encompass other terms used to describe nucleic acid molecules that are capable of mediating RNA interference or gene silencing, including, for example, small RNA (sRNA), short-interfering RNA (siRNA), double-stranded RNA (dsRNA), micro-RNA (miRNA), hairpin RNA, short hairpin RNA (shRNA), and others. See, for example, Meister and Tuschl (2004) Nature 431:343-349 and Bonetta et al. (2004) Nature Methods 1:79-86.

In specific embodiments, at least one strand of the duplex or double-stranded region of the dsRNA shares sufficient sequence identity or sequence complementarity to the target polynucleotide to allow for the dsRNA to reduce the level of expression of the target polynucleotide or encoded polypeptide. As used herein, the strand that is complementary to the target polynucleotide is the “antisense strand,” and the strand homologous to the target polynucleotide is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. For example, the hairpin RNA molecule that hybridizes with itself to form a hairpin structure can comprise a single-stranded loop region and a base-paired stem. The base-paired stem region can comprise a sense sequence corresponding to all or part of the target polynucleotide and further comprises an antisense sequence that is fully or partially complementary to the sense sequence. Thus, the base-paired stem region of the silencing element can determine the specificity of the silencing. See, for example, Chuang and Meyerowitz (2000) Proc. Natl. Acad. Sci. USA 97:4985-4990, herein incorporated by reference. A transient assay for the efficiency of hpRNA constructs to silence gene expression in vivo has been described by Panstruga et al. (2003) Mol. Biol. Rep. 30:135-140, herein incorporated by reference.

A “short interfering RNA” or “siRNA” comprises an RNA duplex (double-stranded region) and can further comprise one or two single-stranded overhangs, e.g., 3′ or 5′ overhangs. The duplex can be approximately 19 base pairs (bp) long, although lengths between 17 and 29 nucleotides, including 17, 18, 19, 20, 21, 22, 23, 24, 25, 26, 27, 28, and 29 nucleotides, can be used. An siRNA can be formed from two RNA molecules that hybridize together or can alternatively be generated from a single RNA molecule that includes a self-hybridizing portion. The duplex portion of an siRNA can include one or more bulges containing one or more unpaired and/or mismatched nucleotides in one or both strands of the duplex or can contain one or more noncomplementary nucleotide pairs. One strand of an siRNA (referred to herein as the antisense strand) includes a portion that hybridizes with a target transcript. In certain embodiments, one strand of the siRNA (the antisense strand) is precisely complementary with a region of the target transcript over at least about 17 nucleotides, 18 nucleotides, 19 nucleotides, 20 nucleotides, 21 nucleotides, or more meaning that the siRNA antisense strand hybridizes to the target transcript without a single mismatch (i.e., without a single noncomplementary base pair) over that length. In other embodiments, one or more mismatches between the siRNA antisense strand and the targeted portion of the target transcript can exist. In embodiments in which perfect complementarity is not achieved, any mismatches between the siRNA antisense strand and the target transcript can be located at or near the 3′ end of the siRNA antisense strand. For example, in certain embodiments, nucleotides 1-9, 2-9, 2-10, and/or 1-10 of the antisense strand are perfectly complementary to the target.

Considerations for the design of effective siRNA molecules are discussed in McManus et al. (2002) Nature Reviews Genetics 3: 737-747 and in Dykxhoorn et al. (2003) Nature Reviews Molecular Cell Biology 4: 457-467. Such considerations include the base composition of the siRNA, the position of the portion of the target transcript that is complementary to the antisense strand of the siRNA relative to the 5′ and 3′ ends of the transcript, and the like. A variety of computer programs also are available to assist with selection of siRNA sequences, e.g., from Ambion (web site having URL www.ambion.com), at the web site having the URL www.sinc.sunysb.edu/Stu/shilin/rnai.html. Additional design considerations that also can be employed are described in Semizarov et al. Proc. Natl. Acad. Sci. 100: 6347-6352.

The term “short hairpin RNA” or “shRNA” refers to an RNA molecule comprising at least two complementary portions hybridized or capable of hybridizing to form a double-stranded (duplex) structure sufficiently long to mediate RNAi (generally between approximately 17 and 29 nucleotides in length, including 17, 18, 19, 20, 21, 22, 23, 24, 25, 26, 27, 28, and 29 nucleotides in length, and in some embodiments, typically at least 19 base pairs in length), and at least one single-stranded portion, typically between approximately 1 and 20 or 1 to 10 nucleotides in length that forms a loop connecting the two nucleotides that form the base pair at one end of the duplex portion. The duplex portion can, but does not require, one or more bulges consisting of one or more unpaired nucleotides. In specific embodiments, the shRNAs comprise a 3′ overhang. Thus, shRNAs are precursors of siRNAs and are, in general, similarly capable of inhibiting expression of a target transcript.

In particular, RNA molecules having a hairpin (stem-loop) structure can be processed intracellularly by Dicer to yield an siRNA structure referred to as short hairpin RNAs (shRNAs), which contain two complementary regions that hybridize to one another (self-hybridize) to form a double-stranded (duplex) region referred to as a stem, a single-stranded loop connecting the nucleotides that form the base pair at one end of the duplex, and optionally an overhang, e.g., a 3′ overhang. The stem can comprise about 19, 20, or 21 bp long, though shorter and longer stems (e.g., up to about 29 nt) also can be used. The loop can comprise about 1-20, including 1, 2, 3, 4, 5, 6, 7, 8, 9, 10, 11, 12, 13, 14, 15, 16, 17, 18, 19, 20 nt, about 4-10, or about 6-9 nt. The overhang, if present, can comprise approximately 1-20 nt or approximately 2-10 nt. The loop can be located at either the 5′ or 3′ end of the region that is complementary to the target transcript whose inhibition is desired (i.e., the antisense portion of the shRNA).

Although shRNAs contain a single RNA molecule that self-hybridizes, it will be appreciated that the resulting duplex structure can be considered to comprise sense and antisense strands or portions relative to the target mRNA and can thus be considered to be double-stranded. It will therefore be convenient herein to refer to sense and antisense strands, or sense and antisense portions, of an shRNA, where the antisense strand or portion is that segment of the molecule that forms or is capable of forming a duplex with and is complementary to the targeted portion of the target polynucleotide, and the sense strand or portion is that segment of the molecule that forms or is capable of forming a duplex with the antisense strand or portion and is substantially identical in sequence to the targeted portion of the target transcript. In general, considerations for selection of the sequence of the antisense strand of an shRNA molecule are similar to those for selection of the sequence of the antisense strand of an siRNA molecule that targets the same transcript.

In one embodiment, the silencing element comprises or encodes an miRNA or an miRNA precursor. “MicroRNAs” or “miRNAs” are regulatory agents comprising about 19 ribonucleotides which are highly efficient at inhibiting the expression of target polynucleotides. See, for example, Saetrom et al. (2006) Oligonucleotides 16:115-144, Wang et al. (2006) Mol. Cell 22:553-60, Davis et al. (2006) Nucleic Acid Research 34:2294-304, Pasquinelli (2006) Dev. Cell 10:419-24, all of which are herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

It is recognized that various forms of an miRNA can be transcribed including, for example, the primary transcript (termed the “pri-miRNA”) which is processed through various nucleolytic steps to a shorter precursor miRNA (termed the “pre-miRNA”); the pre-miRNA; or the final (mature) miRNA, which is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (McManus et al. (2002) RNA 8:842-50). In specific embodiments, 2-8 nucleotides of the miRNA are perfectly complementary to the target. A large number of endogenous human miRNAs have been identified. For structures of a number of endogenous miRNA precursors from various organisms, see Lagos-Quintana et al. (2003) RNA 9(2):175-9; see also Bartel (2004) Cell 116:281-297.

A miRNA or miRNA precursor can share at least about 80%, 85%, 90%, 91%. 92%, 93%, 94%, 95%, 96%, 97%, 98%, 99%, or 100% sequence complementarity with the target transcript for a stretch of at least about 15, 16, 17, 18, 19, 20, 21, 22, 23, 24, 25, 26, 27, 28, 29, or 30 nucleotides. In specific embodiments, the region of precise sequence complementarity is interrupted by a bulge. See, Ruvkun (2001) Science 294: 797-799, Zeng et al. (2002) Molecular Cell 9:1-20, and Mourelatos et al. (2002) Genes Dev 16:720-728.

In some embodiments, the silencing element comprises or encodes an antisense oligonucleotide. An “antisense oligonucleotide” is a single-stranded nucleic acid sequence that is wholly or partially complementary to a target polynucleotide, and can be DNA, or its RNA counterpart (i.e., wherein T residues of the DNA are U residues in the RNA counterpart).

The antisense oligonucleotides of this invention are designed to be hybridizable with target RNA (e.g., mRNA) or DNA. For example, an oligonucleotide (e.g., DNA oligonucleotide) that hybridizes to a mRNA molecule can be used to target the mRNA for

RnaseH digestion. Alternatively, an oligonucleotide that hybridizes to the translation initiation site of an mRNA molecule can be used to prevent translation of the mRNA. In another approach, oligonucleotides that bind to double-stranded DNA can be administered. Such oligonucleotides can form a triplex construct and inhibit the transcription of the DNA. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.). Such oligonucleotides of the invention can be constructed using the base-pairing rules of triple helix formation and the nucleotide sequences of the target genes.

As non-limiting examples, antisense oligonucleotides can be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; and 3′ coding region. In some embodiments, the complementary oligonucleotide is designed to hybridize to the most unique 5′ sequence of a gene, including any of about 15-35 nucleotides spanning the 5′ coding sequence.

Accordingly, the antisense oligonucleotides in accordance with this invention can comprise from about 10 to about 100 nucleotides, including, but not limited to about 10, 11, 12, 13, 14, 15, 16, 17, 18, 19, 20, 21, 22, 23, 24, 25, 30, 40, 50, 60, 70, 80, 90, or about 100 nucleotides.

Antisense nucleic acids can be produced by standard techniques (see, for example, Shewmaker et al., U.S. Pat. No. 5,107,065). Appropriate oligonucleotides can be designed using OLIGO software (Molecular Biology Insights, Inc., Cascade, Colo.; http://www.oligo.net).

Those of ordinary skill in the art will readily appreciate that a silencing element can be prepared according to any available technique including, but not limited to, chemical synthesis, enzymatic or chemical cleavage in vivo or in vitro, template transcription in vivo or in vitro, or combinations of the foregoing.

As discussed above, the silencing elements employed in the methods and compositions of the invention can comprise a DNA molecule which when transcribed produces an interfering RNA or a precursor thereof, or an antisense oligonucleotide. In such embodiments, the DNA molecule encoding the silencing element is found in an expression cassette. In addition, polynucleotides that comprise a coding sequence for a polypeptide of interest are found in an expression cassette.

The expression cassette comprises one or more regulatory sequences, selected on the basis of the cells to be used for expression, operably linked to a polynucleotide encoding the silencing element or polypeptide of interest. “Operably linked” is intended to mean that the nucleotide sequence of interest (i.e., a DNA encoding a silencing element or a coding sequence for a polypeptide of interest) is linked to the regulatory sequence(s) in a manner that allows for expression of the nucleotide sequence (e.g., in an in vitro transcription/translation system or in a cell when the expression cassette or vector is introduced into a cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, California). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression cassette can depend on such factors as the choice of the host cell to be transformed, the level of expression of the silencing element or polypeptide of interest desired, and the like. Such expression cassettes typically include one or more appropriately positioned sites for restriction enzymes, to facilitate introduction of the nucleic acid into a vector.

It will further be appreciated that appropriate promoter and/or regulatory elements can readily be selected to allow expression of the relevant transcription units/silencing elements in the cell of interest. Promoters can be constitutively active, chemically-inducible, development-, cell-, or tissue-specific promoters. In certain embodiments, the promoter utilized to direct intracellular expression of a silencing element is a promoter for RNA polymerase III (Pol III). References discussing various Pol III promoters, include, for example, Yu et al. (2002) Proc. Natl. Acad. Sci. 99(9), 6047-6052; Sui et al. (2002) Proc. Natl. Acad. Sci. 99(8), 5515-5520 (2002); Paddison et al. (2002) Genes and Dev. 16, 948-958; Brummelkamp et al. (2002) Science 296, 550-553; Miyagashi (2002) Biotech. 20, 497-500; Paul et al. (2002) Nat. Biotech. 20, 505-508; Tuschl et al. (2002) Nat. Biotech. 20, 446-448. According to other embodiments, a promoter for RNA polymerase I, e.g., a tRNA promoter, can be used. See McCown et al. (2003) Virology 313(2):514-24; Kawasaki (2003) Nucleic Acids Res. 31 (2):700-7. In some embodiments in which the polynucleotide comprises a coding sequence for a polypeptide of interest, a promoter for RNA polymerase II can be used.

The regulatory sequences can also be provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

In vitro transcription can be performed using a variety of available systems including the T7, SP6, and T3 promoter/polymerase systems (e.g., those available commercially from Promega, Clontech, New England Biolabs, and the like) in order to make a silencing element. Vectors including the T7, SP6, or T3 promoter are well known in the art and can readily be modified to direct transcription of silencing elements. When silencing elements are synthesized in vitro, the strands can be allowed to hybridize before introducing into a cell or before administration to a subject. As noted above, silencing elements can be delivered or introduced into a cell as a single RNA molecule including self-complementary portions (e.g., an shRNA that can be processed intracellularly to yield an siRNA), or as two strands hybridized to one another. In other embodiments, the silencing elements employed are transcribed in vivo. As discussed elsewhere herein, regardless of whether the silencing element is transcribed in vivo or in vitro, in either scenario, a primary transcript can be produced which can then be processed (e.g., by one or more cellular enzymes) to generate the interfering RNA that accomplishes gene inhibition.

In those embodiments in which the silencing element is an interfering RNA, the interfering RNA can be generated by transcription from a promoter, either in vitro or in vivo. For instance, a construct can be provided containing two separate transcribable regions, each of which generates a 21-nt transcript containing a 19-nt region complementary with the other. Alternatively, a single construct can be utilized that contains opposing promoters and terminators positioned so that two different transcripts, each of which is at least partly complementary to the other, are generated. Alternatively, an RNA-inducing agent can be generated as a single transcript, for example by transcription of a single transcription unit encoding self complementary regions. A template is employed that includes first and second complementary regions, and optionally includes a loop region connecting the portions. Such a template can be utilized for in vitro transcription or in vivo transcription, with appropriate selection of promoter and, optionally, other regulatory elements, e.g., a terminator.

In some embodiments, the expression cassette or polynucleotide can comprise sequences sufficient for site-specific integration into the genome of the cell to which is has been introduced.

In some embodiments, the presently disclosed delivery system complexes comprise a liposome encapsulating a nano-precipitate that is a polypeptide of interest that is to be delivered to a cell. The delivery system complexes disclosed herein are capable of introducing a polypeptide into the intracellular region of a cell.

In some of these embodiments, the polypeptide that is delivered into the cell comprises a cationic or an anionic polypeptide. As used herein, an “anionic polypeptide” is a polypeptide as described herein that has a net negative charge at physiological pH. The anionic polypeptide can comprise at least about 20%, 30%, 40%, 50%, 60%, 70%, 75%, 80%, 85%, 90%, 95%, 96%, 97%, 98%, 99%, or 100% amino acid residues that have a negative charge at physiological pH. These include aspartic acid (D), asparagine (N), glutamic acid (E), and glutamine (Q). In particular embodiments, the polypeptide of interest is acetylated at the amino and/or carboxyl termini to enhance the negative charge of the polypeptide. In certain embodiments, the polypeptide is phosphorylated (i.e., comprises at least one phosphate group). Alternatively, a “cationic polypeptide” is a polypeptide as described herein that has a net positive charge at physiological pH. The cationic polypeptide can comprise at least about 20%, 30%, 40%, 50%, 60%, 70%, 75%, 80%, 85%, 90%, 95%, 96%, 97%, 98%, 99%, or 100% amino acid residues that have a positive charge at physiological pH. These include lysine (K), arginine (R), and histidine (H).

In some of the embodiments wherein the delivery system complex comprises a polypeptide of interest, the polypeptide of interest has at least about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, or more amino acid residues. In some embodiments, the polypeptide of interest that is delivered to a cell using the delivery system complexes disclosed herein can have a molecular weight from about 200 Daltons to about 50,000 Daltons, including but not limited to, about 200, 300, 400, 500, 600, 700, 800, 900, 1,000, 5,000, 10,000, 20,000, 30,000, 40,000, and 50,000 Daltons. In particular embodiments, the delivery system complex is capable of delivering between about 1 and about 2×10¹⁶ molecules of the polypeptide of interest in a single lipid vehicle, including but not limited to about 1, 10, 100, 500, 1000, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, and 2×10¹⁶ molecules.

In some embodiments, the polypeptide of interest has an amino acid sequence that mimics the catalytic domain of an enzyme that functions in an essential signaling pathway in the cell (e.g., EGFR). A non-limiting example of such an enzyme is the epidermal growth factor receptor (EGFR) tyrosine kinase. The polypeptide of interest can therefore comprise the EV peptide (set forth as SEQ ID NO: 3) described in International Application No. PCT/US2009/042485, entitled “Methods and compositions for the delivery of bioactive compounds” that was filed on May 1, 2009, and is herein incorporated by reference in its entirety. In other embodiments wherein the delivery system complex comprises a polypeptide of interest, the polypeptide of interest comprises an imaging peptide comprising at least one caspase 3 recognition motif, as described in International. Appl. No. PCT/US2009/042485. As further described in International. Appl. No. PCT/US2009/042485, in some of these embodiments, the delivery system complex further comprises a cytotoxic bioactive compound.

It should be noted that the delivery system complexes can comprise more than one type of bioactive compound.

III. PEGylated Delivery Systems and Targeted Delivery Systems

As described elsewhere herein, the delivery system complexes can have a surface charge (e.g., positive charge). In some embodiments, the surface charge of the liposome of the delivery system can be minimized by incorporating lipids comprising polyethylene glycol (PEG) moieties into the liposome. Reducing the surface charge of the liposome of the delivery system can reduce the amount of aggregation between the delivery system complexes and serum proteins and enhance the circulatory half-life of the complex (Yan, Scherphof, and Kamps (2005) J Liposome Res 15:109-139). Thus, in some embodiments, the exterior surface of the liposome or the outer leaflet of the lipid bilayer of the delivery system comprises a PEG molecule. Such a complex is referred to herein as a PEGylated delivery system complex. In these embodiments, the outer leaflet of the lipid bilayer of the liposome of the delivery system complex comprises a lipid-PEG conjugate.

A PEGylated delivery system complex can be generated through the post-insertion of a lipid-PEG conjugate into the lipid bilayer through the incubation of the delivery system complex with micelles comprising lipid-PEG conjugates, as known in the art and described elsewhere herein (Ishida et al. (1999) FEBS Lett. 460:129-133; Perouzel et al. (2003) Bioconjug. Chem. 14:884-898; see Experimental section). By “lipid-polyethylene glycol conjugate” or “lipid-PEG conjugate” is intended a lipid molecule that is covalently bound to at least one polyethylene glycol molecule. In some embodiments, the lipid-PEG conjugate comprises 1,2-distearoyl-sn-glycero-3-phosphoethanolamine-N-carboxy-polyethylene glycol (DSPE-PEG). As described immediately below, these lipid-PEG conjugates can be further modified to include a targeting ligand, forming a lipid-PEG-targeting ligand conjugate (e.g., DSPE-PEG-AA). The term “lipid-PEG conjugate” also refers to these lipid-PEG-targeting ligand conjugates and a delivery system complex comprising a liposome comprising a lipid-PEG targeting ligand conjugate are considered to be both a PEGylated delivery system complex and a targeted delivery system complex, as described immediately below.

Alternatively, the delivery system complex can be PEGylated through the addition of a lipid-PEG conjugate during the formation of the outer leaflet of the lipid bilayer.

PEGylation of liposomes enhances the circulatory half-life of the liposome by reducing clearance of the complex by the reticuloendothelial (RES) system. While not being bound by any particular theory or mechanism of action, it is believed that a PEGylated delivery system complex can evade the RES system by sterically blocking the opsonization of the complexes (Owens and Peppas (2006) Int J Pharm 307:93-102). In order to provide enough steric hindrance to avoid opsonization, the exterior surface of the liposome must be completely covered by PEG molecules in the “brush” configuration. At low surface coverage, the PEG chains will typically have a “mushroom” configuration, wherein the PEG molecules will be located closer to the surface of the liposome. In the “brush” configuration, the PEG molecules are extended further away from the liposome surface, enhancing the steric hindrance effect. However, over-crowdedness of PEG on the surface may decrease the mobility of the polymer chains and thus decrease the steric hindrance effect (Owens and Peppas (2006) Int J Pharm 307:93-102).

The conformation of PEG depends upon the surface density and the molecular mass of the PEG on the surface of the liposome. The controlling factor is the distance between the PEG chains in the lipid bilayer (D) relative to their Flory dimension, R_{F}, which is defined as aN^{3/5}, wherein a is the persistence length of the monomer, and N is the number of monomer units in the PEG (see Nicholas et al. (2000) Biochim Biophys Acta 1463:167-178, which is herein incorporated by reference). Three regimes can be defined: (1) when D>2 R_{F} (interdigitated mushrooms); (2) when D<2 R_{F} (mushrooms); and (3) when D<R_{F} (brushes) (Nicholas et al.).

In certain embodiments, the PEGylated delivery system complex comprises a stealth delivery system complex. By “stealth delivery system complex” is intended a delivery system complex comprising a liposome wherein the outer leaflet of the lipid bilayer of the liposome comprises a sufficient number of lipid-PEG conjugates in a configuration that allows the delivery system complex to exhibit a reduced uptake by the RES system in the liver when administered to a subject as compared to non PEGylated delivery system complexes. RES uptake can be measured using assays known in the art, including, but not limited to the liver perfusion assay described in International Application No. PCT/US2009/042485, filed on May 1, 2009. In some of these embodiments, the stealth delivery system complex comprises a liposome, wherein the outer leaflet of the lipid bilayer of the liposome comprises PEG molecules, wherein said D<R_{F}.

In some of those embodiments in which the PEGylated delivery system is a stealth polynucleotide system, the outer leaflet of the lipid bilayer of the cationic liposome comprises a lipid-PEG conjugate at a concentration of about 4 mol % to about 15 mol % of the outer leaflet lipids, including, but not limited to, about 4 mol %, about 5 mol %, about 6 mol %, about 7 mol %, 8 mol %, about 9 mol %, about 10 mol %, about 11 mol %, about 12 mol %, about 13 mol %, about 14 mol %, and about 15 mol % PEG. In certain embodiments, the outer leaflet of the lipid bilayer of the cationic liposome of the stealth delivery system complex comprises about 10.6 mol % PEG. Higher percentage values (expressed in mol %) of PEG have also surprisingly been found to be useful. Useful mol % values include those from about 12 mol % to about 50 mol %. Preferably, the values are from about 15 mol % to about 40 mol %. Also preferred are values from about 15 mol % to about 35 mol %. Most preferred values are from about 20 mol % to about 25 mol %, for example 23 mol %.

The polyethylene glycol moiety of the lipid-PEG conjugate can have a molecular weight ranging from about 100 to about 20,000 g/mol, including but not limited to about 100 g/mol, about 200 g/mol, about 300 g/mol, about 400 g/mol, about 500 g/mol, about 600 g/mol, about 700 g/mol, about 800 g/mol, about 900 g/mol, about 1000 g/mol, about 5000 g/mol, about 10,000 g/mol, about 15,000 g/mol, and about 20,000 g/mol. In some embodiments, the lipid-PEG conjugate comprises a PEG molecule having a molecular weight of about 2000 g/mol. In certain embodiments, the lipid-PEG conjugate comprises DSPE-PEG₂₀₀₀.

In some embodiments, the delivery system complex comprises a liposome, wherein the exterior surface of the liposome, or the delivery system complex comprises a lipid bilayer wherein the outer leaflet of the lipid bilayer, comprises a targeting ligand, thereby forming a targeted delivery system. In these embodiments, the outer leaflet of the liposome comprises a targeting ligand. By “targeting ligand” is intended a molecule that targets a physically associated molecule or complex to a targeted cell or tissue. As used herein, the term “physically associated” refers to either a covalent or non-covalent interaction between two molecules. A “conjugate” refers to the complex of molecules that are covalently bound to one another. For example, the complex of a lipid covalently bound to a targeting ligand can be referred to as a lipid-targeting ligand conjugate.

Alternatively, the targeting ligand can be non-covalently bound to a lipid. “Non-covalent bonds” or “non-covalent interactions” do not involve the sharing of pairs of electrons, but rather involve more dispersed variations of electromagnetic interactions, and can include hydrogen bonding, ionic interactions, Van der Waals interactions, and hydrophobic bonds.

Targeting ligands can include, but are not limited to, small molecules, peptides, lipids, sugars, oligonucleotides, hormones, vitamins, antigens, antibodies or fragments thereof, specific membrane-receptor ligands, ligands capable of reacting with an anti-ligand, fusogenic peptides, nuclear localization peptides, or a combination of such compounds. Non-limiting examples of targeting ligands include asialoglycoprotein, insulin, low density lipoprotein (LDL), folate, benzamide derivatives, peptides comprising the arginine-glycine-aspartate (RGD) sequence, and monoclonal and polyclonal antibodies directed against cell surface molecules. In some embodiments, the small molecule comprises a benzamide derivative. In some of these embodiments, the benzamide derivative comprises anisamide.

The targeting ligand can be covalently bound to the lipids comprising the liposome or lipid bilayer of the delivery system, including a cationic lipid, or a co-lipid, forming a lipid-targeting ligand conjugate. As described above, a lipid-targeting ligand conjugate can be post-inserted into the lipid bilayer of a liposome using techniques known in the art and described elsewhere herein (Ishida et al. (1999) FEBS Lett. 460:129-133; Perouzel et al. (2003) Bioconjug. Chem. 14:884-898; see Experimental section). Alternatively, the lipid-targeting ligand conjugate can be added during the formation of the outer leaflet of the lipid bilayer.

Some lipid-targeting ligand conjugates comprise an intervening molecule in between the lipid and the targeting ligand, which is covalently bound to both the lipid and the targeting ligand. In some of these embodiments, the intervening molecule is polyethylene glycol (PEG), thus forming a lipid-PEG-targeting ligand conjugate. An example of such a lipid-targeting conjugate is DSPE-PEG-AA, in which the lipid 1,2-distearoyl-sn-glycero-3-phosphoethanolamine-N-carboxyl (DSPE) is bound to polyethylene glycol (PEG), which is bound to the targeting ligand anisamide (AA). Thus, in some embodiments, the cationic lipid vehicle of the delivery system comprises the lipid-targeting ligand conjugate DSPE-PEG-AA.

By “targeted cell” is intended the cell to which a targeting ligand recruits a physically associated molecule or complex. The targeting ligand can interact with one or more constituents of a target cell. The targeted cell can be any cell type or at any developmental stage, exhibiting various phenotypes, and can be in various pathological states (i.e., abnormal and normal states). For example, the targeting ligand can associate with normal, abnormal, and/or unique constituents on a microbe (i.e., a prokaryotic cell (bacteria), viruses, fungi, protozoa or parasites) or on a eukaryotic cell (e.g., epithelial cells, muscle cells, nerve cells, sensory cells, cancerous cells, secretory cells, malignant cells, erythroid and lymphoid cells, stem cells). Thus, the targeting ligand can associate with a constitutient on a target cell which is a disease-associated antigen including, for example, tumor-associated antigens and autoimmune disease-associated antigens. Such disease-associated antigens include, for example, growth factor receptors, cell cycle regulators, angiogenic factors, and signaling factors.

In some embodiments, the targeting ligand interacts with a cell surface protein on the targeted cell. In some of these embodiments, the expression level of the cell surface protein that is capable of binding to the targeting ligand is higher in the targeted cell relative to other cells. For example, cancer cells overexpress certain cell surface molecules, such as the HER2 receptor (breast cancer) or the sigma receptor. In certain embodiments wherein the targeting ligand comprises a benzamide derivative, such as anisamide, the targeting ligand targets the associated delivery system complex to sigma-receptor overexpressing cells, which can include, but are not limited to, cancer cells such as small- and non-small-cell lung carcinoma, renal carcinoma, colon carcinoma, sarcoma, breast cancer, melanoma, glioblastoma, neuroblastoma, and prostate cancer (Aydar, Palmer, and Djamgoz (2004) Cancer Res. 64:5029-5035).

Thus, in some embodiments, the targeted cell comprises a cancer cell. The terms “cancer” or “cancerous” refer to or describe the physiological condition in mammals that is typically characterized by unregulated cell growth. As used herein, “cancer cells” or “tumor cells” refer to the cells that are characterized by this unregulated cell growth. The term “cancer” encompasses all types of cancers, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer. In some embodiments, the targeted cancer cell comprises a lung cancer cell. The term “lung cancer” refers to all types of lung cancers, including but not limited to, small cell lung cancer (SCLC), non-small-cell lung cancer (NSCLC, which includes large-cell lung cancer, squamous cell lung cancer, and adenocarcinoma of the lung), and mixed small-cell/large-cell lung cancer. In particular, the nanoparticles are for use against melanomas.

### IV. Liposome-Encapsulated Nano-Precipitated Bioactive Compounds-Neighboring Effect and Enhanced Anticancer Efficacy

Encapsulation of cisplatin (CDDP) into nanoparticles (NPs) with high drug loading and encapsulation efficiency has been previously unachievable due to the poor solubility of CDDP. However, this barrier has been overcome with a reverse microemulsion method appropriating CDDP' s poor solubility advantageously by promoting the synthesis of a pure cisplatin nanoparticle with a high drug loading capacity (approximately 80.8wt %). Actively targeted CDDP NPs exhibit significant accumulation in human A375M melanoma tumor cells in vivo. In addition, CDDP NPs achieve potent anti-tumor efficacy through the neighboring effect at a dose of 1 mg/kg which is an observation made in vivo when the tumor cells that took up CDDP NPs released active drug following apoptosis. Via diffusion, surrounding cells that are previously unaffected showed intake of the released drug and their apoptosis soon follow. This observation is also made in vitro when A375M melanoma tumor cells incubated with CDDP NPs exhibited release of active drug and induce apoptosis on untreated neighboring cells. However, the neighboring effect was unique to rapidly proliferating tumor cells. Liver functional parameters and H&E staining of liver tissue in vivo fail to detect any difference between CDDP NP treated and control groups in terms of tissue health. By simultaneously promoting an increase in cytotoxicity and less side effects over free CDDP, CDDP NPs show great therapeutic potential with lower doses of drug while enhancing anti-cancer effectiveness.

The use of cisplatin (CDDP) as a cytotoxic drug was pioneered by Rosenberg while studying the effects of electrical fields on the growth of bacteria.⁹² CDDP has become a first-line therapy against a wide spectrum of solid neoplasms, including bladder, ovarian, colorectal and melanoma cancers.^{93, 94} However, drug resistance and related systemic toxicities (e.g. nephro- and neuro-toxicities) limit the clinical use of CDDP.^{95, 96}

Formulating small molecule drugs into nanoparticles (NPs), such as liposomal or polymeric formulations allows for a significant reduction of adverse side effects while maintaining anti-tumor efficacy. Therefore, this class of nanomedicine is currently established as the cutting edge method in treating a variety of cancers.^{97, 98} With modification, NPs are able to avoid undesired uptake by the reticuloendothelial system (RES) and improve circulation of their encapsulated drugs in the blood compared to free drug.⁹⁹ Thus, drug efficacy can be greatly increased without a subsequent increase in collateral damage to healthy tissues.

Similarly, uptake of NPs by tumor cells can be mediated by tumor targeting ligands, such as aptamer,⁹ RGD peptide and anisamide (AA).¹⁰⁰⁻¹⁰³ The accumulation of nano-sized formulations in tumors is also highly dependent on the enhanced permeability and retention (EPR) effect due to the disorganized and tortuous tumor endothelium.¹⁰⁴ Nonetheless, the accessibility of NPs into tumor cells primarily depends on the properties of the NPs, especially size. NPs with a diameter less than 50 nm can penetrate deeper into poorly permeable, hypo-vascular tumors with greater efficiency than larger NPs.^{105, 106}

However, the poor solubility of inorganic CDDP in both water and oil significantly limits the development of NPs with high drug loading and encapsulation efficacy. In a previous study, lipid-coated CDDP (LPC) NPs composed entirely of CDDP and outer leaflet lipids were successfully synthesized and characterized with high drug loading capacity. Compared to nanocapsules,¹⁰⁷⁻¹⁰⁹ LPC NPs were formulated via a reverse microemulsion method appropriating a mixture of two emulsions containing KCl and a highly soluble precursor of CDDP, cis-diaminedihydroplatinum (II). The CDDP NPs were first stabilized for dispersion in an organic solvent by coating with 1,2-dioleoyl-sn-glycero-3-phosphate (DOPA). After purification, additional lipids were added to stabilize the NPs for dispersion in an aqueous solution. The final NPs contain a lipid bilayer coating and are named Lipid-Pt—Cl (LPC) NPs.

The anticancer efficacy of LPC NPs on A375M melanoma xenograft tumors is evaluated herein. Furthermore, the in vitro release profile of LPC NPs in cells incubated in a medium with 50% fetal bovine serum is evaluated. Also, the diffusion and distance dependent neighboring effect of LPC NPs are examined both in vitro and in vivo. Finally, the biodistribution and safety profile of LPC NPs are also determined.

i) Physiochemical Characterizations of LPC NPs

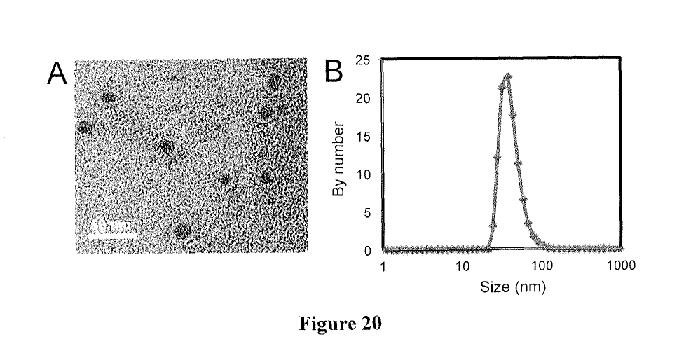

While the major side effects of CDDP can be minimized through the usage of NPs for drug delivery, the poor solubility of CDDP has hampered the development of a successful nanoparticulate formulation. In some embodiments, lipid-coated, platinum-filled drug formulations (LPC NPs) characterized with a core of CDDP and 80 wt % of drug loading are synthesized. In some of these embodiments, LPC NPs are negatively stained with uranyl acetate for transmission electron microscopy (TEM). The images reveal the core/membrane nanostructure of NPs with a size of approximately 20 nm in diameter (FIG. 20a). DLS results (FIG. 20b) further indicate that the hydrodynamic diameter of NPs was approximately 30 nm, slightly larger than the diameter observed in TEM images. The drug loading capacity of LPC NPs as determined by using inductively coupled plasma mass spectrometry (ICP-MS) is 80.8wt %. Other liposomal formulations of CDDP based drugs, such as SPI-77 (6.7wt %) and Lipoplatin (10 wt %), which are either in phase II clinical trials or clinically approved respectively, cannot achieve such high drug loading.¹¹⁰

ii) LPC NPs Deliver CDDP Efficiently Into A375M Cells and Show Significant Efficacy

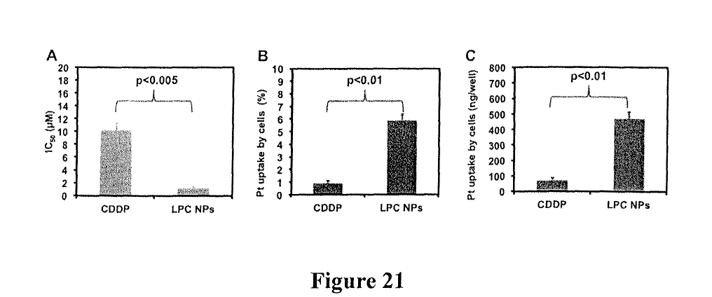

To test the anticancer efficacy of LPC NPs, the cytotoxicity of LPC NPs in A375M melanoma cancer cells was evaluated. As shown in FIG. 21a, the LPC NPs showed a nearly ten-fold lower IC₅₀ than free drug (1.2 v.s. 10.2 μM) in the growth inhibition in A375M cells. The control empty liposome vesicles did not induce any cytotoxicity (data not shown). FIGS. 21b and c quantitatively present cellular uptake of NPs measured using ICP-MS. As indicated, LPC NPs deliver CDDP efficiently into A375M cells with a 6.5 fold increase in internalized drug over free CDDP. In vitro studies illustrated that LPC NPs efficiently transport CDDP into cells and result in a significantly lower IC₅₀ over free CDDP.

iii) LPC NPs Show High Accumulation of CDDP in A375M Xenograft Bearing Mice and Significant Anti-Tumor Efficacy at a Low Dose

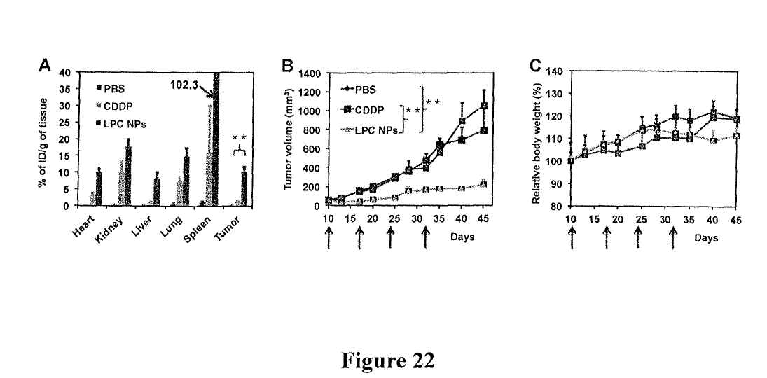

The biodistribution of free CDDP and LPC NPs in tumor-bearing mice was compared. Twenty-four hours post-IV injection, 10.5% of the injected dose per gram of LPC NPs is accumulated in the tumors, which is significantly higher than the 1.2% of the injected dose per gram of free CDDP (FIG. 22a). To determine the efficacy of LPC NPs in treating A375M tumors, the drugs were administered weekly by IV injection at a dose of 1.0 mg/kg Pt. LPC NPs inhibit the growth of A375M tumors significantly without reducing the body weight of the treated animals (FIGS. 22b and c). However, free CDDP at the same dose and dosing schedule was ineffective, possibly due to a low accumulation in the tumors.

In vivo, the small size of LPC NPs facilitat the accumulation of LPC NPs in tumor cells through the EPR effect. Therefore, LPC NPs achieved an accumulation of 10.5% injected dose (ID)/g in A375M tumor cells and exhibit significant anticancer therapeutic effect at a low dose and generous dosing schedule while free CDDP was ineffective at the same dose. LPC NPs are therefore capable of inducing considerable anti-tumor efficacy at a significantly lower dose than free CDDP and can be applied to treat a wide range of cancers.

iv) LPC NPs Induced Discernible Apoptosis in A375M Tumors

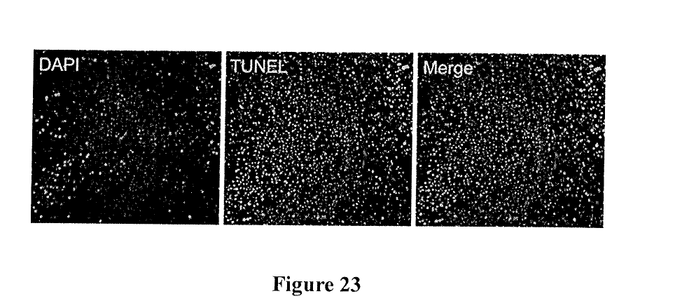

After confirming that LPC NPs showed significant antitumor efficacy, an additional experiment was used to evaluate their efficacy in treating large tumors. Mice bearing A375M melanoma tumors of approximately 600 mm³ were dosed with IV administrations of LPC NPs at a dose of 3.0 mg/kg Pt once a week, for a period of two weeks. One week after the final injection, the mice were sacrificed and the tumors were assayed using TUNEL, a marker of apoptosis. As shown in FIG. 23, about 90% of tumor cells are apoptotic, resulting in a 60% reduction in tumor volume (data not shown). It may not be the case that NPs can reach 90% of tumor cells and additional mechanisms may be contributing to the tumor reduction. The majority of apoptosis may actually be induced by the small fraction of cells that took up the NPs in a pattern known as the neighboring effect. This phenomenon is characterized as the uptake of NPs by tumor cells that become in situ drug depots and release active drugs to induce apoptosis in surrounding cells. As such, the neighboring effect is a distance and diffusion dependent effect.

v) Neighboring Effect Contributed to Significant In Vivo Apoptosis

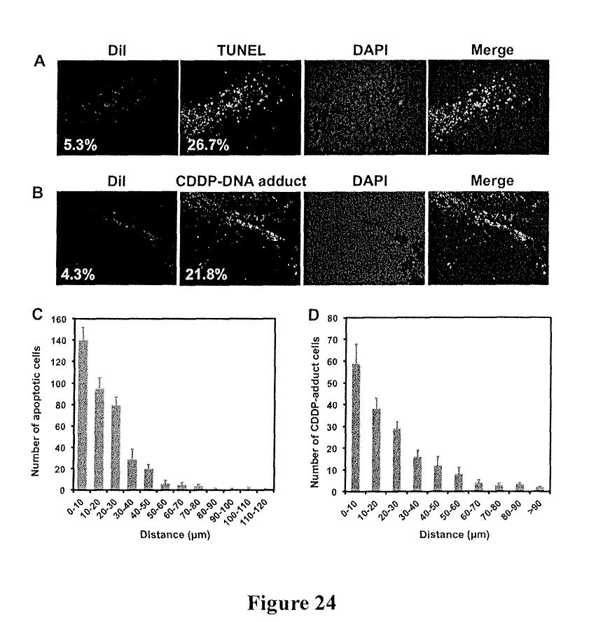

The intracellular distribution of LPC NPs in tumors was investigated and tested the apoptosis of tumor cells using TUNEL and CDDP-DNA adduct antibody. To determine the mechanism of the neighboring effect, a lipophilic dye, 1,1′-Dioctadecyl-3,3,3′,3′-tetramethylindocarbocyanine perchlorate (DiI) is used to label LPC NPs. DiI was entrapped in an asymmetric bilayer. Results indicate that only 5.3% of the tumor cells took up the NPs and yet, 26.7% of cells underwent apoptosis (FIG. 24a). While it is possible that the amount of NPs in some TUNEL positive cells was too low to be detected because of the detection limitations of the technique, a “nearest neighbor” analysis is used to eliminate this possibility. The number of apoptotic cells was measured as a function of the distance to the nearest DiI positive cells. In groups treated with LPC NPs, the large number of green cells (TUNEL positive) close to red cells (DiI positive) gradually decay to a small number of green cells far from red cells (FIG. 24c). The data therefore indicate that the neighboring effect is indeed facilitated by diffusion and varies with distance from the depot cell.

In addition, an antibody specific to the Pt-DNA adduct was used in an assay for a nearest neighbor analysis to determine if Pt-DNA adduct is the cause of cell death.¹¹¹ As shown in FIG. 24b, the formation of CDDP-DNA adducts was confirmed. It was consistently observed that a relatively small number of DiI-positive cells were able to induce the formation of the CDDP-DNA adduct in a large number of surrounding cells (FIGS. 24c and d). Therefore, formation of CDDP-DNA adduct was directly attributed to the release of CDDP in vivo. This data further provides strong evidence for the neighboring effect by suggesting that active Pt drugs released from dead or dying depot cells are diffused into previously unaffected cells.

vi) In Vitro and Intracellular Release of Drugs from NPs and Cytotoxicity Assays

To test the neighboring effect in vitro, intracellular release of CDDP from LPC

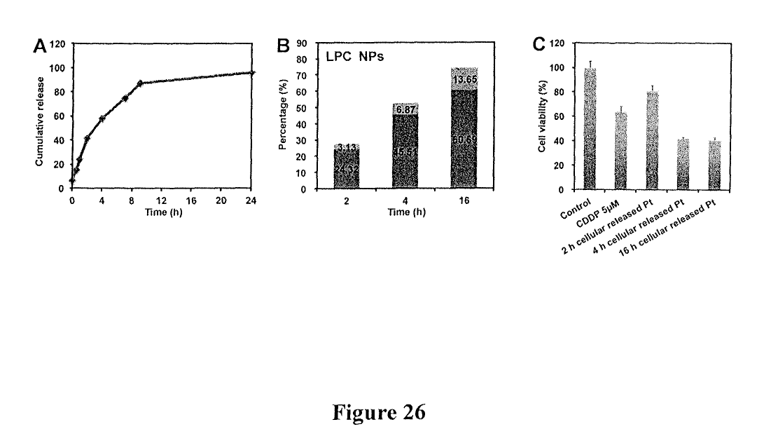

NP was investigated. The kinetics regarding the release of platinum-based drugs from LPC NPs was evaluated in 50% FBS medium at 37° C. As shown in FIG. 26a, LPC NPs exhibited a sustained release of Pt over time with a half-life of 3.0 h.

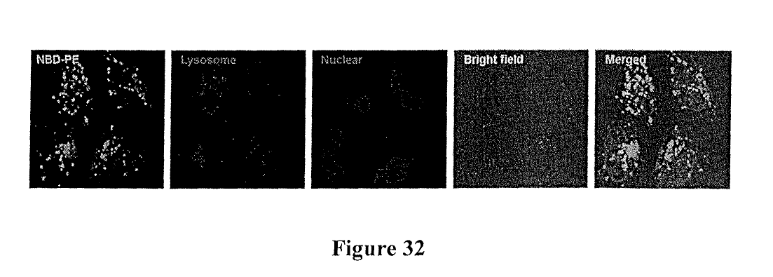

The NPs were labeled using fluorescent NBD-PE lipid and incubated them with cells. Some of the nanoparticles are co-localized with lysosomes as indicated by yellow spots (FIG. 32). However, a large number of the NPs taken into the tumor cells do not co-localize with lysosomes.

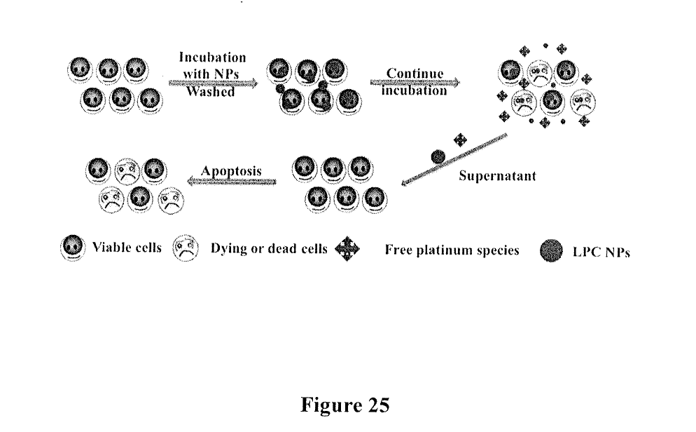

The neighboring effect in vitro was tested using the procedure shown in FIG. 25. By culturing untreated cells with medium from LPC NPs treated cells, the activity of released CDDP was tested. Cells were first incubated with LPC NPs for 2, 4 or 16 h and subsequently washed and cultured. At different time points, the released NPs and free drugs in the medium were separated by centrifugation at 16,000g for 20 min. After centrifugation, it was observed that the LPC NPs exhibited cellular release and that free drugs composed a major fraction of the medium (FIG. 26b). To test the activity of drugs released from cells which previously entrapped NPs, the medium collected at different time points was transferred and incubated with untreated cells. After 48 h, the viability of the tumor cells was assayed using MTS. As shown in FIG. 26c, the medium containing more drugs is more toxic.

vii) Study of the Neighboring Effect In Vitro

In addition, the neighboring effect was further investigated using a common protocol. A375M-GFP cells that stably expressed green-fluorescence protein (green) are treated with 50 tM of LPC NPs for 4 h, washed, and mixed with untreated A375M cells at a 1:10 ratio. Cells were incubated for an additional 24 or 48 h. Then, cell apoptosis was examined with Alexa Fluor 568-labeled Annexin V (red), an apoptosis marker.

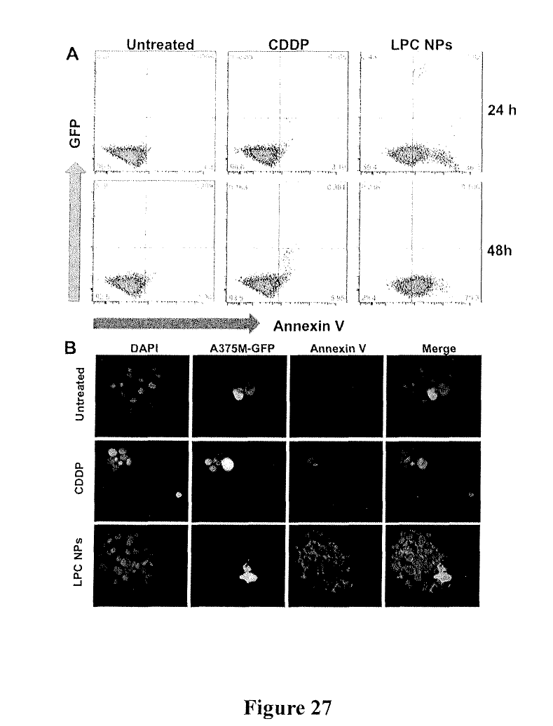

In FIGS. 27a and b, many cells that are near the green, LPC NP-treated cells were undergoing apoptosis. Groups treated with LPC NPs exhibit a pronounced effect while cells treated with CDDP show only minimal signs of the neighboring effect. The apoptosis results were further quantified using flow cytometry. Cells were analyzed at 24 (upper panels) or 48 (lower panels) h (FIG. 27a). Untreated cells served as the control;

after 24 or 48 h, A375M-GFP cells survive, while cells treated with CDDP die and disappear at both time points. Furthermore, at 24 or 48 h the CDDP-treated cells do not induce significant apoptosis in the unlabeled and untreated cells. At 48 h, less than 6% of untreated cells are apoptotic. In contrast, cells treated with LPC NPs induce a higher percent of apoptotic cells, which are not directly exposed to CDDP. At 48 h, few green cells are left in both cases, but 70% apoptotic cells appear in the untreated cell population for LPC NPs. This demonstrated that CDDP released from dead or dying cells is able to induce apoptosis on untreated tumor cells. These results confirm that the neighboring effect as characterized by the release of active drug from dead or dying cells after NP internalization and subsequent apoptosis in previously unaffected cells is validated both in vivo and in vitro. The cells transfected with NPs do in fact, serve as drug depots and affect the untreated cells in a manner dependent on distance and diffusion.

viii) Safety Evaluations LPC NPs are Safe and No Neighboring Effect is Observed in Major Organs

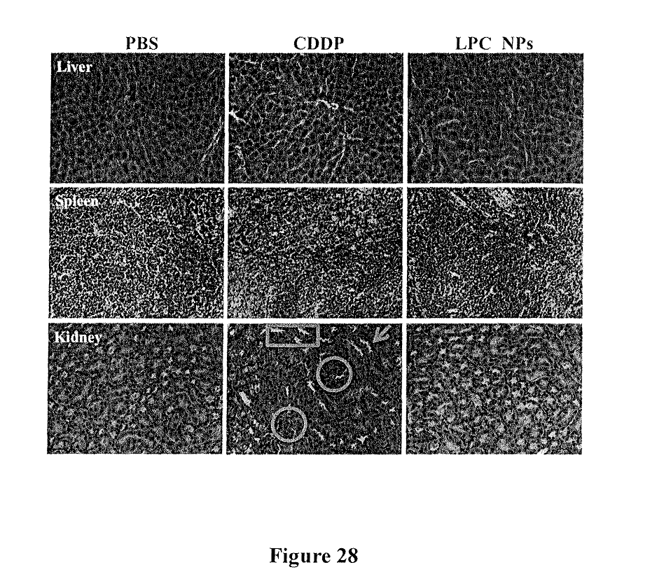

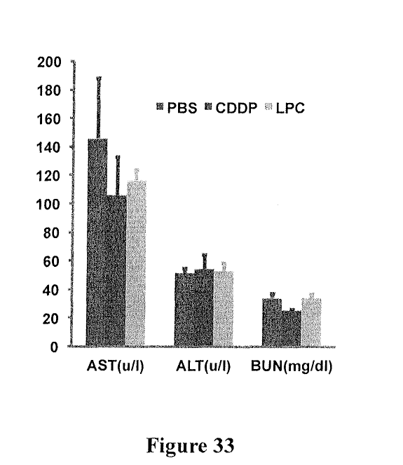

Although the neighboring effect displayed profound effects against rapidly proliferating tumor cells, its potential toxicity toward normal organs is of concern. Therefore, mechanism of the neighboring effect in normal tissues was studied. Since the liver is characterized as the major organ affecting clearance of NPs, the functional parameters aspartate transaminase (AST) and aspartate aminotransferase (ALT) of liver cells treated with free CDDP or LPC NPs were studied. The data indicate that the AST and ALT functional parameters from mice treated with CDDP and LPC NPs fall within the normal range (FIG. 33). Furthermore, the comparison between H&E stained liver cells treated with LPC NPs and PBS display negligible differences in morphology (FIG. 28). Therefore, LPC NPs only posed a minimal threat to normal liver function, which is probably due to the strong repair ability of cisplatin-induced DNA damage in the liver.¹¹²⁻¹¹⁴

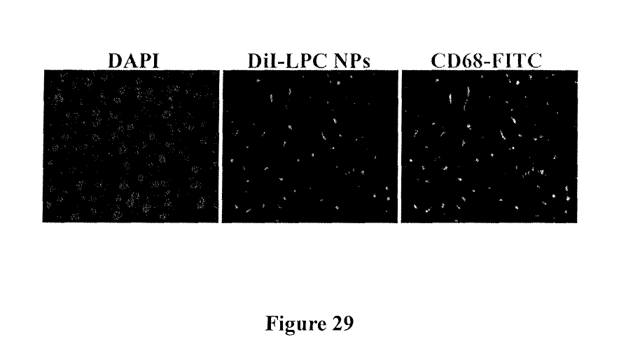

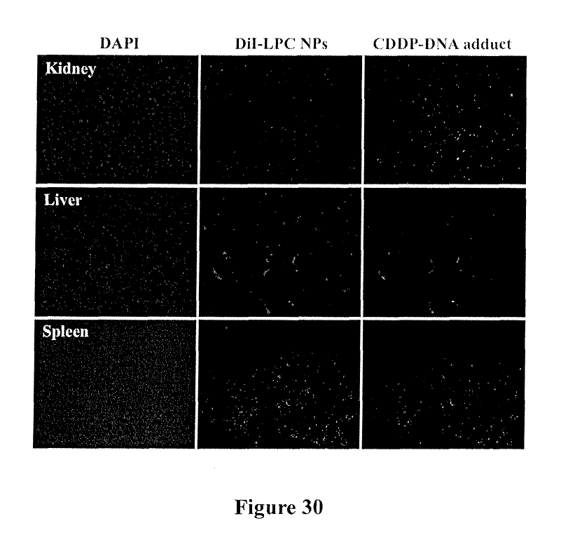

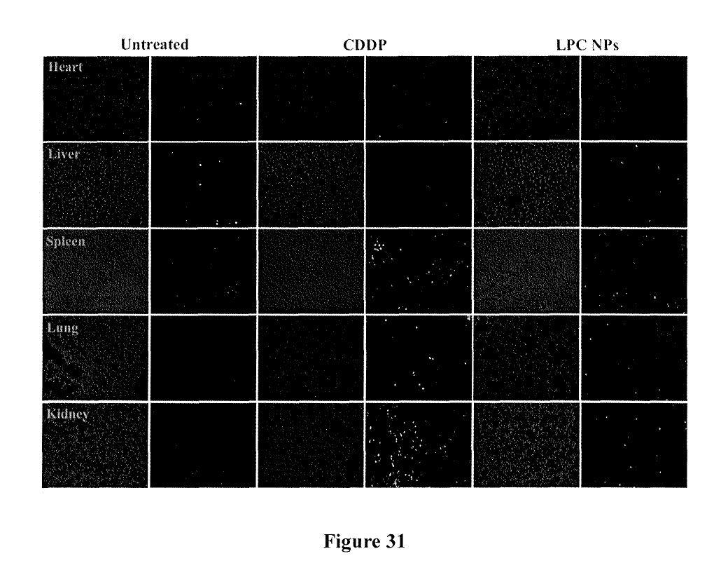

In addition, it is shown that Kupffer cells are responsible for harmlessly removing most of the NPs in the liver (FIG. 29) while hepatocytes show minimal LPC NPs uptake. Therefore, while the formation of the CDDP-DNA adduct was observed in some liver cells (FIG. 30), subsequent apoptosis was not noted (FIG. 31). This observation could be due to the successful repair of CDDP-DNA adducts.¹¹²⁻¹¹⁴ This pattern is also found in other critical organs such as the kidney, spleen, heart, and lung.

Because the spleen is responsible for significant NP uptake (FIG. 22a), histological analysis of the spleen was also performed to exclude any spleen toxicity induced by the NPs (FIG. 28). Although LPC NPs accumulated 6-fold higher in the spleen than in cisplatin-treated mice as shown in FIG. 22a, the data in FIG. 31 indicated that LPC NPs did not induce significant apoptosis spleen cells, which is consistent with other formulations.^{115, 116} It is believed that uptake is performed primarily by macrophages which can successfully internalize the NP to prevent cell apoptosis (FIG. 31). Therefore, the repair of CDDP-DNA adduct is also observed in spleen. However, other possible toxicities, such as hemosiderin deposition in spleen are not studied.¹¹⁷

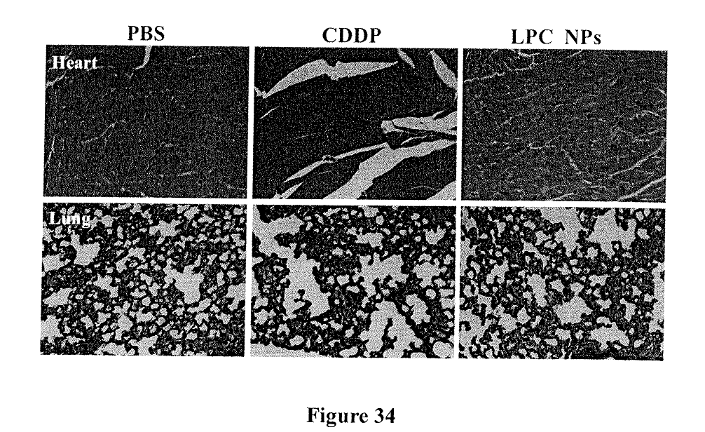

In clinics, the use of CDDP is mainly limited by nephrotoxicity. To this end, the nephrotoxicity of free CDDP and LPC NPs was studied. It was observed that LPC NPs induce significantly less nephrotoxicity over free CDDP at the same dose. As shown in FIG. 28, the morphology of kidneys treated with LPC NPs is similar to that treated with PBS. Therefore, no signs of nephrotoxicity are observed in kidneys from mice treated with LPC NPs while some nephrotoxicity is observed in mice treated with free CDDP. Glomeruloscelorsis, tubular cell atrophy, and cystic dilatation of renal tubes are observed in cells treated with free CDDP and indicated by rings, arrows, and squares, respectively. CDDP also induce significantly more apoptotic cells in kidney than LPC NPs (FIG. 31). In addition, there are no toxicities in heart and lung for both CDDP and LPC NPs. Pathologic examination of other major organs (lung and heart) in mice that received long-term treatments (FIG. 34) indicate that mice treated with LPC NPs suffered no organ damage.

These results indicate that while the neighboring effect is capable of inducing high levels of apoptosis in cancerous cells, its effects on healthy cells are nearly unobservable. A similar pattern is also observed in heart and lung cells in mice treated with LPC NPs. A key mechanism behind this observation is the formation of Pt-DNA adducts in both cancerous and healthy cells alike. However, the Pt-DNA adducts could be successfully repaired in healthy cells while they induced observable apoptosis in cancerous cells. The specificity of these NPs therefore allows a significant anti-tumor effect to achieve at a low dose of 1 mg/kg of Pt once a week for four weeks.

The antitumor efficacy of LPC NPs was tested in vitro and in vivo. When administered into mice at a low weekly dose, LPC NPs effectively inhibit the growth of melanoma tumors while free CDDP prove ineffective at the same dose and dosing schedule. In addition, LPC NPs also exhibit the neighboring effect both in vivo and in vitro. The successful uptake of LPC NPs by the tumor cells and the release of active drug following apoptosis further the effectiveness of the encapsulated drug. However, the neighboring effect is not induced in organ tissues due to their strong repair ability of the CDDP-DNA adduct. Thus, the tumor specific effect allows a magnification of anti-tumor efficacy at a low dose without pronounced side effects. As a consequence, both the therapeutic potential of CDDP and its safety toward normal tissues in vivo can be greatly optimized. As such, these studies show that the Pt drug delivery platform is an efficient and relatively safe candidate in the treatment of human melanoma tumors and a promising method for further explorations.

V. Lipsome-Encapsulated, Pure Cisplatin Nanoparticles with Tunable Size and Surface Modification for Cancer Therapy

The poor solubility of cisplatin (CDDP) often presents a major obstacle in the formulation of CDDP in nanoparticles (NPs) by traditional methods. A novel method is described herein for synthesizing CDDP NPs advantageously utilizing its poor solubility. By mixing two reverse microemulsions containing KCl and a highly soluble precursor of CDDP, cis-diaminedihydroplatinum (II), CDDP NPs have been successfully formulated with a controllable size (in the range of 12-75 nm) and high drug loading capacity (approximately 80 wt %).

The formulation is done in two steps. The pure CDDP NPs were first stabilized for dispersion in an organic solvent by coating with 1,2-dioleoyl-sn-glycero-3-phosphate (DOPA). Both x-ray photoelectron spectroscopy and ¹H NMR data confirmed that the major ingredient of the DOPA-coated NPs is CDDP. After purification, additional lipids were added to stabilize the NPs for dispersion in an aqueous solution. The final NPs contain a lipid bilayer coating and are named Lipid-Pt—Cl (LPC) NPs, which showed significant antitumor activity both in vitro and in vivo. This advantageous method of nanoparticle synthesis may also be applicable to the formulation of other insoluble drugs.

As noted above, in clinics, the maximum tolerated dose (MTD) of CDDP is significantly limited by nephrotoxicity.^{121, 122} To improve patient care, carboplatin and oxaliplatin are administered, while altering the chloride leaving groups of CDDP with 1,2-diaminocyclohexane or an oxalate ligand compromises outcome.¹²¹

In order to maintain the efficacy and reduce the nephrotoxicity of cisplatin, nanoparticulate CDDP formulations are very promising. Nanoparticulate CDDP formulations have been achieved through chelating CDDP with polymers and NPs,¹²³⁻¹²⁵ loading of a pro-drug in the PLGA NPs or encapsulating CDDP into liposomes.^{123, 126-133} For example, CDDP is loaded into PLGA NPs by exploiting double emulsion technique, while the encapsulation and loading efficacy is low and burst release is often observed.¹³⁴ Dhar et al utilized a prodrug strategy, i.e., modified the hydrophobicity of CDDP, and therefore improved the encapsulation of CDDP into PLGA NPs.^{133, 135, 136} Kataoka et al alternatively chelated CDDP positively charged platinum species to carboxylate-rich copolymers with a drug loading of 30wt % and showed a strong relationship between the therapeutic efficacy and the size of carrier.^{123, 124} Lipoplatin, a liposomal formulation, employed electrostatic interaction to load positively charged platinum into negatively charged DPPG-lipid micelles.^{137, 138} For Lipoplatin, reverse micelles were mixed with premade liposomes and homogenized by extrusion. Drug loading of Lipoplatin was reported to be 8.9 wt %. However, these formulations were for either prodrug or charged platinum, but not for native CDDP.

While the synthesis of CDDP (Scheme 2) is a well-documented reaction in the field of inorganic chemistry,¹³⁹ the poor solubility of CDDP in both water and organic solvents significantly hinders the development of nanoparticulate formulations in a manner similar to the formulation of nanoparticles with hydrophobic drugs.^{140, 141} Recently, a Lipid coated Calcium Phosphate (LCP) platform has been developed to deliver diverse bioactive molecules, such as DNA, silencing RNA and gemcitabine triphosphate.¹⁴²⁻¹⁴⁴ An outer layer of a cationic lipid (DOTAP) and high density of PEG was coated on the calcium phosphate cores. The cationic lipid DOTAP allows the nanoparticles to be internalized by tumor cells more efficiently and to subsequently escape from the lysosomes. Additionally, a high density of PEGylation will help the nanoparticles avoid RES system, improving drug pharmacokinetics and drug bioavailability. It was found that both components are critical for the successful delivery of drugs into tumors.

Calcium phosphate can be replaced by CDDP as the core in order to make CDDP nanoparticulate formulations. These formulations would be favorable due to its high drug loading capacity, e.g., as described elsewhere herein, at least about 10%, including about 80%. In another aspect, this platform is applicable to the manufacture of many other CDDP analog nanoparticulate formulations. This platform can improve the solubility of platinum based drug candidates with poor solubility, such as cis-diamminedibromoplatinum(II) and cis-diamminediiodoplatinum(II). As such, it is hypothesized that: (1) CDDP can be encapsulated as a nanoprecipitate in a microemulsion and stabilized in an organic solvent with 1, 2-dioleoyl-sn-glycero-3-phosphate (DOPA); (2) DOPA-coated CDDP NPs can be further dispersed into aqueous solution by adding lipids to form the outer leaflet of the coating bilayer; (3) the lipid bilayer-coated CDDP NPs will show anti-cancer activity in vitro and in vivo.

a. Synthesis and Characterization of DOPA-Coated CDDP Cores

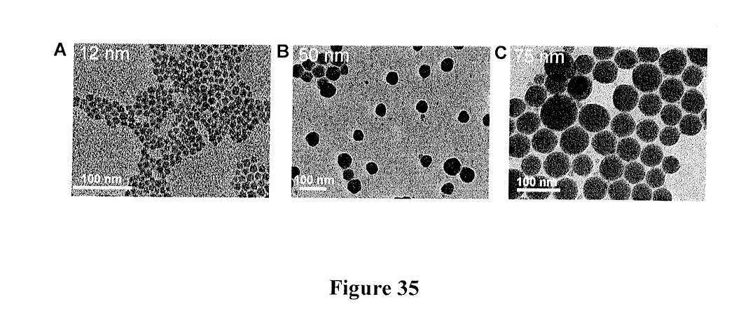

CDDP NPs were synthesized in microemulsion during the reaction between KCl and its highly soluble cis-[Pt(NH₃)₂(H₂O)₂](NO₃)₂ precursor. To synthesize stable CDDP precipitates, DOPA, which is known to strongly interact with the platinum cation at the interface,¹⁴⁵⁻¹⁴⁷ was used. To maximize the yield of CDDP NPs, an excess of KCl was used to inhibit hydrolysis equilibrium. After CDDP was precipitated, CDDP cores were coated with a hydrophobic layer of DOPA (Scheme 1). DOPA-coated CDDP NPs were purified in a manner similar to that of silica NPs, which were also synthesized in microemulsion. Ethanol was added to destroy the emulsion and precipitate CDDP NPs, which were collected by centrifugation. DOPA-coated CDDP NPs were readily dispersed in chloroform, toluene or hexane. By adjusting the composition of the surfactant system, the size of the NPs can be altered between 12 to 75 nm in diameter (FIG. 35). In addition, Lipid/Pt/Bromide (LPB) and Lipid/Pt/Iodide (LPI) can be formulated similarly, which contain cis-diaminedibromoplatinum(II) and cis-diaminediiodoplatinum(II), respectively.

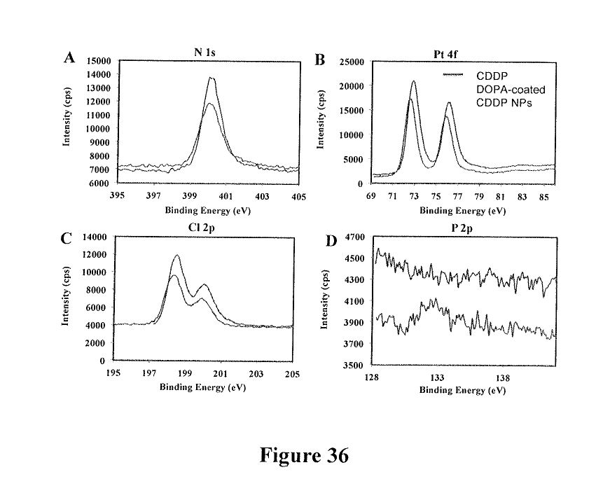

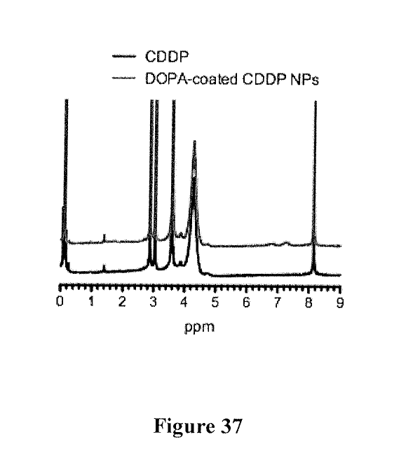

X-ray photoelectron spectroscopy (XPS) was used to confirm the composition of DOPA-coated CDDP NPs (FIG. 36). The ratio of N:Pt:Cl was 2:1:1.8, and a small amount of phosphorous element is also present from the presence of DOPA (FIG. 36D). In addition, no potassium element is found. The composition of DOPA-coated CDDP NPs was confirmed using ¹H NMR spectra in DMF-d7 (shown in FIG. 37). The data illustrated that the major peaks for DOPA-coated NPs are consistent with those of CDDP. Accordingly, NPs are composed of a CDDP core and coated with a layer of DOPA. The DOPA-coated CDDP NPs can have a substantially greater drug loading capacity. ICP-MS is used to measure the content of Pt and used NBD-labeled DOPA to measure the amount of DOPA on the surface of CDDP NPs. The results indicated that the yield of DOPA-coated CDDP NPs was approximately 45 wt %. The NPs were also characterized with a drug loading capacity as high as 93 wt %.

b. Facile Surface Engineering of Hydrophobic DOPA-Coated CDDP NPs with Outer Leaflet Lipids

To further disperse the hydrophobic DOPA-coated CDDP NPs in aqueous solution, additional lipids composed of 1,2-dioleoyl-3-trimethylammonium-propane (chloride salt) (DOTAP), cholesterol, DSPE-mPEG and DSPE-PEG-anisamide (molar ratio 4:4:1:1) were used. These lipids self-assembled in water into the outer leaflet of the bilayer through a hydrophobic interaction using DOPA-coated CDDP NPs as a template.^{148, 149} The DOPA layer served as the inner leaflet of the asymmetrical bilayer coating the CDDP core. The composition of the outer leaflet lipids was carefully chosen to contain a lipid (DSPE-mPEG) for prolonged circulation of NPs in the blood stream,¹⁵⁰ DSPE-PEG-anisamide for vivid uptake of NPs by the tumor cells and DOTAP for rupturing endosomes. ^{150, 151}

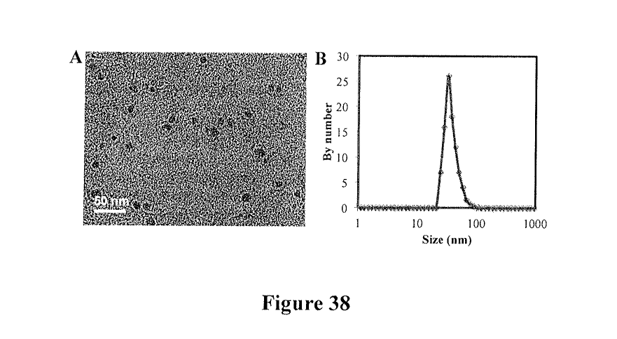

The final NPs are named Lipid-Pt—Cl (LPC) NPs. LPC NPs were purified via centrifugation to remove free liposomes formed as the result of excess outer leaflet lipids. LPC NPs were negatively stained with uranyl acetate and examined using TEM (FIG. 38A). The clear core/membrane structure revealed the bilayer coating the surface of the LPC NPs. As shown in FIG. 38A, the size of the particles determined using TEM is approximately 15 nm, which is smaller than the results obtained from DLS (FIG. 38B). The zeta potential of the NPs is +15 mV and DLS results showed that the distribution of LPC NPs is narrow, with a PDI of 0.15. The drug loading of LPC NPs is approximately 82 wt %. Overall, these results indicate LPC NPs are well dispersed in aqueous solution and characterized by a high drug loading.

c. In Vitro and In Vivo Anticancer Efficacy

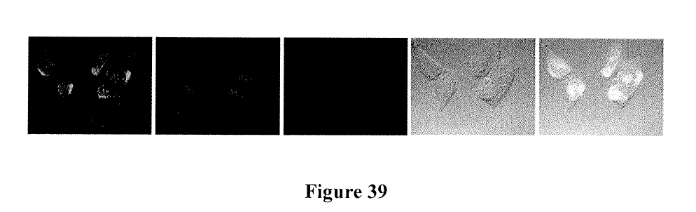

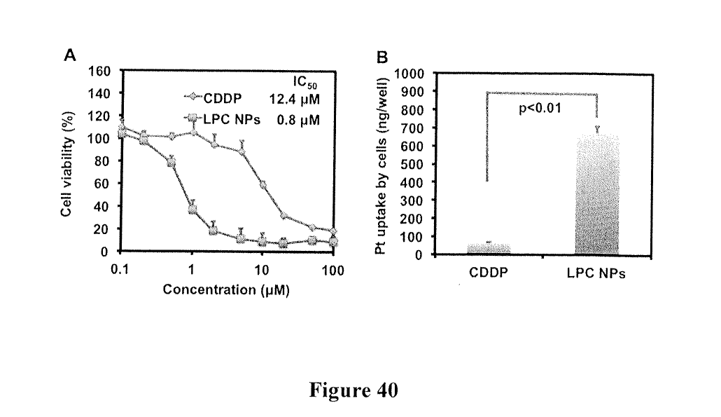

To test the anticancer efficacy of LPC NPs, the performance of LPC NPs was evaluated in 1205Lu melanoma cancer cells. DOPA-coated CDDP NPs with the size of 12 nm were used to prepare LPC NPs for the evaluation of anti-cancer effect. As shown in FIG. 40A, the IC₅₀ of CDDP and LPC NPs in 1205Lu cells were 12.4 and 0.80 μM respectively. Additionally, the empty liposome vesicles having a composition similar to that of the coating bilayer of LPC do not show any cytotoxicity (data not shown). The cellular uptake of LPC NPs was studied using confocal microscopy and ICP-MS. The LPC NPs were labeled using fluorescent NBD-PE lipid and incubated them with cells. Some of the LPC NPs were co-localized with lysosomes as shown by the yellow areas (FIG. 39). However, while a large number of NPs are endocytosed, they showed little accumulation in the lysosomes; a phenomenon that is facilitated by the capability of DOTAP to enhance endosome escape. Quantitative data indicated that cellular uptake of LCP NPs is more efficient than free CDDP (FIG. 40B). LPC NPs delivering CDDP showed an 11-fold increase in drug internalization over free CDDP, which explained the low IC₅₀ of LPC NPs.

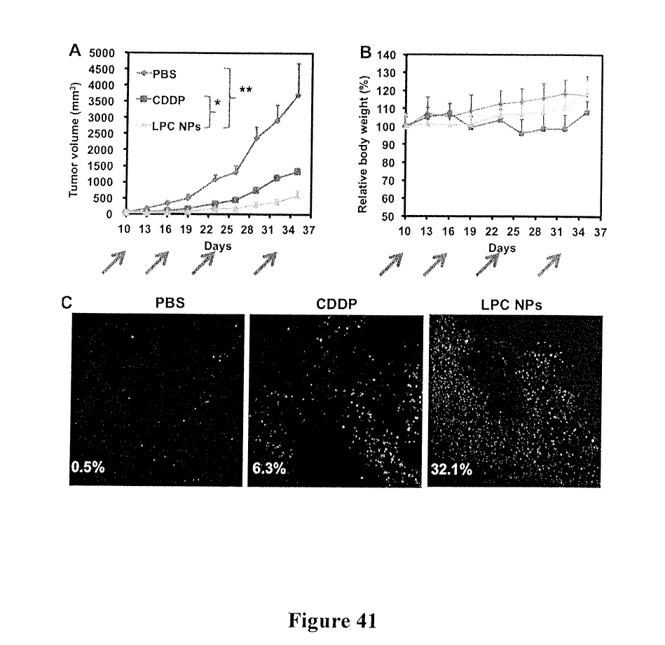

The efficacy of LPC NPs in a xenograft tumor model was further evaluated. FIG. 41A illustrates that free CDDP only has a partial effect on tumor growth inhibition at the dose and dose schedule used in the experiment. In contrast, tumor growth is significantly suppressed when LPC NPs are administered intravenously. In addition, mice in the treatment groups do not exhibit significant weight loss (FIG. 41B). Terminal deoxynucleotidyl transferase dUTP nick end labeling (TUNEL) assay is a method for detecting DNA fragmentation occurring in apoptosis by labeling the terminal end of nucleic acids. TUNEL images (FIG. 41C) indicate that LPC NPs induce noticeably more apoptosis (32.1%) than free CDDP (6.3%) do, which is consistent with the observed efficacy of tumor inhibition. Combined, this data suggest that the LPC NPs are both effective and safe in treating 1205Lu melanoma tumors.

Fabricated CDDP NPs were characterized by high drug loading capacity and optimal aqueous dispensability. Engineered via the microemulsion method and coated with DOPA and additional outer leaflet layers, LPC NPs exhibit significant antitumor effects both in vitro and in vivo. By adjusting the fabrication parameters, the size of the CDDP NPs can also be altered between 12-75 nm for optimal tumor accumulation. The synthesis of LPC NPs may be applicable to the formulation of other insoluble drugs. Notably, the cisplatin-based nanoparticle is prepared with the hydrophobic surface in organic solvent, not only allowing versatile coating and surface modification for a variety of purposes, in a manner similar to quantum dots and iron nanoparticles, but also allowing its co-encapsulation in amphiphilic polymers with other hydrophobic anti-cancer drugs. This cisplatin delivery system can be adapted for other similar drugs with low solubility.

V. Pharmaceutical Compositions and Methods of Delivery and Treatment

The delivery system complexes described herein are useful in mammalian tissue culture systems, in animal studies, and for therapeutic purposes. The delivery system complexes comprising a bioactive compound having therapeutic activity when expressed or introduced into a cell can be used in therapeutic applications. The delivery system complexes can be administered for therapeutic purposes or pharmaceutical compositions comprising the delivery system complexes along with additional pharmaceutical carriers can be formulated for delivery, i.e., administering to the subject, by any available route including, but not limited, to parenteral (e.g., intravenous), intradermal, subcutaneous, oral, nasal, bronchial, opthalmic, transdermal (topical), transmucosal, rectal, and vaginal routes. In some embodiments, the route of delivery is intravenous, parenteral, transmucosal, nasal, bronchial, vaginal, and oral.

As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds also can be incorporated into the compositions.

As one of ordinary skill in the art would appreciate, a presently disclosed pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions or dispersions such as those described elsewhere herein and sterile powders for the extemporaneous preparation of sterile injectable solutions or dispersions. For intravenous administration, suitable carriers include physiological saline, bacteriostatic water, or phosphate buffered saline (PBS). The composition should be sterile and should be fluid to the extent that easy syringability exists. In some embodiments, the pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols, such as manitol or sorbitol, or sodium chloride are included in the formulation. Prolonged absorption of the injectable formulation can be brought about by including in the formulation an agent that delays absorption, for example, aluminum monostearate and gelatin.

Sterile injectable solutions can be prepared by filter sterilization as described elsewhere herein. In certain embodiments, solutions for injection are free of endotoxin. Generally, dispersions are prepared by incorporating the delivery system complexes into a sterile vehicle which contains a basic dispersion medium and the required other ingredients from those enumerated above. In those embodiments in which sterile powders are used for the preparation of sterile injectable solutions, the solutions can be prepared by vacuum drying and freeze-drying which yields a powder of the active ingredient plus any additional desired ingredient from a previously sterile-filtered solution thereof.

Oral compositions generally include an inert diluent or an edible carrier. Oral compositions can be prepared using a fluid carrier for use as a mouthwash. Pharmaceutically compatible binding agents, and/or adjuvant materials can be included as part of the composition. The oral compositions can include a sweetening agent, such as sucrose or saccharin; or a flavoring agent, such as peppermint, methyl salicylate, or orange flavoring.

For administration by inhalation, the presently disclosed compositions can be delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical or cosmetic carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals. Guidance regarding dosing is provided elsewhere herein.

The present invention also includes an article of manufacture providing a delivery system complex described herein. The article of manufacture can include a vial or other container that contains a composition suitable for the present method together with any carrier, either dried or in liquid form. The article of manufacture further includes instructions in the form of a label on the container and/or in the form of an insert included in a box in which the container is packaged, for carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

The present invention provides methods for delivering a bioactive compound to a cell and for treating a disease or unwanted condition in a subject with a delivery system complex comprising a bioactive compound that has therapeutic activity against the disease or unwanted condition. Further provided herein are methods for making the presently disclosed delivery system complexes.

The presently disclosed delivery system complexes can be used to deliver the bioactive compound to cells by contacting a cell with the delivery system complexes. As described elsewhere herein, the term “deliver” when referring to a bioactive compound refers to the process resulting in the placement of the composition within the intracellular space of the cell or the extracellular space surrounding the cell. The term “cell” encompasses cells that are in culture and cells within a subject. The delivery of a polynucleotide into an intracellular space is also referred to as “transfection.” In these embodiments, the cells are contacted with the delivery system complex in such a manner as to allow the bioactive compounds comprised within the delivery system complexes to gain access to the interior of the cell.

The delivery of a bioactive compound to a cell can comprise an in vitro approach, an ex vivo approach, in which the delivery of the bioactive compound into a cell occurs outside of a subject (the transfected cells can then be transplanted into the subject), and an in vivo approach, wherein the delivery occurs within the subject itself.

In some embodiments, the exterior of the delivery system complex comprises a lipid-PEG conjugate. In some of these embodiments, the delivery system complex comprises a stealth delivery system complex. In certain embodiments, the outer leaflet of the liposome of the delivery system comprises a targeting ligand, thereby forming a targeted delivery system complex, wherein the targeting ligand targets the targeted delivery system complex to a targeted cell.

The delivery system complexes described herein comprising a bioactive compound can be used for the treatment of a disease or unwanted condition in a subject, wherein the bioactive compound has therapeutic activity against the disease or unwanted condition when expressed or introduced into a cell. The bioactive compound is administered to the subject in a therapeutically effective amount. In those embodiments wherein the bioactive compound comprises a polynucleotide, when the polynucleotide of interest is administered to a subject in therapeutically effective amounts, the polynucleotide of interest or the polypeptide encoded thereby is capable of treating the disease or unwanted condition.

By “therapeutic activity” when referring to a bioactive compound is intended that the molecule is able to elicit a desired pharmacological or physiological effect when administered to a subject in need thereof.

As used herein, the terms “treatment” or “prevention” refer to obtaining a desired pharmacologic and/or physiologic effect. The effect may be prophylactic in terms of completely or partially preventing a particular infection or disease or sign or symptom thereof and/or may be therapeutic in terms of a partial or complete cure of an infection or disease and/or adverse effect attributable to the infection or the disease. Accordingly, the method “prevents” (i.e., delays or inhibits) and/or “reduces” (i.e., decreases, slows, or ameliorates) the detrimental effects of a disease or disorder in the subject receiving the compositions of the invention. The subject may be any animal, including a mammal, such as a human, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The disease or unwanted condition to be treated can encompass any type of condition or disease that can be treated therapeutically. In some embodiments, the disease or unwanted condition that is to be treated is a cancer. As described elsewhere herein, the term “cancer” encompasses any type of unregulated cellular growth and includes all forms of cancer. In some embodiments, the cancer to be treated is a metastatic cancer. In particular, the cancer may be resistant to known therapies. Methods to detect the inhibition of cancer growth or progression are known in the art and include, but are not limited to, measuring the size of the primary tumor to detect a reduction in its size, delayed appearance of secondary tumors, slowed development of secondary tumors, decreased occurrence of secondary tumors, and slowed or decreased severity of secondary effects of disease.

It will be understood by one of skill in the art that the delivery system complexes can be used alone or in conjunction with other therapeutic modalities, including, but not limited to, surgical therapy, radiotherapy, or treatment with any type of therapeutic agent, such as a drug. In those embodiments in which the subject is afflicted with cancer, the delivery system complexes can be delivered in combination with any chemotherapeutic agent well known in the art.

When administered to a subject in need thereof, the delivery system complexes can further comprise a targeting ligand, as discussed elsewhere herein. In these embodiments, the targeting ligand will target the physically associated complex to a targeted cell or tissue within the subject. In certain embodiments, the targeted cell or tissue comprises a diseased cell or tissue or a cell or tissue characterized by the unwanted condition. In some of these embodiments, the delivery system complex is a stealth delivery system complex wherein the surface charge is shielded through the association of PEG molecules and the liposome further comprises a targeting ligand to direct the delivery system complex to targeted cells.

In some embodiments, particularly those in which the diameter of the delivery system complex is less than 100 nm, the delivery system complexes can be used to deliver bioactive compounds across the blood-brain barrier (BBB) into the central nervous system or across the placental barrier. Non-limiting examples of targeting ligands that can be used to target the BBB include transferring and lactoferrin (Huang et al. (2008) Biomaterials 29(2):238-246, which is herein incorporated by reference in its entirety). Further, the delivery system complexes can be transcytosed across the endothelium into both skeletal and cardiac muscle cells. For example, exon-skipping oligonucleotides can be delivered to treat Duchene muscular dystrophy (Moulton et al. (2009) Ann NY Acad Sci 1175:55-60, which is herein incorporated by reference in its entirety).

Delivery of a therapeutically effective amount of a delivery system complex comprising a bioactive compound can be obtained via administration of a pharmaceutical composition comprising a therapeutically effective dose of the bioactive compound or the delivery system complex. By “therapeutically effective amount” or “dose” is meant the concentration of a delivery system or a bioactive compound comprised therein that is sufficient to elicit the desired therapeutic effect.

As used herein, “effective amount” is an amount sufficient to effect beneficial or desired clinical or biochemical results. An effective amount can be administered one or more times.

The effective amount of the delivery system complex or bioactive compound will vary according to the weight, sex, age, and medical history of the subject. Other factors which influence the effective amount can include, but are not limited to, the severity of the subject's condition, the disorder being treated, the stability of the compound or complex, and, if desired, the adjuvant therapeutic agent being administered along with the polynucleotide delivery system. Methods to determine efficacy and dosage are known to those skilled in the art. See, for example, Isselbacher et al. (1996) Harrison's Principles of Internal Medicine 13 ed., 1814-1882, herein incorporated by reference.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic (e.g., immunotoxic) and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the presently disclosed methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical formulation can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, and the like. The skilled artisan will appreciate that certain factors can influence the dosage and timing required to effectively treat a subject, including but not limited to the severity of the disease, disorder, or unwanted condition, previous treatments, the general health and/or age of the subject, and other diseases or unwanted conditions present. Generally, treatment of a subject can include a single treatment or, in many cases, can include a series of treatments. Further, treatment of a subject can include a single cosmetic application or, in some embodiments, can include a series of cosmetic applications.

It is understood that appropriate doses of a compound depend upon its potency and can optionally be tailored to the particular recipient, for example, through administration of increasing doses until a preselected desired response is achieved. It is understood that the specific dose level for any particular animal subject can depend on a variety of factors including the activity of the specific compound employed, the age, body weight, general health, gender, and diet of the subject, the time of administration, the route of administration, the rate of excretion, any drug combination, and the degree of expression or activity to be modulated.

One of ordinary skill in the art upon review of the presently disclosed subject matter would appreciate that the presently disclosed compounds and pharmaceutical compositions thereof, can be administered directly to a cell, a cell culture, a cell culture medium, a tissue, a tissue culture, a tissue culture medium, and the like. When referring to the delivery systems of the invention, the term “administering,” and derivations thereof, comprises any method that allows for the compound to contact a cell. The presently disclosed compounds or pharmaceutical compositions thereof, can be administered to (or contacted with) a cell or a tissue in vitro or ex vivo. The presently disclosed compounds or pharmaceutical compositions thereof, also can be administered to (or contacted with) a cell or a tissue in vivo by administration to an individual subject, e.g., a patient, for example, by systemic administration (e.g., intravenous, intraperitoneal, intramuscular, subdermal, or intracranial administration) or topical application, as described elsewhere herein.

It is to be noted that the term “a” or “an” entity refers to one or more of that entity; for example, “a nanoparticle” is understood to represent one or more nanoparticles. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

The following examples are offered by way of illustration and not by way of limitation.

### Materials and Methods

### Materials:

Etoposide phosphate was purchased from Carbosynth (UK), 2-Dioleoyl-3-trimethylammonium-propanechloride salt (DOTAP), dioleoylphosphatydic acid (DOPA), and 1,2-distearoryl-snglycero-3-phosphoethanolamine-N- 8 methoxy(polyethyleneglycol-2000) ammonium salt (DSPE-PEG2000) were purchased from Avanti Polar Lipids, Inc. (Alabaster, Ala.) DSPE-PEG anisamide (AA) was synthesized according to the previously established procedure (80). All other chemicals were obtained from Sigma-Aldrich (St. Louis, Mo.) unless otherwise mentioned. All lipids were purchased from Avanti Polar Lipids (Alabaster, Ala.). DSPE-PEG-AA was synthesized in our lab.¹⁰ CDDP, AgNO₃ and other chemicals were obtained from Sigma-Aldrich (St Louis, Mo.) without further purification.

### Cell Culture:

H460 human NSCLC cells, obtained from American Type Culture Collection (ATCC), were cultured in an RPMI-1640 medium (Invitrogen, Carlsbad, Calif.) supplemented with 10% fetal bovine serum, 100 U/mL penicillin, and 100 mg/mL streptomycin (Invitrogen). Cells were cultivated in a humidified incubator at 37° C. and 5% CO₂.

### Experimental Animals:

Female nude mice and CD-1 mice of 6-8 weeks age were purchased from National Cancer Institute (Bethesda, Md.) and bred in the Division of Laboratory Animal Medicine (DLAM) at University of North Carolina at Chapel Hill. To establish the xenograft models, 5×10⁶ cells in 50 μL of PBS were injected subcutaneously into the right flank of the mice. All work performed on mice is approved by the Institutional Animal Care.

### Cell Lines

The human melanoma, A375M cell line was obtained from the American Type Culture Collection (ATCC, Manassas, Va.). A375M-GFP was constructed by transfecting an A375M cell line with pEGFP-N1 plasmid. The episomal expression of the plasmid in the transfected cells was maintained by cultivating the cells in the media containing Neomycin. All cells were cultured in DMEM medium supplemented with 10% heat-inactivated, fetal bovine serum (FBS), 20 mM of L-glutamine, 100 U/ml of penicillin G sodium, and 100 mg/ml of streptomycin at 37° C. in an atmosphere of 5% CO₂ and 95% air.

### EXAMPLES

### Example 1

### Preparation of Nanoparticles with High Loading of a Bioactive Compound that is an Insoluble Nano-Precipitate

NPs were prepared in micro-emulsions through a precipitation reaction between the highly soluble CDDP precursor and halide ions (such as chloride, bromide and iodide). (9,10). CDDP precursors and potassium halide salt were emulsified separately in two oil phase composed of Triton X-100, IGEPAL 520, and hexanol as co-surfactants in cyclohexane. To stabilize the final nanoparticle, dioleoylphosphatydic acid (DOPA) was added into the CDDP precursor's oil phase. After mixing the above two solutions, the core containing Pt nano-precipitate was washed three times by centrifugation using excess ethanol to remove cyclohexane and surfactants. The pellet was dissolved in CHCl₃ and stored in a glass vial for further modification. To prepare the LPC, the LPC core was mixed with DOTAP, cholesterol and DSPE-PEG2000 or DSPE-PEG2000-AA in CHCl₃. After evaporating the solvent, the residual lipid film was hydrated in d-H2O. LPB and LPI was prepared similarly. The yield, drug loading and encapsulation efficiency of drug was determined by measurement of P (lipid) and Pt (drug) content using ICP-MS.

The synthesis of cisplatin is a classic in inorganic chemistry. As shown in Scheme 2, cisplatin is nano-precipitated out of the reaction of KCl and the highly soluble cis-[Pt(NH₃)₂(H₂O)₂](NO₃)₂ precursor (54). This precipitation process was performed in a nano-reactor, i. e. in micro-emulsions. By using different halide ions (Cl, Br and I), LPC, LPB and LPI were prepared with sufficient yield (44 wt %) (Scheme 1).

TEM images showed that the drug cores were 15-30 nm in diamter. The lipid membrane was negatively stained with uranyl acetate and imaged to reveal the core/membrane nanostructure (FIG. 1). DLS results showed the hydrodynamic diameter of nanoparticles is slightly larger than the size indicated by TEM, in the range of 40 to 50 nm. To evaluate the kinetics of cisplatin release in the buffer, LPC and LPI were incubated at pH 7.4 at 37° C. over 144 h.

As shown in FIG. 3, LPC and LPI exhibited a sustained release of soluble Pt overtime. Of note, the rate of Pt release from LPI (t_{1/2}=80 h) was slower than that from LPC (t_{1/2}=45 h). This may be caused by difference in solubility between the two compounds. Compared to previous liposomal formulations, the delivery systems disclosed herein display controlled release properties without burst release. Additionally, the release rate can be adjusted by the use of different halide ions.

### Example 2

### In Vitro and In Vivo Activity

The performance of LPC and LPI was evaluated in cultured cells and in tumor models. In 1205Lu and A375M human melanoma cancer cell lines, LPC and LPI showed comparable cytotoxicity in vitro. IC₅₀ after 48 h was about 10 μM for both the LPC and LPI formulations in both cell lines. Although nanoparticles can efficiently transfer drugs into cells, the release of drug intracellularly is not instantaneous but sustainable. This is also confirmed by cell uptake experiment. As shown in FIG. 4, cells displayed normal morphology after incubated with LPC for 4 h. However, cells showed abnormal morphology after being exposed to CDDP for 4 h and became apoptotic (photo not shown). In addition, DOTAP helped the nanoparticles escape from lysosomes which were labeled with red. Most of the green NBD-PE labeled nanoparticles were located away from red lysosomes.

The small molecular CDDP is cleared quickly in vivo. However, nanoparticulate formulation can make the drug's in vivo retention much longer. After 4 h of I. V. injection, about 15% of the total injected dose was still in the circulation (FIG. 5). The distribution of the drug in the 1205Lu tumor in nude mice was as high as 20% injected dose per gram. The longer circulation time in the blood and higher accumulation in the tumor would favor effective inhibition of tumor growth even if the drug is not released rapidly. Further, the slow release might be beneficial for a less frequent dosing schedule.

The performance of an exemplary formulation in 1205Lu and A375M was tested in melanoma tumor xenograft models. In the 1205Lu tumor model, the drugs are administered by I. V. injection weekly at the dose of 2.0 mg/kg Pt. FIG. 2A shows that both LPC and LPI could inhibit the tumor growth significantly, without reducing body weight of the treated animals (FIG. 2B). These results indicate that the slow release property is good for longer injection intervals, which will reduce toxicity.

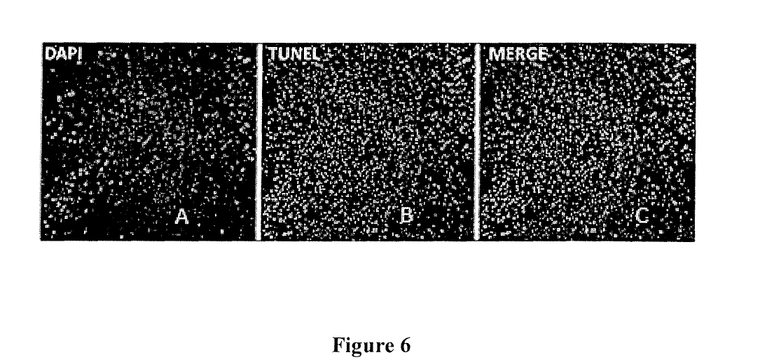

The efficacy of LPC's was also tested in A375M tumor. When the tumors were well established (mean volume 600 mm³), mice received 2 weekly injections of LPC at the dose of 3 mg/kg. The LPC exhibited remarkable antitumor activity in A375M tumors. The tumors did not grow but rather shrunk about 40% in volume. Seven days after the second injection, about 90% of the tumor cells were apoptotic as illustrated by a TUNEL assay (FIG. 6). Since it is unlikely that 90% of all cells in the tumor had been contacted by the NPs, extensive cell apoptosis may result from the drug released from those cells taken up the NPs. Such “innocent bystander” cell death might be a very important feature of the Pt NPs disclosed herein. Thus, these results indicate that LPC and LPI exhibited very effective and long lasting anti-tumor activity in human melanoma models.

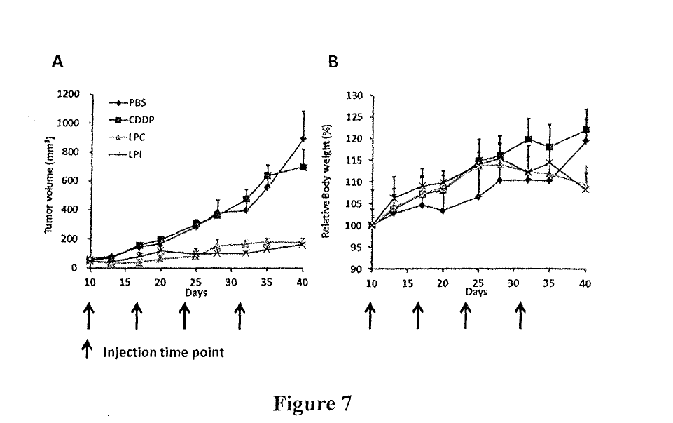

A low dose experiment with A375M tumor model was performed. As shown in FIG. 7, both LPC and LPI at the weekly dose of 1 mg/kg significantly inhibited tumor growth of A375M in a xenograft model. At the same dose and dosing schedule, free CDDP was not effective at all. Since many B-Raf^{V600E} melanoma patients develop resistant to Vemurafenib (Vem), Pt drug formulations described herein were tested for efficacy against this resistant-type of tumor.

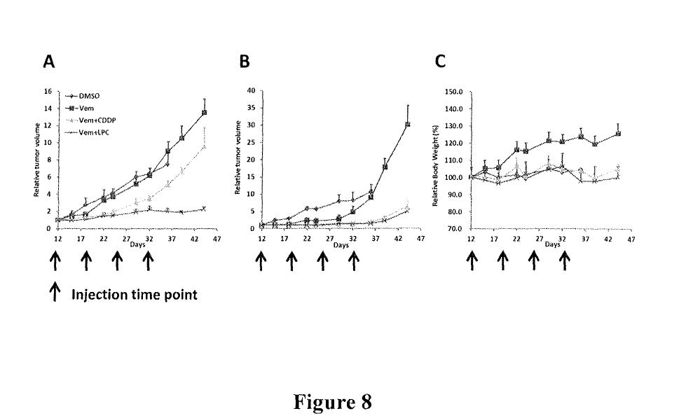

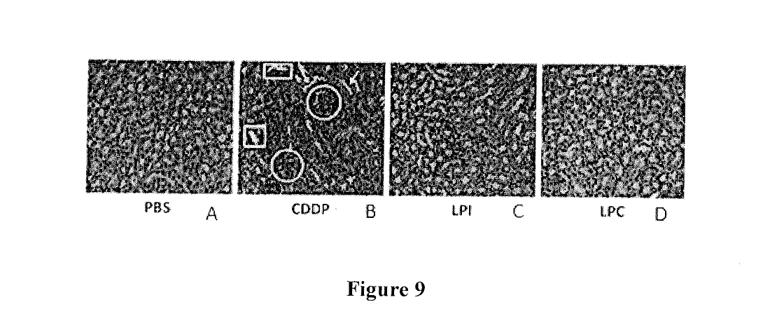

In the experiment shown in FIG. 8, Vem-resistant and Vem-sensitive 1205Lu tumors were tested for comparison. Each animal was inoculated with both resistant and sensitive tumors on the separate side of the body. After 12 daily Vem injections (100 mg/Kg), mice were randomly divided into 4 groups. Each group had 4-6 mice. And 2.0 mg/Kg of Pt drugs was administered by I.V injection for Vem+CDDP and Vem+LPC groups; 100 mg/Kg Vem was administered daily by I.P. injection to Vem, Vem+CDDP and Vem+LPC groups. As can be seen from the FIG. 8A, Vem-resistant tumor grew rapidly despite of the daily dose of Vem. But the tumor failed to grow when Vem and LPC were administered together. Free CDDP had a partial effect when administered together with Vem. However, as soon as the dosing of CDDP terminated, tumor resumed growth. Of note, the resistant tumor stayed without growth even 12 days after last LPC dose. Again, the result strongly suggested a sustained release effect for the LPC formulation. FIG. 9B shows the data for the sensitive tumor. The tumor became Vem-resistant only after 30 daily doses. Both free CDDP and LPC were effective in arresting the tumor growth.

To evaluate the kidney toxicity, organs were taken for histopathological observation using H&E staining. As can be seen from FIG. 9, nephrotoxicity signals such as glomeruloscelorosis (yellow rings), tubular cell atrophy (arrows) and cystic dilatation of the most renal tubules (squares) were found in the group treated with CDDP alone. No nephrotoxicity was observed in LPC and LPI treatment groups. We also did a pathologic examination by H&E staining of other major organs (liver, lung, spleen and heart) from mice that received long-term treatments (not shown). No organ damage was observed in mice treated with either LPC or LPI. Finally, we tested the blood parameters. Levels of secreted liver enzymes (AST and ALT), and blood urea nitrogen were all unchanged after a four-dose treatment with LPC or LPI, indicating a lack of damage to the liver and the kidneys (Table 2). Considered together, these results show the safety of LPC and LPI formulations.

TABLE 2
Kidney and liver function parameters.

| | AST(U/L) | ALT(U/L) | BUN(mg/dl) |
|---|---|---|---|
| PBS | 146.0 ± 42.1 | 51.7 ± 3.5 | 34.3 ± 3.1 |
| CDDP | 105.7 ± 27.1 | 54.3 ± 9.7 | 25.7 ± 0.6 |
| LPI | 135.7 ± 8.6 | 59.0 ± 6.1 | 28.7 ± 2.3 |
| LPC | 116.0 ± 7.6 | 53 ± 4.9 | 34 ± 2.8 |
| Normal Range | 54-298 | 17-77 | 8-33 |

Values are as mean ± SD;
AST, aspartate aminotransferase;
ALT, alanine aminotransferase;
BUN, blood urea nitrogen.

### Example 3

### Characterizing of Delivery Complexes

The zeta potential and particle size of LPX will be further determined by dynamic light scattering and negative-stain TEM. The yield, drug loading and encapsulation efficiency of drugs will be determined by measurement of P (lipid) and Pt (drug) content using ICP-MS. The crystal structure of the nano-precipitated drugs will be analyzed by TEM-EDS and XPS techniques. The drug release rate of LPX will be determined using a dialysis method at 37° C. in HEPES buffer (pH=7.4). Released drug concentration will be measured by ICP-MS. The effect of salt concentration and pH of buffer on release rate will be investigated.

### Example 4

### In vivo Assays

The efficacy of LPX will be further studied in cultured cells. After incubation with drugs, cells will be cultured for 48 h. Then, IC₅₀ of the free drug and the NP formulation will be evaluated by MTS assay. The distribution of LPX labeled with a fluorescence lipid will be observed using confocal microscopy. Lysosomes will be labeled with Lysotracker. Co-localization of LPX with lysosomes will be investigated. Endosomal escape of the NPs may depend on the presence of a cationic lipid in the outer leaflet of the wrapping bilayer. It is noted that LPX containing a neutral lipid, such as dioleoyl phosphatidylcholine (DOPC) may accumulate in the lysosomes and reduce the bioavailability of the drug.

To study pharmacokinetics, data will be collected at multiple time points (0, 15, 30, 45 and 60 min and 2, 4, 8, 24, 48 h) in order to obtain the entire clearance profile. At least 5 animals per time point will be included to assure statistical significance. The concentration of Pt will be assayed by ICP-MS, the sensitivity of which is very high. The biodistribution in different organs, including the tumors, will be similarly determined. The biological activity of LPX will be ascertained in the 1205Lu and A375M xenograft models. Lower effective doses will essentially eliminate the possibility of toxicity with CDDP delivery, at least in the mouse model. Mice are administered by I .V. injection weekly at the dose of 0.5 mg/kg, 1, 2 and 3 mg/kg. The inhibition efficacy will be demonstrated by the changes in tumor size, PCNA and TUNEL assays.

CDDP resistant tumor models will also be further tested. In addition to the CDDP resistant tumors, other drug resistant tumors will also be treated with the combination therapy. Specifically, Vemurafenib resistant melanoma will be tested for its sensitivity to LPX alone or in combination with Vemurafenib. In all experiments, free CDDP will be used as a positive control for comparison. Slower growing tumors, such as A375M, which is about 3-fold slower in growth in the nude mouse than 1205Lu are of particular interest. A375M may respond to LPI better than LPC in terms of the minimal effective dose and dosing schedule due to the slower drug release rate of the former. The dosing schedule will be varied from once a week to once 2 or 3 weeks. LPI might be particularly suitable for infrequent dosing, again due to its slow drug release rate.

For safety evaluation, maximum tolerable dose is measured. Systemic toxicity of LPX is examined by histological and biochemical analyses in normal mice (CD1). Female CD1 mice, 5 in each group, are injected with LPX at the dose of 2.0 mg/kg Pt weekly for one month and sacrificed 7 days after the last injection. Major organs, including the liver, heart, lung, kidney and spleen (for histological evaluation), and blood are collected. The clinical chemistry parameters in blood is determined, including glucose, BUN, creatinine, bilirubin, total protein, albumin, ALT, AST, alkaline phosphatase, Na+, K+, Ca+, chloride, and inorganic phosphorus.

Data disclosed elsewhere herein have already indicated strong response to LPC in two melanoma cell lines. LPI might be more active than LPC with lower dose and/or longer dosing intervals in slower growing tumors. Although A375M grows 3-fold slower than 1205Lu, it is not the slowest growing human tumor.

Pancreatic tumor lines will be employed to test the efficacy against pancreatic cancer. Several pancreatic tumor lines grow very slowly in nude mouse. Tumor growth requires about months to reach about 300 mm³. Although CDDP is not clinically active in pancreatic cancer, our nanoparticle formulation may show activity in these slow growers.

Pt drugs released from cells taken up LPX can kill the neighboring “innocent” cells. As shown in FIG. 5, cells took up a lot of fluorescence labeled LPC by 4 h and stayed healthy. These cells will be extensively washed, trypsinized and mixed with fresh untreated cells at different ratios and re-cultured in the absence of any additional drugs. Apoptosis of cells near the labeled cells will be analyzed at different time. To examine if the “innocent bystander” cell killing occurs in vivo, tumor bearing mice will be injected with LPX labeled with a red fluorescence lipid, such as rhodamine-PE, which will mark the tumor cells taken up the NPs. Tumor will be harvested at different times up to 2 days after the injection and fixed and embedded in paraffin. Thin sections will be stained for TUNEL for apoptotic cells (green) and scoring % of green cells that are not red. LPI may show a different kinetics in killing the innocent bystander cells than LPC.

### Example 5

### Preparation of Etoposide Phosphate Loaded Indium Nanoparticles (IEP)

The IEP core particles were prepared (81) with modifications. Briefly, two hundred and fifty uL of 20 mM etoposide phosphate solution is added to 20 mL oil phase containing cyclohexane/IgepalCO-520/triton-100/Hexanol solution (71.25/22.5/3.75/2.5, v/v) with continuous stifling and another micro emulsion was prepared by using 250 uL of 100 mM Indium chloride. 300 uL of DOPA (25 mg/mL) solution was added to the drug containing oil phase. After approximately five minutes, two separate micro-emulsions were mixed and stirred continuously for 20 min before the addition of 40 mL of absolute ethanol. The resultant solution was centrifuged at 10000×g for 20 min to pellet IEP core. The same procedure was repeated twice to remove the surfactant and the core was dried under nitrogen. Finally, the core particles were dissolved in chloroform and stored at −20° C. The final particles were made by using conventional liposomal preparation method, containing DOPC, Chol and DSPE PEG (1:1:0.1 mole ratios). The IEP core particles along with the lipids were in chloroform. The solvent was then removed under the reduced pressure to form a dry lipid film. The final particles were prepared by hydration and sonication. The AA-targeted IEP were prepared in the same way by replacing 10% DSPE-PEG with DSPE-PEG-AA.

a. Characterization of IEP Nanoparticles:

Transmission electron microscope (TEM) images of IEP core particles were acquired by JEOL 100CX II TEM (Tokyo, Japan). The Energy dispersive X-ray spectroscopy (EDS) results were obtained by JEOL 2010F FaSTEM, 200 kV accelerating voltage connected to Oxford X-mas system. The 300 mesh carbon coated copper grid (Ted Pella, Inc., Redding, Calif.) were used to prepare samples for TEM and EDS. The particle size and zeta potential of final lipid coated IEP nanoparticles were determined by dynamic light scattering (DLS) using Malvern ZetaSizer Nano series (Westborough, Mass.). Encapsulation efficiency of etoposide phosphate was measured by a UV/Vis spectrophotometer (Beckman Coulter Inc., DU 800). The mass spectrum was obtained by using LCMS (Shimadzu).

b. In Vitro Cellular Uptake:

Cellular uptake of nanoparticles byH460 cancer cells was observed by using dual labeled nanoparticles. The core particles were labeled with NBD-DOPA and outer leaflet was labeled with DiI. The labeled particles were incubated for 4 h, washed twice with PBS followed by fixing with 4% paraformaldehyde and nucleus staining with DAPI (Vector laboratories, Calif.). Fluorescent pictures were taken using Olympus FV1000 MPE SIM Laser Scanning Confocal Microscope (Olympus).

c. In Vitro Cell Ciability Assay (MTT):

In vitro cell viability of IEP nanoparticles was determined by using the 3-[4,5-dimethylthiazol-2-yl]-2,5-diphenyltetrazolium bromide (MTT) assay. H460 cells were seeded at a density of 1×10⁴ per well in96-well plates 24 hours prior to treatment. The cells were treated with different concentrations of IEP-PEG, IEP-PEG AA, free Indium chloride, free outer liposome and free etoposide phosphate for 36 h. The concentrations of indium chloride and free liposomes were maintained in equal mounts used for IEP-PEG and IEP-PEG AA. The medium was replaced with fresh medium containing 5% MTT (Biosynth Inc.) solution and incubated at 37° C. for another 4 h. The resulting formazan crystals were solubilized by adding 100 μL DMSO/Methanol (50:50) solution to each well. The absorbance at 570 nm wavelength was measured with a micro plate reader. Cell viability was calculated as the percentage of the absorbance of the treated cells to that of untreated cells.

d. Cell Cycle Analysis:

H460 cells (2×10⁵) were seeded in 6-well plates 24 h prior to the treatment. The formulations, IEP-PEG, IEP-PEG AA, InCl₃, free liposome and free drug were added and incubated for 24 h at 37° C., humidified CO₂ incubator. The cells were trypsinized and washed with PBS followed by fixation in pre-cooled 70% ethanol at −20° C. for at least 1 h. Fixed cells were washed with PBS staining buffer (BD Pharmingen, San Diego, Calif.) and incubated with RNAase (final concentration 75 mg/mL) at 37° C. for 30 min, followed by incubation with 10 mg propidium iodide (PI) at room temperature for 30 min. Finally, cells were washed and suspended in PBS, and analyzed with a FACS Canto flow cytometer (BD Biosciences) to measure the PI intensity, which correlates with the DNA content in the cell cycle. A total of 20,000 events are acquired for each sample and data were analyzed with FACS Diva software (BD Biosciences).

e. Caspase Activation Assay:

H460 cells (2×10⁵) were treated as mentioned above with all formulations. The cells were lysed with a radio-immunoprecipitation assay (RIPA) buffer that was supplemented with a protease inhibitor cocktail (Promega, Madison, Wis.). The protein lysates were collected by centrifugation at 14,000 rpm for 10 min at 4° C. Protein concentrations were determined using the BCA assay kit (Pierce Biotechnology) following the manufacturer's recommendations. Thirty micrograms protein of each sample was used to detect caspase-3/7 activity of the cell lysates by using an in vitro assay kit according to the manufacturer's instructions (Promega).

f. Western Blot Analysis for PARP:

Forty micrograms of protein per lane was resolved by 4%-12% SDS-PAG Electrophoresis (Invitrogen) before being transferred to polyvinylidenedifluoride (PVDF) membranes (Bio-Rad). The membranes were blocked for 1 h with 5% skim milk at room temperature and then incubated with mouse monoclonal poly (ADPribose) polymerase-1 (PARP-1) antibodies (1:500 dilution; Santa Cruz biotechnology, Inc.) and with β-actin antibody (1:4000 dilution; Santa Cruz biotechnology, Inc.) overnight at 4° C. β-actin was probed as the loading control. The membranes were washed 3 times and then incubated with a secondary antibody (1:4000 dilutions; Santa Cruz biotechnology, Inc.) at room temperature for 1 h. Goat anti-mouse secondary antibody is used for PARP and β-actin primary antibody. Finally, the membranes were washed 4 times and developed by an enhanced chemiluminescence system according to the manufacturer's instructions (Thermo scientific).

g. TUNEL and Immunohistochemistry Assay:

In vivo tumor cell apoptosis was determined by TdT-mediated dUTP Nick-End Labeling (TUNEL) assay. H460 tumor bearing mice were given three daily IV injections of IEP-PEG, IEP-PEG AA and free EP at dose of 5 mg/kg (n=3). Twenty-four hours after the final injection, mice were sacrificed and tumors are fixed in 4% paraformaldehyde solution for 12 h before being embedded in paraffin and sectioned at a thickness of 5 μm. The TUNEL staining was performed as recommended by the manufacturer (Promega). DAPI mounting medium (Vector Laboratories, Inc., Burlingame, Calif.) was dropped on the sections for nucleus staining. Images of TUNEL-stained tumor sections were captured with a fluorescence microscope (Nikon Corp., Tokyo, Japan). The percentage of apoptotic cells was obtained by dividing the number of apoptotic cells(TUNEL positive cells shown as green dots) from the number of total cells(blue nuclei stained by DAPI, not shown) in each microscopic field, and 10 representative microscopic fields were randomly selected in each treatment group for this analysis. Proliferation of tumor cells after the aforementioned treatments and dosing schedule was detected by immunohistochemistry, using an antibody against proliferating cell nuclear antigen (PCNA) (1:200 dilution, Santa Cruz). The immunohistochemistry was performed using a mouse-specific HRP/DAB detection IHC kit as recommended by the manufacturer (Abcam, Cambridge, Mass.). The percentage of proliferation cells was obtained by dividing the number of PCNA positive cells(shown as brown dots) from the number of total cells (blue nuclei stained by hematoxylin) in each microscopic field, and 10 representative microscopic fields are randomly selected in each treatment group (n=3) for counting.

h. Tumor Growth Inhibition and Toxicity Study:

A tumor growth inhibition study was completed on H460 subcutaneous xenograft mouse models. Mice were inoculated with 5×10⁶ H460cells by subcutaneous injection. Treatment was started when the tumor volumes reached about 100-150mm³. The mice were randomly assigned into treatment groups (n=5), and intravenously injected different formulations, including IEP-PEG, IEP-PEG AA and free EP. Four injections were performed every other day for a total of 4 injections at an EP dose of 5 mg/kg. Tumor sizes were measured every other day with calipers across their two perpendicular diameters, and the tumor volume was calculated using the following formula: V=0.5×(W×W'3L), where V is tumor volume, W is the smaller perpendicular diameter and L is the larger perpendicular diameter. Body weight of each mouse is recorded every other day. Humane sacrifice of mice was performed when tumors reached 20 mm in one dimension.

i. In Vivo Bio-Distribution:

In vivo bio distribution of IEP nanoparticles were measured by using radio labeled Indium (¹¹¹InCl₃, PerkinElmer, Inc.). H460 tumor bearing nude mice were treated with IEP-PEG and IEP-PEG AA particles intravenously (n=5). After 6 h, organs were collected followed by measuring of ¹¹¹In amount. The results were plotted percentage injected dose per gram tissue in different organs.

j. In Vivo Safety Studies:

CD-1 mice were treated with IEP-PEG and IEP-PEG AA particles intravenously every other day for three times. After one day mice were sacrificed and organs were collected and fixed in 4% paraformaldehyde solution followed by H&E staining. The pictures were taken by using fluorescent microscope (Nikon, Japan) under bright filed.

k. Statistical Analysis:

Results were expressed as a mean±standard deviation (SD) and were compared among different groups using Student's t-test. P<0.05 is considered as statistical significant.

### Characterization of IEP Nanoparticles:

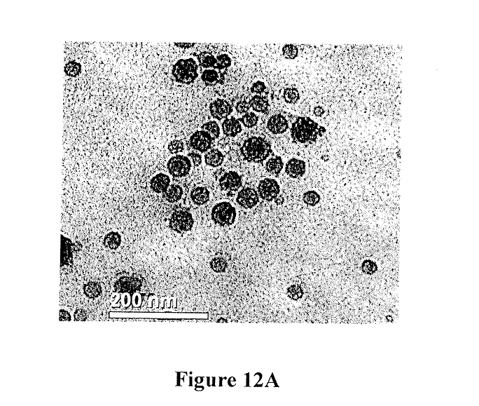

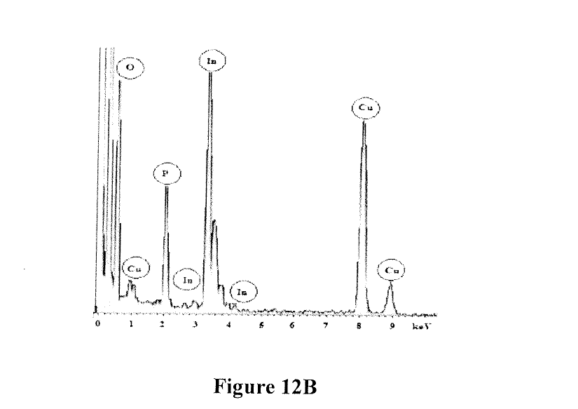

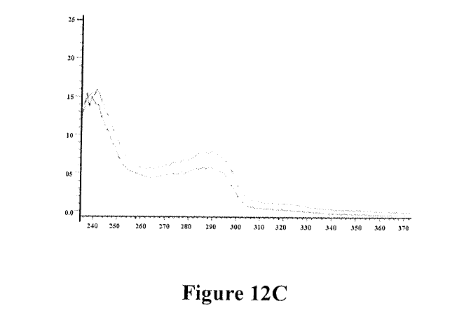

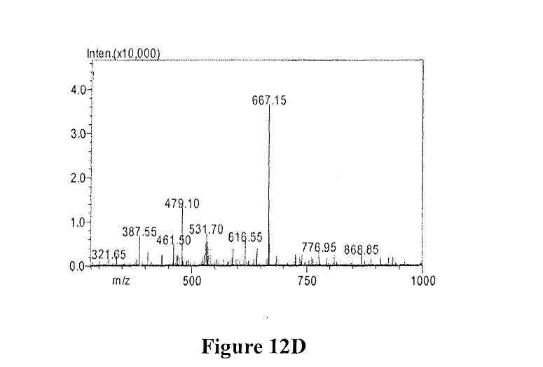

First, the IEP cores of nanoparticles were analyzed using high resolution TEM to evaluate morphology and size. The nanoparticles were spherical with a ˜45 nm diameter (FIG. 12A). Energy dispersive X-ray spectroscopy (EDS) confirmed the presence of indium and phosphate in the particles (FIG. 12B). The amount of etoposide phosphate (EP) encapsulated in the DOPA-stabilized IEP core was measured by UV-Vis spectroscopy (FIG. 12C), by using a standard curve for free drug EP. It was found that 60-65% of the drug is encapsulated in the nanoparticles.

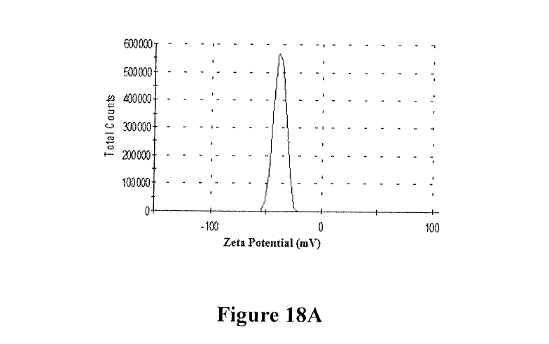

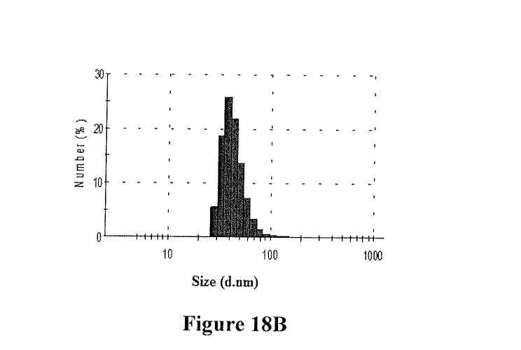

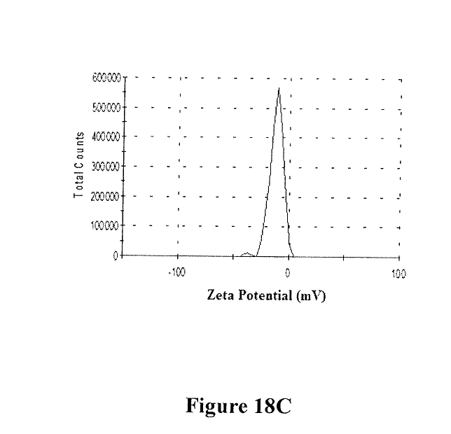

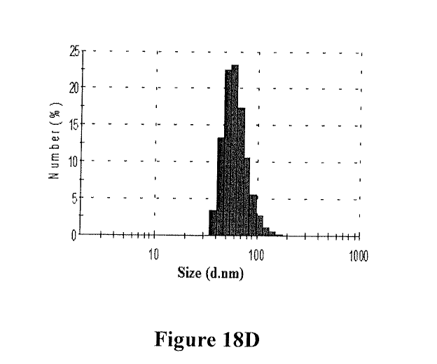

The EP structure was analyzed by ESI-MS and (FIG. 12D) showed that free and nanoparticle-associated EP has similar mass, confirming that the structure of the drug is not changed by the nanoparticle preparation process. Fluorescent particles made with NBD-DOPA confirmed that the IEP core particles were coated with DOPA lipid (data not shown). Final particles were PEGylated to lengthen time in circulation and enhance tumor accumulation. The outer leaflet was composed of DOPC:Chol:DSPE-PEG (1:1:0.1). To target sigma receptor over expressing cancer cells, 50% of the DSPE-PEG was replaced with DSPE-PEG-AA. The final particle size and zeta potential were measured by dynamic light scattering (DLS; FIG. 18) and Doppler laser velocimetry. The particle sizes of the non-targeted and targeted nanoparticles are similar, ˜55 nm. The zeta potential is −40 mV for IEP-PEG particles and is −10 mV for IEP-PEG AA.

### Receptor Mediated Cellular Uptake of IEP Nanoparticles:

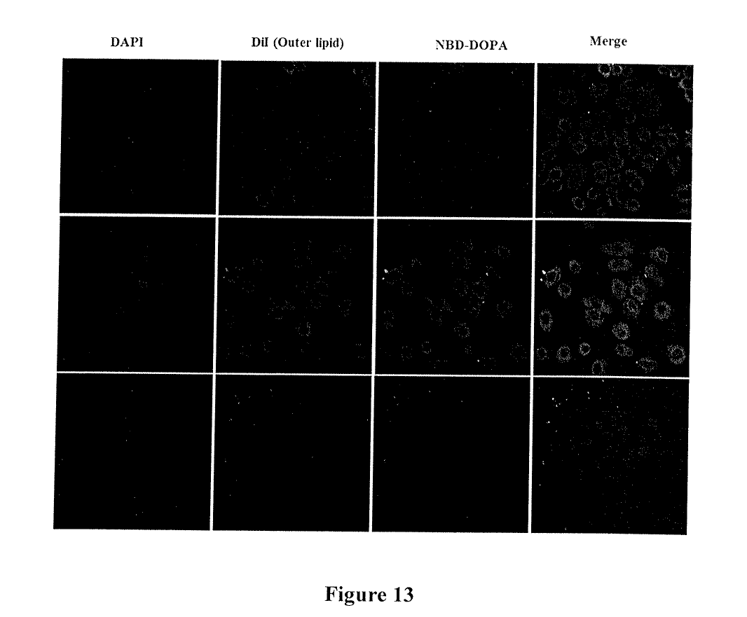

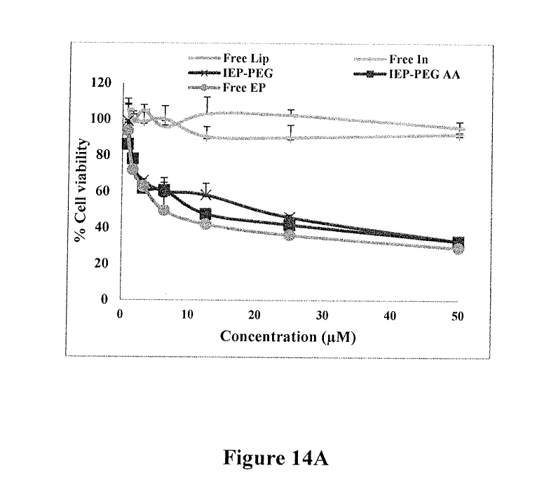

To confirm asymmetric membrane coating of the IEP core, the inner and outer leaflets of IEP nanoparticles were labeled with NBD and DiI respectively. Nanoparticles were then added to H460 cells in culture. Red and green fluorescence showed high co-localization in the cells, indirectly confirming distinct composition of lipid coatings. IEP nanoparticles efficiently entered tumor cells in culture (FIG. 13). Sigma receptors are often over expressed on tumor cells including the H460 tumor cell line (81). Increased cellular uptake is seen with the targeted nanoparticle (IEP-PEGAA) relative to untargeted (IEP-PEG) and the increase could be blocked by the sigma receptor agonist haloperidol as shown by a significant drop in fluorescence intensity. These results are consistent with receptor specific delivery of cargo to the cytoplasm of tumor cells by AA targeted IEP nanoparticles.

### In Vitro Anti-Cancer Activity of IEP Nanoparticles Determined by MTT Assay:

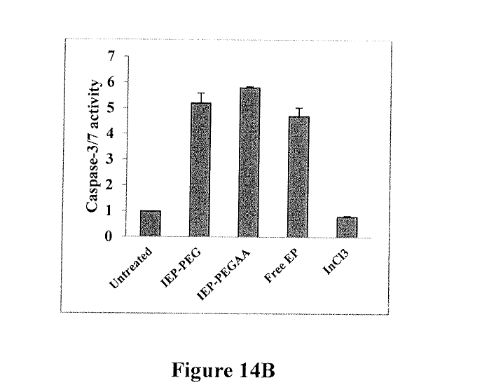

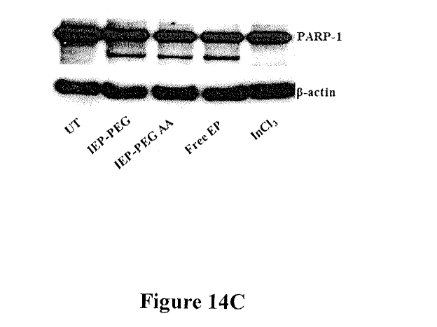

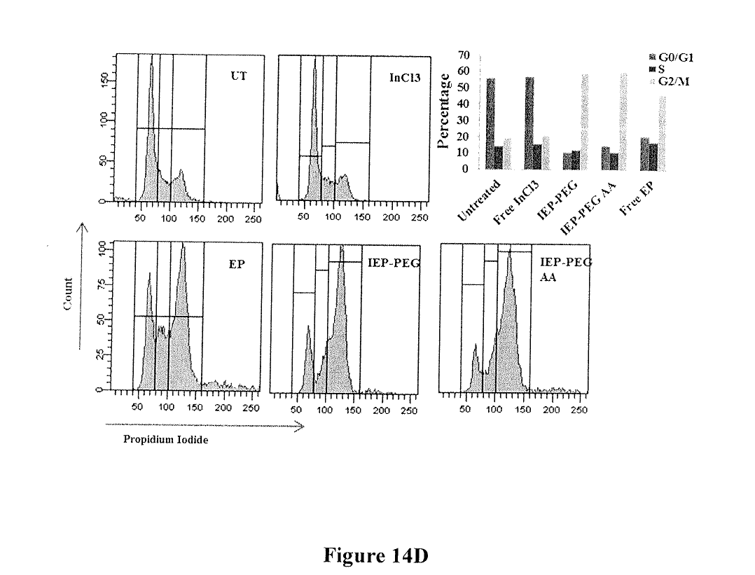

The in vitro anti-cancer activity of IEP nanoparticles was measured by MTT assay in cultured NCI-H460 lung carcinoma cell lines. Cells are treated with different formulations for 36 h. IEP nanoparticles and free drug exhibited a dose-dependent toxicity in lung cancer cells (FIG. 14A). Free indium chloride and free outer liposomes alone had no effect on viability in a manner consistent with the observed cytotoxicity being associated with EP and not indium or the lipid coating on the nanoparticles.

### IEP Nanoparticles Induced Apoptosis in Cultured NCI-H460 Cells by Caspase-Dependent Mechanism:

DNA damage induces apoptosis through the activity of caspase-type proteases (82), primarily caspases 3 and 7. Their activity was 5-6 fold higher in IEP nanoparticle treated and 4 fold higher in free EP treated NCI H-460 cells relative to untreated control (FIG. 14B). Indium chloride alone has no effect. Poly ADP ribose polymerase (PARP-1), an enzyme involved in DNA repair, is known to be cleaved by caspases into 24 kDa and 89 kDa fragments (83) during the execution of apoptosis. An increase in the 89 kDa cleavage product is readily detectable in cells treated with targeted and untargeted IEP nanoparticles or free etoposide phosphate (FIG. 14C) while free indium chloride again had no effect, consistent with the DNA damage-induced toxicity being due to EP.

### EP-Dependent Cell Cycle Analysis by Flow Cytometer:

The effect of IEP nanoparticles on the cell cycle was evaluated using flow cytometry. Etoposide inhibits topoisomerase II, resulting in DNA strand breakage which in turn causes cell cycle arrest at late S or early G2/M phase(84). Flow cytometry (FIG. 14D) demonstrated that about 60% of the cells are found at G2/M phase after treatment with IEP nanoparticles. About 50% of cells treated with free EP are arrested at G2/M phase, while free indium chloride had no discernable effect. These results are consistent with EP anticancer activity in our nanoparticle system.

### Systemic Toxicity and Bio-Distribution of Nanoparticles In Vivo:

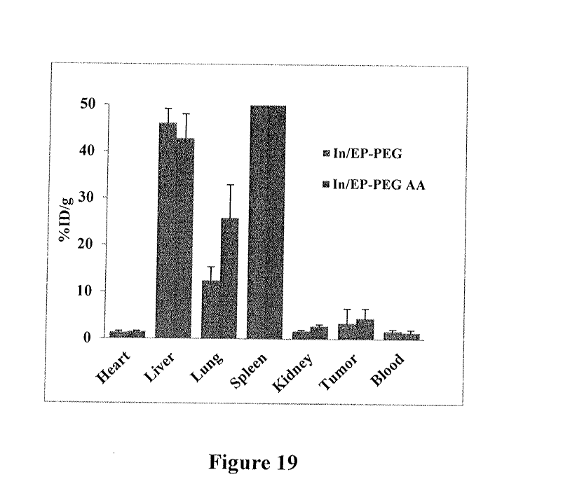

Systemic toxicity of nanoparticles was analyzed by histopathology of organs taken from mice treated with IEP nanoparticles. There are no indicators of significant toxicity observed in organs from treated mice. The bio-distribution of IEP nanoparticles was studied after injecting them into H460 tumor-bearing mice. In order to track the NPs, a portion of the indium was replaced with the radionuclide indium (¹¹¹In) during NP preparation. Six hours post-injection, mice were sacrificed and the amount of ¹¹¹In was measured in different organs. 3-4% of the injected dose was detected in tumor tissue (FIG. 19).

### Systemic Administration of IEP Nanoparticles Inhibited the H460 Tumor Growth in Xenograft Bearing Animal Models:

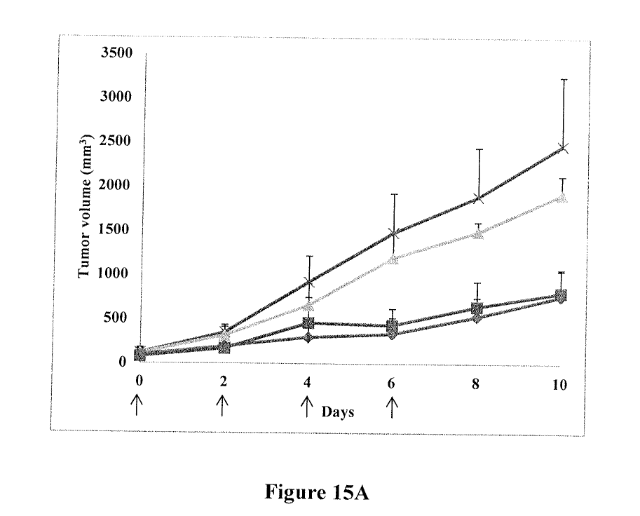

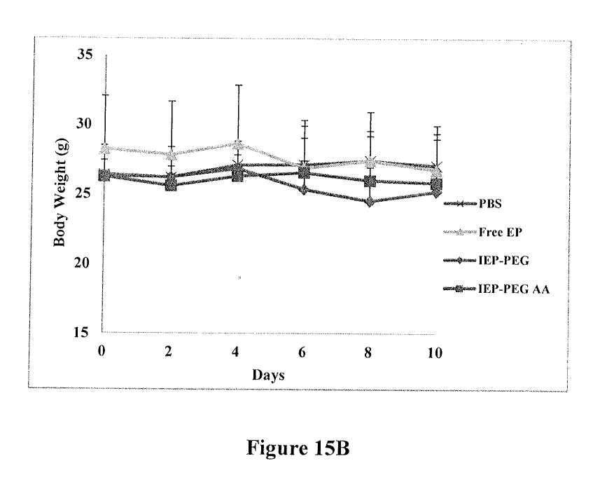

The pharmacodynamics of IEP nanoparticle formulations was evaluated in a mouse H460 xenograft tumor model. H460 xenograft bearing nude mice were intravenously injected with targeted and untargeted nanoparticles or free etoposide phosphate every other day for four days. Treatment with IEP nanoparticles significantly inhibited tumor growth relative to free drug or PBS (FIG. 15A). Nanoparticle delivery reduced the dosage of EP (5 mg/kg) needed to inhibit tumor growth, possibly due to enhanced accumulation of EP in tumor tissue. Free drug has a fast renal clearance profile, and has minimal impact on tumor growth (55). Body weight of treated mice did not change significantly as a result of treatment.

### IEP Nanoparticle Treatment Induced Apoptosis and Inhibited Tumor Cell Proliferation:

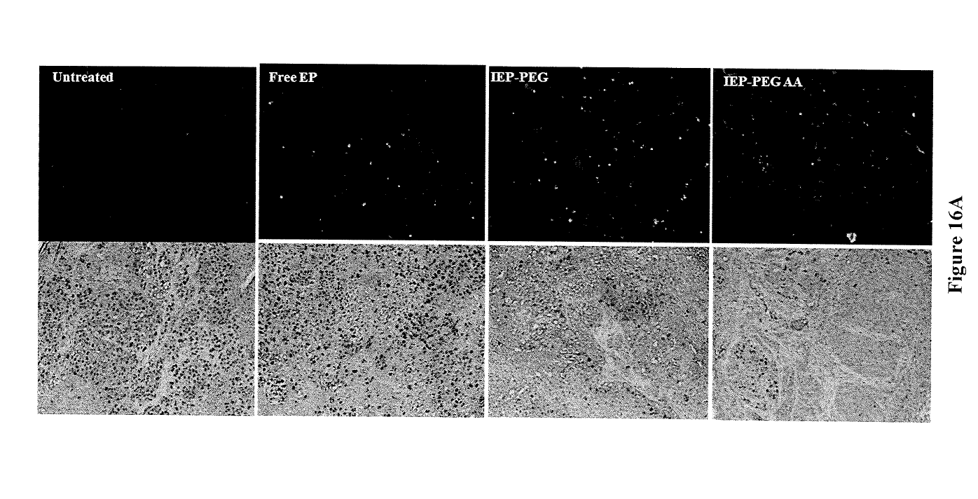

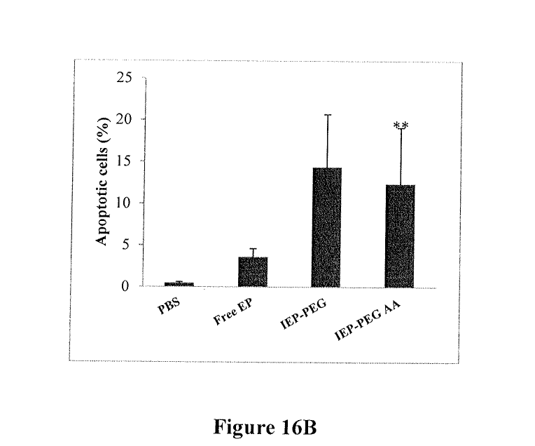

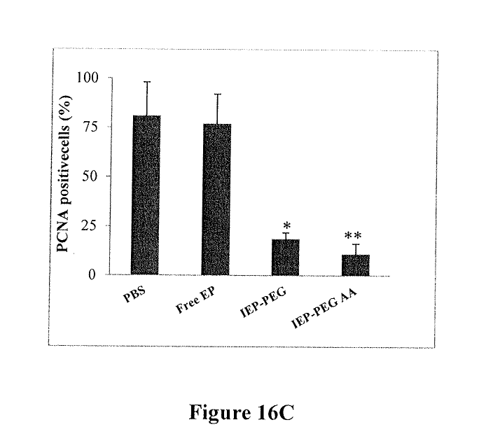

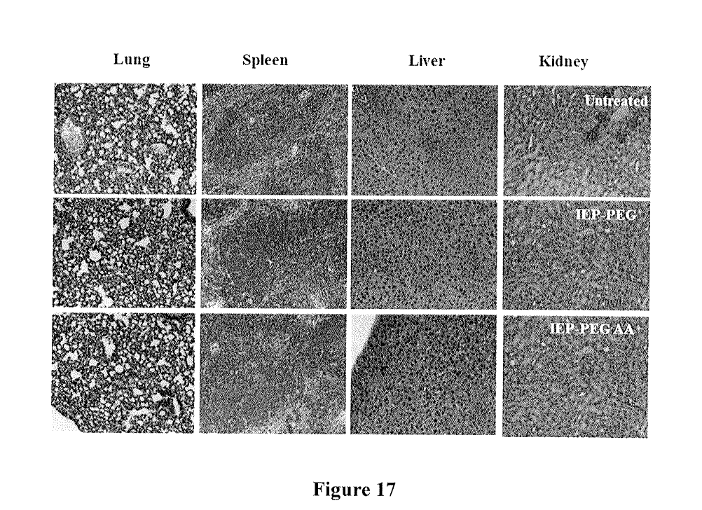

Tumor cell apoptosis was evaluated in vivo by Terminal deoxynucleotidyl transferase UTP nick end labeling (TUNEL) assay. The TUNEL assay is commonly used to detect fragmented DNA in apoptotic cells by using fluorescently labeled dUTP. Upon detection, apoptotic cells appear as dots of green fluorescence in tumor sections (FIG. 16, upper panel). The percentage of tumor cells that are apoptotic is significantly higher after treatment with targeted or untargeted nanoparticles but not free drug.

Proliferating cell nuclear antigen (PCNA) expression, known to be increased in actively proliferating cells, is used to evaluate the extent of cell proliferation in xenograft tumors after treatment (FIG. 16, lower panel). PCNA was detected by immunohistochemistry (FIG. 16B) in 75-80% of cells in tumor sections from untreated or free EP treated mice but only 10-20% of cells in mice treated with IEP nanoparticles. The nanoparticle system appears to effectively induce apoptosis and inhibit cell proliferation within tumors by increasing the bioavailability of systemically administered EP.

In an embodiment, an indium-based nanoparticle system for small cell lung cancer therapy is disclosed. Effective administration of the widely used anticancer drug, etoposide, is complicated by limitations stemming from its hydrophobicity, lack of solubility, and toxicity. The water-soluble analog, etoposide phosphate (EP), offers some improvement but bioavailability and toxicity still remain a problem. Toward more effective administration of this drug, etoposide loaded nanoparticles have been described but most reports are based on in vitro study of cultured cancer cell lines. Surprisingly, it is found that etoposide phosphate is able to co-precipitate with indium. Here indium is used as a carrier for, etoposide phosphate, to evaluate delivery of this water soluble prodrug (etoposide phosphate) to SCLC tumor cells both in vitro and in vivo. DOPA stabilized IEP core nanoparticles are synthesized using a micro emulsion method and then characterized in terms of shape and size.

Core particles are spherical and 45 nm in diameter, when coated with an outer layer of lipid and PEG increased diameter by about 5 nm. Anisamide modified PEG increased the zeta potential because of the positively-charged anisamide targeting ligand (81). Etoposide and etoposide phosphate can be degraded through epimerization of the lactone ring (85), but surprisingly, the formulation described herein did not alter etoposide phosphate structure, as confirmed by UV and ESI-MS (FIG. 12). Because surface functionality determines the fate of circulating nanoparticles in vivo, the nanoparticle surface is modified with PEG, a widely-used hydrophilic polymer, to permit escape from macrophage phagocyte system (MPS) uptake (63) resulting in prolonged time in circulation. Cellular uptake results suggest that the nanoparticles can target tumor cells through sigma receptors on the cell surface (81). Cytotoxicity studies in NCI-H460 lung cancer cell lines suggest that these nanoparticles efficiently deliver EP to tumor cells and exhibit dose-dependent anti-tumor activity. It is believed that, after endocytosis, released EP is converted to etoposide by intracellular phosphatases to achieve its cytotoxic effects (86) while no significant cytotoxicity is observed with free indium chloride, supporting the possibility of safe clinical use.

Anti-cancer effects were evaluated in an H460 xenograft-bearing mouse tumor model. Both targeted and untargeted IEP nanoparticles exhibit anti-tumor activity in mice (FIG. 15), any significant difference between targeted and untargeted nanoparticles has not been found. As shown, the cellular uptake of nanoparticles was receptor specific in culture (FIG. 13), and the in vitro results may predict similar effects in vivo (87, 88). PEGylated nanoparticles did accumulate in xenograft tumors although the targeting ligand did not appear to influence their bio-distribution or therapeutic efficacy over untargeted nanoparticles. AA may still facilitate cellular internalization of the nanoparticles through receptor mediated endocytosis (90, 91).

The anti-cancer prodrug etoposide phosphate was successfully encapsulated with a lipsome, which is 100 fold less toxic than etoposide, using an indium-based nanoparticle system. In vitro cytotoxicity studies and in vivo antitumor experiments reveal the efficacy of this nanoparticle formulation for successful delivery of the anticancer drug EP. Additionally, use of the radionuclide ¹¹¹In would be an excellent system for SPECT imaging, with possible simultaneous delivery of imaging agents and anti-cancer drugs with a single nanoparticle system. Other anti-cancer drugs such as cisplatin and gemcitabine can be encapsulated using the current technology. Accordingly, combined delivery of cisplatin with EP or gemcitabine with EP using the present nanoparticle system is possible, potentially as an effective therapy for lung cancer.

### Example 6

### Liposome-Encapsulated Cisplatin Nanoparticles-Neighboring Effect and Enhanced Anticancer Efficacy

a. Preparations of LPC NPs

LPC NPs were synthesized.¹¹⁸ Briefly, 200 mM cis-[Pt(NH₃)₂(H₂O)₂](NO₃)₂ and 800 mM KCl in water were separately dispersed in a solution composed of Cyclohexane/Igepal CO-520 (71:29, V:V) and Cyclohexane/Triton-X100/Hexanol (75:15:10, V:V:V) (3:1) to form a well-dispersed, water-in-oil reverse micro-emulsion. One hundred μL DOPA (20 mM) is added to the CDDP precursor phase and the mixture was stirred. The two emulsions were mixed for another 30 min while the reaction proceeded. Ethanol was added to the micro-emulsion and the particles were collected by centrifugation at 12,000 g. After being extensively washed with ethanol 2-3 times, the pellets were re-dispersed in 3.0 ml of chloroform and stored in a glass vial for further modification. Finally, 1.0 mL of LPC NPs core, 50 μL of 20 mM DOTAP, 50 μL of 20 mM Cholesterol and 50 μL of 10 mM DSPE-PEG-2000 or DSPE-PEG-AA were combined. After evaporating the chloroform, the residual lipids are dispersed in 1.0 mL of d-H₂O. The particle size of LPC NPs was determined using a Malvern ZetaSizer Nano series (Westborough, MA). TEM images of LPC NPs were acquired using a JEOL 100CX II TEM (JEOL, Japan). The LPC NPs were negatively stained with 2% uranyl acetate.

b. Preparations of DiI Labeled LPC NPs

DiI labeled LPC NPs were prepared in a method similar to the preparation of LPC NPs. Briefly, a mixture containing 1.0 mL of LPC NPs core, 50 μL of 20 mM DOTAP, 50 μL of 20 mM Cholesterol, 50 μL of 10 mM DSPE-PEG-2000 or DSPE-PEG-AA and 50 μL 1 mM DiI were combined. After evaporating the chloroform, the residual lipids were dispersed in 1.0 mL of d-H₂O.

c. Cell Toxicity Assay

A375M cells were seeded in 96-well plates at a density of 2000 cells/well and incubated in 10% FBS of DMEM containing 100 U/mL penicillin and 100 mg/mL streptomycin for 20 h. The medium was removed and replaced by Opti-MEM containing CDDP or LPC NPs. Forty-eight hours later, a CellTiter 96 AQueous One Solution Cell Proliferation Assay (Promega, Madison, Wis.) kit containing the tetrazolium compound MTS was used to assay cell viability according to the manufacturer's protocols. The IC₅₀ values were calculated using Graphpad Prism 5 (Graphpad Software Inc.)

d. Cellular Uptake

A375M cells (2×10⁵) were seeded in 35 mm, glass-bottom dishes (MatTek Corporation, Mass.) 20 h before the experiments began. The cells were treated with LPC NPs labeled with NBD-PE at a concentration of 100 μM Pt at 37° C. for 4 h. The cells were washed twice with PBS. The nucleus was stained with Hoechest 33342 (Sigma, St Louis, Mo.), and lysosomes were stained by lysotracker red (Invitrogen, Carlsbad, Calif.). The sample was observed using an Olympus FV 1000-MPE microscope (Olympus, Japan).

e. In Vitro Drug Release in 50% FBS

A suspension of LPC NPs containing 200 μg Pt in 50% FBS was incubated at 37° C. on a shaker at 300 rpm. During different time points, the corresponding samples were centrifuged at 16, 000 g for 20 min and the platinum drug released into the supernatant liquid was measured.

f. Cellular Release of Pt drug and Its Cell Toxicity

A375M cells were seeded in 24-well plates at a density of 3×10⁴ cells per well and incubated for 20 h in 10% FBS of DMEM containing 100 U/mL penicillin, and 100 mg/mL streptomycin. The medium was then removed and replaced by 100 tM of Opti-MEM containing CDDP or LPC NPs. All transfections were performed in triplicate. After incubation for 4 h at 37° C. in a 5% CO₂, humidified atmosphere, the medium was aspirated. Cells were washed and lysed in order to determine their uptake of NPs. The amount of Pt in cells was measured using ICP-MS. For the study of cellular release of Pt drug from cells, the medium was collected and replaced with fresh, completed medium at different time points. The intact NPs and free drug released into the medium were separated by centrifugation at 16,000 g for 20 min. The amount of Pt in the supernatant and pellets was measured using ICP-MS. To evaluate the toxicity of released drugs, the medium was transferred and incubated with untreated cells. Forty-eight hours later, a CellTiter 96 AQueous One Solution Cell Proliferation Assay (Promega, Madison, Wis.) kit containing the tetrazolium compound MTS was used to assay cell viability according to the manufacturer's protocols.

g. Biodistribution

The mice were administered a single dose of 1.0 mg/kg Pt CDDP and LPC NPs. Each group contained five mice, which were sacrificed four hours following injection. Tissue samples were digested by concentrated nitric acid overnight at room temperature and processed according to the procedure reported previously the literature.119, 120 The concentration of Pt was measured using ICP-MS.

h. In Vivo Anticancer Efficacy

All procedures involving experimental mice are performed in accordance with the protocols conformed to the Guide for the Care and Use of Laboratory Animals (NIH publication No. 86-23, revised 1985). Female athymic nude mice, 5-6 weeks old and weighing 18-22 g were obtained. 5×10⁶ A375M cells were injected subcutaneously into the mice. After 10 days, the mice were randomly divided into four groups (4-6 mice per group). The mice were treated with weekly IV injections of CDDP and LPC NPs and saline as a control. A dose of 1.0 mg/kg Pt was administered. Thereafter, tumor growth and body weight were monitored. Tumor volume was calculated using the following formula: TV=(L×W²)/2, with W being smaller than L. Finally, mice were sacrificed using a CO₂ inhalation method.

After the therapeutic experiment was complete, blood samples were collected and allowed to clot for 2 h at room temperature. Serum was obtained through centrifugation for 20 min at 2,000 g. For liver and renal function experiments, the levels of aspartate aminotransferase, alanine aminotransferase, and blood urea nitrogen in the serum were measured. Major organs were collected after treatment and were formalin fixed and processed for routine H&E staining using standard methods. Images were collected using a Nikon light microscope (Nikon). After the A375M tumor reached 600 mm³, the mice were treated with two weekly IV injections of LPC NPs at a dose of 3.0 mg/kg Pt. Seven days post the last injection, the mice were sacrificed and the tumors are assayed with TUNEL.

i. TUNEL Assay

The tumors were fixed in 4.0% paraformaldehyde (PFA), paraffin-embedded, and sectioned. To detect apoptotic cells in tumor tissues, a TUNEL assay, using a DeadEnd™ Fluorometric TUNEL System (Promega, Madison, Wis.), was performed following the manufacturer's protocols. Cell nuclei that were fluorescently stained with green were defined as TUNEL-positive nuclei. TUNEL-positive nuclei were monitored by using a fluorescence microscope (Nikon, Tokyo, Japan). The cell nuclei were stained with 4,6-diaminidino-2-phenyl-indole (DAPI) Vectashield (Vector Laboratories, Inc., Burlingame, Calif.). TUNEL-positive cells in three slides of images taken at 40× magnification were counted to quantify apoptosis.

j. In Vivo Neighboring Effect Study

In order to study the neighboring effect, the LPC NPs were labeled with DiI dye (Sigma-Aldrich, St Louis, Mo.) and administered to nude mice bearing A375M tumors at a single dose of 1.0 mg/kg Pt. Each group contained three mice that were sacrificed 24 h post injection. The organs and tumor sections were prepared by the procedure described in the TUNEL assay in supporting information. The distribution of NPs (red) and TUNEL positive cells (green) were observed using a fluorescence microscope (Nikon, Tokyo, Japan). The distance between two cells was measured using the NIS-Elements Microscope Imaging Software (Nikon Corp., Tokyo, Japan).

In order to observe the distribution of LPC NPs in liver, the sections were incubated with a 1:250 dilution of CD68 primary antibody (Abcam, Cambridge, Mass.) at 4° C. overnight followed by incubation with FITC-labeled secondary antibody (1:200, Santa Cruz, Calif.) for 1 h at room temperature. The sections were also stained by DAPI and covered with a coverslip. The sections were observed using a Nikon light microscope (Nikon Corp., Tokyo, Japan).

The CDDP-DNA adducts were detected using anti-CDDP modified DNA antibodies [CP9/19] (Abcam, Cambridge, Mass.). The sections were incubated with a 1:250 dilution of anti-CDDP modified DNA antibody [CP9/19] at 4° C. overnight followed by incubation with FITC-labeled goat anti-(rat Ig) antibody (1:200, Santa Cruz, Calif.) for 1 h at room temperature. The sections were also stained by DAPI and covered with a coverslip. The sections were observed using a Nikon light microscope (Nikon Corp., Tokyo, Japan).

k. In Vitro Neighboring Effect Study

A375M-GFP cells (2×10⁵) were seeded in 6-well plates (Corning Inc., Corning, N.Y.) 20 h before the beginning of the experiments. The cells were first treated with CDDP and LPC NPs (50 μM Pt) at 37° C. for 4 h and then trypsinized. The A375M-GFP cells were mixed with A375M cells at the ratio of 1:10 (total cell number: 2×10⁵) and reseeded into 6-well plates. After culturing for 48 h, the cells were stained with Hoechst 33342 (Sigma, St Louis, Mo.) and Annexin V Alexa Fluor® 568 Conjugate (Invitrogen, Carlsbad, Calif.). Cells stained with Alexa Fluor® 568 Conjugate were observed with a fluorescence microscope (Nikon, Tokyo, Japan) and quantified using flow cytometry (Becton-Dickinson, Heidelberg, Germany). Results were processed using the Cellquest software (Becton-Dickinson).

### Example 7

### Liposome-Encapsulated Cisplatin Nanoparticles with Tunable Size and Surface Modification for Melanoma Cancer Therapy

a. Materials and Methods

Lipids were purchased from Avanti Polar Lipids (Alabaster, Ala.). Dulbecco's Modified Eagle Medium (DMEM), L-glutamine, penicillin Gsodium, streptomycin and fetal calf serum are purchased from Gibco®. DSPE-PEG-AA was synthesized in our laboratory as previously reported.³³ 1-Hexanol is purchased from Alfa Aesar. Igepal® CO-520, triton™ X-100, cyclohexane, cisplatin and silver nitrate were obtained from Sigma-Aldrich (St Louis, Mo.) without further purification.

b. Cell Lines

1205Lu cells were cultured in DMEM medium supplemented with 10% heat-inactivated fetal bovine serum (FBS), 20 mM of L-glutamine, 100 U/ml of penicillin Gsodium, and 100 mg/ml of streptomycin at 37° C. in an atmosphere of 5% CO₂ and 95% air.

c. Synthesis of cis-[Pt(NH₃)₂(H₂O)₂](NO₃)₂ Precursor

To a suspension of CDDP (60 mg, 0.20 mmol) in 1.0 ml water, AgNO3 (66.2 mg, 0.39 mmol) was added. The mixture was heated at 60° C. for 3 h and then stirred overnight in a flask protected from light with aluminum foil. Afterwards, the mixture was centrifuged at 16,000 rpm for 15 min to remove the AgCl precipitate. The solution was then filtered using a 0.2 μm syringe filter. The concentration of cis-[Pt(NH₃)₂(H₂O)₂](NO₃)₂ was measured using ICP-MS and adjusted to 200 mM.

d. Preparation of LPC NPs

The synthesis route of LPC NPs is described in Scheme 2. First, 100 μL of 200 mM cis-[Pt(NH₃)₂(H₂O)₂](NO₃)₂ was dispersed in a solution composed of mixture of cyclohexane/Igepal CO-520 (71:29, V:V) and cyclohexane/triton-X100/hexanol (75:15:10, V:V:V) to form a well-dispersed, water-in-oil reverse micro-emulsion. Another emulsion containing KC1 was prepared by adding 100 μL of 800 mM KCl in water into a separate 8.0 mL oil phase. One hundred μL of DOPA (20 mM) was added to the CDDP precursor phase and the mixture was stirred. Twenty minutes later, the two emulsions were mixed and the reaction proceeded for another 30 min. After that, 16.0 mL of ethanol was added to the micro-emulsion and the mixture was centrifuged at 12,000 g for at least 15 min to remove the cyclohexane and surfactants. After being extensively washed with ethanol 2-3 times, the pellets were re-dispersed in 3.0 ml of chloroform and stored in a glass vial for further modification.

To prepare the final NPs, 1.0 mL of LPC NPs core, 100 μL of 20 mM DOTAP/Cholesterol (molar ratio 1:1) and 50 μL of 10 mM DSPE-PEG-2000 or DSPE-PEG-AA were combined. After evaporating the chloroform, the residual lipids were dispersed in 1.0 mL of d-H₂O.

e. Characterization of NPs

The zeta potential and particle size of LPC NPs were determined using a Malvern ZetaSizer Nano series (Westborough, Mass.). TEM images were acquired using a JEOL 100CX II TEM (JEOL, Japan). The LPC NPs were negatively stained with 2% uranyl acetate. The drug-loading capacity and platinum content were measured using inductively coupled plasma mass spectrometry (ICP-MS). The composition of DOPA-coated CDDP NPs was studied using XPS (Kratos Axis Ultra DLD X-ray Photoelectron Spectrometer) and NMR (Varain Inova 400 NMR Spectrometer).

f. Cell Toxicity Assay

1205Lu cells were seeded in 96-well plates at a density of 2000 cells/well and incubated in 10% FBS of DMEM containing 100 U/mL penicillin, and 100 mg/mL streptomycin for 20 h. Then, the medium was removed and replaced by Opti-MEM containing CDDP or LPC NPs. Forty-eight hours later, a CellTiter 96 AQueous One Solution Cell Proliferation Assay (Promega, Madison, Wis.) kit containing the tetrazolium compound MTS was used to assay cell viability according to the manufacturer's protocols.

g. Cellular Uptake

1205Lu cells (2×10⁵) were seeded in 35 mm MatTek glass bottom dishes (MatTek Corporation, Mass.) 20 h before experiments. The cells were treated with NBD-PE labeled LPC NPs at a concentration of 100 μM Pt at 37° C. for 4 h. The cells were subsequently washed twice with PBS. The nucleus was stained with Hoechest 33342 (Sigma, St Louis, Mo.), and lysosomes were stained by lysotracker red (Invitrogen, Carlsbad, Calif.). Then, the sample was observed by Olympus FV 1000-MPE microscope (Olympus, Japan). To measure the amount of Pt in cells, cells were washed and lysed in order to determine their uptake of NPs. The amount of Pt in cells is measured using ICP-MS.

h. In Vivo Anticancer Efficacy Evaluation

Female athymic nude mice, 5-6 weeks old and weighing 18-22 g were obtained. 1205Lu xenograft tumors were developed through subcutaneous injection of approximately 5 million 1205Lu cells in female nude mice. 2.0 mg/kg of Pt was administered weekly by IV injection for CDDP and LPC NPs groups. Tumor growth and body weight were monitored. Tumor volume was calculated using the following formula: TV=(L×W²)/2, with W being smaller than L. Finally, mice were sacrificed by CO₂ inhalation. Tumors were collected after treatment and were formalin fixed and processed for TUNEL assay.

i. TUNEL Assay

The tumors were fixed in 4.0% paraformaldehyde (PFA) and subsequently paraffin-embedded and sectioned. To detect apoptotic cells in tumor tissues, a TUNEL assay, using a DeadEnd™ Fluorometric TUNEL System (Promega, Madison, Wis.), was performed, following the manufacturer's protocol. Cell nuclei, which were stained with green fluorescence, are defined as TUNEL-positive nuclei. TUNEL-positive nuclei were monitored using a fluorescence microscope (Nikon, Tokyo, Japan). The cell nuclei were stained with 4, 6-diaminidino-2-phenyl-indole (DAPI) (Vectashield, Vector Laboratories, Inc., Burlingame, Calif.). To quantify TUNEL-positive cells, green-fluorescence-positive cells are counted in three images taken at 40× magnification.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the foregoing list of embodiments and appended claims. Although specific terms are employed herein, they are used in a generic and descriptive sense only and not for purposes of limitation.

### BIBLIOGRAPHY

1. Veal, G. J., Griffin, M. J., Price, E., Parry, A., Dick, G. S., Little, M. A., Yule, S. M., Morland, B., Estlin, E. J., Hale, J. P. et al. (2001) A phase I study in pediatric patients to evaluate the safety and pharmacokinetics of SPI-77, a liposome encapsulated formulation of cisplatin. Br J Cancer, 84, 1029-1035.
2. Nishiyama, N., Okazaki, S., Cabral, H., Miyamoto, M., Kato, Y., Sugiyama, Y., Nishio, K., Matsumura, Y. and Kataoka, K. (2003) Novel Cisplatin-Incorporated Polymeric Micelles Can Eradicate Solid Tumors in Mice. Cancer Research, 63, 8977-8983.
3. Sengupta, P., Basu, S., Soni, S., Pandey, A., Roy, B., Oh, M. S., Chin, K. T., Paraskar, A. S., Sarangi, S., Connor, Y. et al. (2012) Cholesterol-tethered platinum II-based supramolecular nanoparticle increases antitumor efficacy and reduces nephrotoxicity. Proc Natl Acad Sci USA, 109, 11294-11299.
4. Pabla, N. and Dong, Z. (2008) Cisplatin nephrotoxicity: mechanisms and renoprotective strategies. Kidney Int, 73, 994-1007.
5. Safirstein, R., Winston, J., Goldstein, M., Moel, D., Dikman, S. and Guttenplan, J. (1986) Cisplatin nephrotoxicity. Am J Kidney Dis, 8, 356-367.
6. Yao, X., Panichpisal, K., Kurtzman, N. and Nugent, K. (2007) Cisplatin nephrotoxicity: a review. Am J Med Sci, 334, 115-124.
7. Huang, L. and Liu, Y. (2011) In Vivo Delivery of RNAi with Lipid-Based Nanoparticles. Annual Review of Biomedical Engineering, 13, 507-530.
8. Kim, S. K., Foote, M. B. and Huang, L. Targeted delivery of EV peptide to tumor cell cytoplasm using lipid coated calcium carbonate nanoparticles. Cancer Letters.
9. Li, J., Chen, Y.-C., Tseng, Y.-C., Mozumdar, S. and Huang, L. (2010) Biodegradable calcium phosphate nanoparticle with lipid coating for systemic siRNA delivery. Journal of Controlled Release, 142, 416-421.
10. Li, J., Yang, Y. and Huang, L. (2012) Calcium phosphate nanoparticles with an asymmetric lipid bilayer coating for siRNA delivery to the tumor. Journal of Controlled Release, 158, 108-114.
11. Yang, Y., Li, J., Liu, F. and Huang, L. (2012) Systemic Delivery of siRNA via LCP Nanoparticle Efficiently Inhibits Lung Metastasis. Mol Ther, 20, 609-615.
12. Yang, Y., Hu, Y., Wang, Y., Li, J., Liu, F. and Huang, L. (2012) Nanoparticle Delivery of Pooled siRNA for Effective Treatment of Non-Small Cell Lung Cancer. Molecular Pharmaceutics, 9, 2280-2289.
13. Dhar, S., Kolishetti, N., Lippard, S. J. and Farokhzad, O. C. (2011) Targeted delivery of a cisplatin prodrug for safer and more effective prostate cancer therapy in vivo. Proc Natl Acad Sci U S A, 108, 1850-1855.
14. Boulikas, T. (2009) Clinical overview on Lipoplatin™: a successful liposomal formulation of cisplatin. Expert Opinion on Investigational Drugs, 18, 1197-1218.
15. Grinberg, A. A. and Dobroborskaya, A. I. (1967) Water solubility of isomeric diammineplatinum compounds. Zh. Neorg. Khim., 12, 276-277.
16. Seetharamu, N., Kim, E., Hochster, H., Martin, F. and Muggia, F. (2010) Phase II study of liposomal cisplatin (SPI-77) in platinum-sensitive recurrences of ovarian cancer. Anticancer Res, 30, 541-545.
17. Wang, A. Z., Langer, R. and Farokhzad, O. C. (2012) Nanoparticle delivery of cancer drugs. Annu Rev Med, 63, 185-198.
18. Li, S. D., Chen, Y. C., Hackett, M. J. and Huang, L. (2008) Tumor-targeted delivery of siRNA by self-assembled nanoparticles. Mol Ther, 16, 163-169.
19. Cabral, H., Matsumoto, Y., Mizuno, K., Chen, Q., Murakami, M., Kimura, M., Terada, Y., Kano, M.R., Miyazono, K., Uesaka, M. et al. (2011) Accumulation of sub-100 nm polymeric micelles in poorly permeable tumours depends on size. Nat Nanotechnol, 6, 815-823.
20. Lu, C., Perez-Soler, R., Piperdi, B., Walsh, G. L., Swisher, S. G., Smythe, W. R., Shin, H. J., Ro, J. Y., Feng, L., Truong, M. et al. (2005) Phase II study of a liposome-entrapped cisplatin analog (L-NDDP) administered intrapleurally and pathologic response rates in patients with malignant pleural mesothelioma. J Clin Oncol, 23, 3495-3501.
21. Vail, D. M., Kurzman, I. D., Glawe, P. C., O'Brien, M. G., Chun, R., Garrett, L. D., Obradovich, J. E., Fred, R. M., 3rd, Khanna, C., Colbern, G. T. et al. (2002) STEALTH liposome-encapsulated cisplatin (SPI-77) versus carboplatin as adjuvant therapy for spontaneously arising osteosarcoma (OSA) in the dog: a randomized multicenter clinical trial. Cancer Chemother Pharmacol, 50, 131-136.
22. Harrington, K. J., Rowlinson-Busza, G., Syrigos, K. N., Vile, R. G., Uster, P. S., Peters, A. M. and Stewart, J. S. (2000) Pegylated liposome-encapsulated doxorubicin and cisplatin enhance the effect of radiotherapy in a tumor xenograft model. Clin Cancer Res, 6, 4939-4949.
23. Harrington, K. J., Rowlinson-Busza, G., Uster, P. S. and Stewart, J. S. (2000) Pegylated liposome-encapsulated doxorubicin and cisplatin in the treatment of head and neck xenograft tumours. Cancer Chemother Pharmacol, 46, 10-18.
24. Thamm, D. H. and Vail, D. M. (1998) Preclinical evaluation of a sterically stabilized liposome-encapsulated cisplatin in clinically normal cats. Am J Vet Res, 59, 286-289.
25. Khiati, S., Luvino, D., Oumzil, K., Chauffert, B., Camplo, M. and Barthelemy, P. (2011) Nucleoside-Lipid-Based Nanoparticles for Cisplatin Delivery. ACS Nano, 5, 8649-8655.
26. Zamboni, W., Gervais, A., Egorin, M., Schellens, J. M., Zuhowski, E., Pluim, D., Joseph, E., Hamburger, D., Working, P., Colbern, G. et al. (2004) Systemic and tumor disposition of platinum after administration of cisplatin or STEALTH liposomal-cisplatin formulations (SPI-077 and SPI-077 B 103) in a preclinical tumor model of melanoma. Cancer Chemotherapy and Pharmacology, 53, 329-336.
27. Zisman, N., Dos Santos, N., Johnstone, S., Tsang, A., Bermudes, D., Mayer, L. and Tardi, P. (2011) Optimizing Liposomal Cisplatin Efficacy through Membrane Composition Manipulations. Chemotherapy Research and Practice, 2011.
28. Baba, M., Matsumoto, Y., Kashio, A., Cabral, H., Nishiyama, N., Kataoka, K. and Yamasoba, T. (2012) Micellization of cisplatin (NC-6004) reduces its ototoxicity in guinea pigs. J Control Release, 157, 112-117.
29. Plummer, R., Wilson, R.H., Calvert, H., Boddy, A. V., Griffin, M., Sludden, J., Tilby, M. J., Eatock, M., Pearson, D. G., Ottley, C. J. et al. (2011) A Phase I clinical study of cisplatin-incorporated polymeric micelles (NC-6004) in patients with solid tumours. Br J Cancer, 104, 593-598.
30. Sengupta, P., Basu, S., Soni, S., Pandey, A., Roy, B., Oh, M. S., Chin, K. T., Paraskar, A. S., Sarangi, S., Connor, Y. et al. (2012) Cholesterol-tethered platinum II-based supramolecular nanoparticle increases antitumor efficacy and reduces nephrotoxicity. Proceedings of the National Academy of Sciences, 109, 11294-11299.
31. Society, A. C. (2011) Cancer Facts & Figures. 2011. American Cancer Society, Atlanta.
32. Sertoli, M. R., Queirolo, P., Bajetta, E., Del Vecchio, M., Comella, G., Barduagni, L., Bernengo, M. G., Vecchio, S., Criscuolo, D., Bufalino, R. et al. (1999) Multi-institutional phase II randomized trial of integrated therapy with cisplatin, dacarbazine, vindesine, subcutaneous interleukin-2, interferon alpha2a and tamoxifen in metastatic melanoma. BREMIM (Biological Response Modifiers in Melanoma). Melanoma Res, 9, 503-509.
33. Shahzad, M. M. K., Lopez-Berestein, G. and Sood, A. K. (2009) Novel strategies for reversing platinum resistance. Drug Resistance Updates, 12, 148-152.
34. Caterina, A. M. L. P. (2007) Drug Resistance in Melanoma: New Perspectives. Current Medicinal Chemistry, 14, 387-391.
35. Helmbach, H., Rossmann, E., Kern, M. A. and Schadendorf, D. (2001) Drug-resistance in human melanoma. International Journal of Cancer, 93, 617-622.
36. Mandic, A., Viktorsson, K., Heiden, T., Hansson, J. and Shoshan, M. C. (2001) The MEK1 inhibitor PD98059 sensitizes C8161 melanoma cells to cisplatin-induced apoptosis. Melanoma Research, 11, 11-19.
37. Helmbach, H., Kern, M. A., Rossmann, E., Renz, K., Kissel, C., Gschwendt, B. and Schadendorf, D. (2002) Drug Resistance Towards Etoposide and Cisplatin in Human Melanoma Cells is Associated with Drug-Dependent Apoptosis Deficiency. 118, 923-932.
38. Runger, T. M., Emmert, S., Schadendorf, D., Diem, C., Epe, B. and Hellfritsch, D. (2000) Alterations of DNA Repair in Melanoma Cell Lines Resistant to Cisplatin, Fotemustine, or Etoposide. J Investig Dermatol, 114, 34-39.
39. Liedert, B., Materna, V., Schadendorf, D., Thomale, J. and Lage, H. (2003) Overexpression of cMOAT (MRP2/ABCC2) Is Associated with Decreased Formation of Platinum-DNA Adducts and Decreased G2-Arrest in Melanoma Cells Resistant to Cisplatin. J Investig Dermatol, 121, 172-176.
40. Gottesman, M. M., Fojo, T. and Bates, S. E. (2002) Multidrug resistance in cancer: role of ATP-dependent transporters. Nat Rev Cancer, 2, 48-58.
41. Shen, D.-w., Pastan, I. and Gottesman, M. M. (1998) Cross-Resistance to Methotrexate and Metals in Human Cisplatin-resistant Cell Lines Results from a Pleiotropic Defect in Accumulation of These Compounds Associated with Reduced Plasma Membrane Binding Proteins. Cancer Research, 58, 268-275.
42. Shen, D.-W., Goldenberg, S., Pastan, I. and Gottesman, M. M. (2000) Decreased accumulation of [14c]carboplatin in human cisplatin-resistant cells results from reduced energy-dependent uptake. Journal of Cellular Physiology, 183, 108-116.
43. Taniguchi, K., Wada, M., Kohno, K., Nakamura, T., Kawabe, T., Kawakami, M., Kagotani, K., Okumura, K., Akiyama, S.-i. and Kuwano, M. (1996) A Human Canalicular Multispecific Organic Anion Transporter (cMOAT) Gene Is Overexpressed in Cisplatin-resistant Human Cancer Cell Lines with Decreased Drug Accumulation. Cancer Research, 56, 4124-4129.
44. Parker, R. J., Eastman, A., Bostick-Bruton, F. and Reed, E. (1991) Acquired cisplatin resistance in human ovarian cancer cells is associated with enhanced repair of cisplatin-DNA lesions and reduced drug accumulation. The Journal of Clinical Investigation, 87, 772-777.
45. Loh, S. Y., Mistry, P., Kelland, L. R., Abel, G. and Harrap, K. R. (1992) Reduced drug accumulation as a major mechanism of acquired resistance to cisplatin in a human ovarian carcinoma cell line: circumvention studies using novel platinum (II) and (IV) ammine/amine complexes. Br J Cancer, 66, 1109-1115.
46. Liang, X.-J., Meng, H., Wang, Y., He, H., Meng, J., Lu, J., Wang, P. C., Zhao, Y., Gao, X., Sun, B. et al. (2010) Metallofullerene nanoparticles circumvent tumor resistance to cisplatin by reactivating endocytosis. Proceedings of the National Academy of Sciences, 107, 7449-7454.
47. Deng, W.-j., Yang, X.-q., Liang, Y.-j., Chen, L.-m., Yan, Y.-y., Shuai, X.-t. and Fu, L.-w. (2007) FG020326-loaded nanoparticle with PEG and PDLLA improved pharmacodynamics of reversing multidrug resistance in vitro and in vivol. Acta Pharmacologica Sinica, 28, 913-920.
48. Shapira, A., Livney, Y. D., Broxterman, H. J. and Assaraf, Y. G. (2011) Nanomedicine for targeted cancer therapy: Towards the overcoming of drug resistance. Drug Resistance Updates, 14, 150-163.
49. Chen, B., Sun, Q., Wang, X., Gao, F., Dai, Y., Yin, Y., Ding, J., Gao, C., Cheng, J., Li, J. et al. (2008) Reversal in multidrug resistance by magnetic nanoparticle of Fe3O4 loaded with adriamycin and tetrandrine in K562/A02 leukemic cells. Int J Nanomedicine, 3, 277-286.
50. Johannessen, C. M., Boehm, J. S., Kim, S. Y., Thomas, S. R., Wardwell, L., Johnson, L. A., Emery, C. M., Stransky, N., Cogdill, A. P., Barretina, J. et al. (2010) COT drives resistance to RAF inhibition through MAP kinase pathway reactivation. Nature, 468, 968-972.
51. Alcala, A. M. and Flaherty, K. T. (2012) BRAF inhibitors for the treatment of metastatic melanoma: clinical trials and mechanisms of resistance. Clin Cancer Res, 18, 33-39.
52. Chapman, P. B., Hauschild, A., Robert, C., Haanen, J. B., Ascierto, P., Larkin, J., Dummer, R., Garbe, C., Testori, A., Maio, M. et al. (2011) Improved survival with vemurafenib in melanoma with BRAF V600E mutation. N Engl J Med, 364, 2507-2516.
53. Flaherty, K. T., Puzanov, I., Kim, K. B., Ribas, A., McArthur, G. A., Sosman, J. A., O'Dwyer, P. J., Lee, R. J., Grippo, J. F., Nolop, K. et al. (2010) Inhibition of mutated, activated BRAF in metastatic melanoma. N Engl J Med, 363, 809-819.
54. Kelland, L. R., Sharp, S. Y., O'Neill, C. F., Raynaud, F. I., Beale, P. J. and Judson, I. R. (1999) Mini-review: discovery and development of platinum complexes designed to circumvent cisplatin resistance. J Inorg Biochem, 77, 111-115.
55. Hande K R. Etoposide: four decades of development of a topoisomerase II inhibitor. Eur J Cancer. 1998;34(10):1514-21. Epub 1999/01/20.
56. Larsen A K, Skladanowski A, Bojanowski K. The roles of DNA topoisomerase II during the cell cycle. Progress in cell cycle research. 1996; 2:229-39. Epub 1996/01/01.
57. Fields S Z, Igwemezie L N, Kaul S, Schacter L P, Schilder R J, Litam P P, et al. Phase I study of etoposide phosphate (etopophos) as a 30-minute infusion on days 1, 3, and 5. Clinical cancer research : an official journal of the American Association for Cancer Research. 1995;1(1):105-11. Epub 1995/01/01.
58. Rose W C, Basler G A, Trail P A, Saulnier M, Crosswell A R, Casazza A M. Preclinical antitumor activity of a soluble etoposide analog, BMY-40481-30. Investigational new drugs. 1990; 8 Suppl 1:S25-32. Epub 1990/01/01.
59. Senter P D, Saulnier M G, Schreiber G J, Hirschberg D L, Brown J P, Hellstrom I, et al. Anti-tumor effects of antibody-alkaline phosphatase conjugates in combination with etoposide phosphate. Proceedings of the National Academy of Sciences of the United States of America. 1988; 85(13):4842-6. Epub 1988/07/01.
60. Heath J R, Davis M E. Nanotechnology and cancer. Annual review of medicine. 2008; 59:251-65. Epub 2007/10/17.
61. Cancer nanotechnology: small, but heading for the big time. Nature reviews Drug discovery. 2007; 6(3):174-5. Epub 2007/03/31.
62. Fang J, Nakamura H, Maeda H. The EPR effect: Unique features of tumor blood vessels for drug delivery, factors involved, and limitations and augmentation of the effect. Advanced drug delivery reviews. 2011; 63(3):136-51. Epub 2010/05/06.
63. Ramishetti S, Huang L. Intelligent design of multifunctional lipid-coated nanoparticle platforms for cancer therapy. Therapeutic delivery. 2012; 3(12):1429-45. Epub 2013/01/18.
64. Chen C, Xie X X, Zhou Q, Zhang F Y, Wang Q L, Liu Y Q, et al. EGF-functionalized single-walled carbon nanotubes for targeting delivery of etoposide. Nanotechnology. 2012; 23 (4): 045104. Epub 2012/01/10.
65. Qian W Y, Sun D M, Zhu R R, Du X L, Liu H, Wang S L. pH-sensitive strontium carbonate nanoparticles as new anticancer vehicles for controlled etoposide release. International journal of nanomedicine. 2012; 7:5781-92. Epub 2012/11/28.
66. Lamprecht A, Benoit J P. Etoposide nanocarriers suppress glioma cell growth by intracellular drug delivery and simultaneous P-glycoprotein inhibition. Journal of controlled release : official journal of the Controlled Release Society. 2006; 112(2):208-13. Epub 2006/04/01.
67. Kalhapure R S, Akamanchi K G. A novel biocompatible bicephalous dianionic surfactant from oleic acid for solid lipid nanoparticles. Colloids and surfaces B, Biointerfaces. 2013; 105:215-22. Epub 2013/02/05.
68. Callewaert M, Dukic S, Van Gulick L, Vittier M, Gafa V, Andry M C, et al. Etoposide encapsulation in surface-modified poly(lactide-co-glycolide) nanoparticles strongly enhances glioma antitumor efficiency. Journal of biomedical materials research Part A. 2013; 101(5):1319-27. Epub 2012/10/16.
69. Snehalatha M, Venugopal K, Saha R N, Babbar A K, Sharma R K. Etoposide loaded PLGA and PCL nanoparticles II: biodistribution and pharmacokinetics after radiolabeling with Tc-99m. Drug delivery. 2008; 15(5):277-87. Epub 2008/09/03.
70. Shah S, Pal A, Gude R, Devi S. A novel approach to prepare etoposide-loaded poly(N-vinyl caprolactam-co-methylmethacrylate) copolymeric nanoparticles and their controlled release studies. Journal of applied polymer science. 2013; 127(6):4991-9.
71. Tang B C, Fu J, Watkins D N, Hanes J. Enhanced efficacy of local etoposide delivery by poly(ether-anhydride) particles against small cell lung cancer in vivo. Biomaterials. 2010; 31(2):339-44. Epub 2009/10/03.
72. Yarema M, Pichler S, Kriegner D, Stangl J, Yarema O, Kirchschlager R, et al. From highly monodisperse indium and indium tin colloidal nanocrystals to self-assembled indium tin oxide nanoelectrodes. ACS nano. 2012; 6(5):4113-21. Epub 2012/04/18.
73. Suzuki I, Kiyokawa K, Yasuda M, Baba A. Indium(III) Halide-Catalyzed UV-Irradiated Radical Coupling of Iodomethylphosphorus Compounds with Various Organostannanes. Organic letters. 2013. Epub 2013/03/23.
74. Psimadas D, Georgoulias P, Valotassiou V, Loudos G. Molecular nanomedicine towards cancer: (1)(1)(1)In-labeled nanoparticles. Journal of pharmaceutical sciences. 2012; 101(7):2271-80. Epub 2012/04/11.
75. Kelkar S S, Reineke T M. Theranostics: combining imaging and therapy. Bioconjugate chemistry. 2011; 22(10):1879-903. Epub 2011/08/13.
76. Kairemo K J, Ramsay H A, Tagesson M, Jekunen A P, Paavonen T K, Jaaskela-Saari H A, et al. Indium-111 bleomycin complex for radiochemotherapy of head and neck cancer—dosimetric and biokinetic aspects. European journal of nuclear medicine. 1996; 23(6):631-8. Epub 1996/06/01.
77. Kairemo K J, Ramsay H, Nikula T K, Hopsu E V, Taavitsainen M J, Bondestam S, et al. A low pH 111In-bleomycin complex: a tracer for radiochemotherapy of head and neck cancer. J Nucl Biol Med. 1994; 38(4 Suppl 1):135-9. Epub 1994/12/01.
78. Klibanov A L, Maruyama K, Beckerleg A M, Torchilin V P, Huang L. Activity of amphipathic poly(ethylene glycol) 5000 to prolong the circulation time of liposomes depends on the liposome size and is unfavorable for immunoliposome binding to target. Biochimica et biophysica acta. 1991; 1062(2):142-8. Epub 1991/02/25.
79. Petros R A, DeSimone J M. Strategies in the design of nanoparticles for therapeutic applications. Nature reviews Drug discovery. 2010; 9(8):615-27. Epub 2010/07/10.
80. Banerjee R, Tyagi P, Li S, Huang L. Anisamide-targeted stealth liposomes: a potent carrier for targeting doxorubicin to human prostate cancer cells. International journal of cancer Journal international du cancer. 2004; 112(4):693-700. Epub 2004/09/24.
81. Zhang Y, Kim W Y, Huang L. Systemic delivery of gemcitabine triphosphate via LCP nanoparticles for NSCLC and pancreatic cancer therapy. Biomaterials. 2013; 34(13):3447-58. Epub 2013/02/06.
82. Sleiman R J, Stewart B W. Early caspase activation in leukemic cells subject to etoposide-induced G2-M arrest: evidence of commitment to apoptosis rather than mitotic cell death. Clinical cancer research : an official journal of the American Association for Cancer Research. 2000; 6(9):3756-65. Epub 2000/09/22.
83. Chaitanya G V, Steven A J, Babu P P. PARP-1 cleavage fragments: signatures of cell-death proteases in neurodegeneration. Cell communication and signaling: CCS. 2010; 8:31. Epub 2010/12/24.
84. Smith P J, Soues S, Gottlieb T, Falk S J, Watson J V, Osborne R J, et al. Etoposide-induced cell cycle delay and arrest-dependent modulation of DNA topoisomerase II in small-cell lung cancer cells. British journal of cancer. 1994; 70(5):914-21. Epub 1994/11/01.
85. Beijnen J H, Holthuis J J M, Kerkdijk H G, Vanderhouwen O A G J, Paalman A C A, Bult A, et al. Degradation Kinetics of Etoposide in Aqueous-Solution. Int J Pharm. 1988; 41(1-2):169-78.
86. Karl D M, Craven D B. Effects of alkaline phosphatase activity on nucleotide measurements in aquatic microbial communities. Applied and environmental microbiology. 1980; 40(3):549-61. Epub 1980/09/01.
87. Damia G, D'Incalci M. Contemporary pre-clinical development of anticancer agents—what are the optimal preclinical models? Eur J Cancer. 2009; 45(16):2768-81. Epub 2009/09/19.
88. Mikhail A S, Allen C. Block copolymer micelles for delivery of cancer therapy: transport at the whole body, tissue and cellular levels. Journal of controlled release: official journal of the Controlled Release Society. 2009; 138(3):214-23. Epub 2009/04/21.
89. Iyer A K, Khaled G, Fang J, Maeda H. Exploiting the enhanced permeability and retention effect for tumor targeting. Drug discovery today. 2006; 11(17-18):812-8. Epub 2006/08/29.
90. Pirollo K F, Chang E H. Does a targeting ligand influence nanoparticle tumor localization or uptake? Trends in biotechnology. 2008; 26(10):552-8. Epub 2008/08/30.
91. Kwon I K, Lee S C, Han B, Park K. Analysis on the current status of targeted drug delivery to tumors. Journal of controlled release: official journal of the Controlled Release Society. 2012; 164(2):108-14. Epub 2012/07/18.
92. Rosenber. B; Vancamp, L.; Krigas, T., Inhibition of Cell Division in Escherichia Coli by Electrolysis Products from a Platinum Electrode. Nature 1965, 205, 698-699.
93. Lebwohl, D.; Canetta, R., Clinical Development of Platinum Complexes in Cancer Therapy: An Historical Perspective and an Update. Eur. J. Cancer 1998, 34, 1522-1534.
94. Drayton, R. M.; Catto, J. W. F., Molecular Mechanisms of Cisplatin Resistance in Bladder Cancer. Expert Rev. Anticanc. 2012, 12, 271-281.
95. Liang, X. J.; Meng, H.; Wang, Y.; He, H.; Meng, J.; Lu, J.; Wang, P. C.; Zhao, Y.; Gao, X.; Sun, B., et al., Metallofullerene Nanoparticles Circumvent Tumor Resistance to Cisplatin by Reactivating Endocytosis.Proc. Natl. Acad. Sci. U.S.A. 2010, 107, 7449-7454.
96. Go, R. S.; Adjei, A. A., Review of the Comparative Pharmacology and Clinical Activity of Cisplatin and Carboplatin. J. Clin. Oncol. 1999, 17, 409-422.
97. Farokhzad, O. C.; Langer, R., Nanomedicine: Developing Smarter Therapeutic and Diagnostic Modalities. Adv. Drug Deliv. Rev. 2006, 58, 1456-1459.
98. Davis, M. E.; Chen, Z.; Shin, D. M., Nanoparticle Therapeutics: An Emerging Treatment Modality for Cancer. Nat. Rev. Drug Discov. 2008, 7, 771-782.
99. Klibanov, A. L.; Maruyama, K.; Torchilin, V. P.; Huang, L., Amphipathic Polyethyleneglycols Effectively Prolong the Circulation Time of Liposomes. FEBS Lett. 1990, 268, 235-237.
100. Farokhzad, O. C.; Karp, J. M.; Langer, R., Nanoparticle-Aptamer Bioconjugates for Cancer Targeting. Expert Opin. Drug Del. 2006, 3, 311-324.
101. Banerjee, R.; Tyagi, P.; Li, S.; Huang, L., Anisamide-Targeted Stealth Liposomes: A Potent Carrier for Targeting Doxorubicin to Human Prostate Cancer Cells. Int. J. Cancer 2004, 112, 693-700.
102. Li, Z.; Huang, P.; Zhang, X.; Lin, J.; Yang, S.; Liu, B.; Gao, F.; Xi, P.; Ren, Q.; Cui, D., Rgd-Conjugated Dendrimer-Modified Gold Nanorods for in Vivo Tumor Targeting and Photothermal Therapy†. Mol. Pharm. 2009, 7, 94-104.
103. Zhang, C. F.; Jugold, M.; Woenne, E. C.; Lammers, T.; Morgenstern, B.; Mueller, M. M.; Zentgraf, H.; Bock, M.; Eisenhut, M.; Semmler, W., et al., Specific Targeting of

Tumor Angiogenesis by Rgd-Conjugated Ultrasmall Superparamagnetic Iron Oxide Particles Using a Clinical 1.5-T Magnetic Resonance Scanner. Cancer Res. 2007, 67, 1555-1562.
104. Maeda, H.; Wu, J.; Sawa, T.; Matsumura, Y.; Hori, K., Tumor Vascular Permeability and the Epr Effect in Macromolecular Therapeutics: A Review. J. Control. Release 2000, 65, 271-284.
105. Cabral, H.; Matsumoto, Y.; Mizuno, K.; Chen, Q.; Murakami, M.; Kimura, M.; Terada, Y.; Kano, M. R.; Miyazono, K.; Uesaka, M., et al., Accumulation of Sub-100 Nm Polymeric Micelles in Poorly Permeable Tumours Depends on Size. Nat. Nano. 2011, 6, 815-823.
106. Chauhan, V. P.; Stylianopoulos, T.; Martin, J. D.; Popovié, Z.; Chen, O.; Kamoun, W. S.; Bawendi, M. G.; Fukumura, D.; Jain, R. K., Normalization of Tumour Blood Vessels Improves the Delivery of Nanomedicines in a Size-Dependent Manner. Nat. Nano. 2012, 7, 383-388.
107. Burger, K. N.; Staffhorst, R. W.; de Vijlder, H. C.; Velinova, M. J.; Bomans, P. H.; Frederik, P. M.; de Kruijff, B., Nanocapsules: Lipid-Coated Aggregates of Cisplatin with High Cytotoxicity. Nat. Med. 2002, 8, 81-84.
108. Hamelers, I. H.; de Kroon, A. I., Nanocapsules: A Novel Lipid Formulation Platform for Platinum-Based Anti-Cancer Drugs. J. Liposome Res. 2007, 17, 183-189.
109. Khiati, S.; Luvino, D.; Oumzil, K.; Chauffert, B.; Camplo, M.; Barthelemy, P., Nucleoside-Lipid-Based Nanoparticles for Cisplatin Delivery. ACS Nano 2011, 5, 8649-8655.
110. Kieler-Ferguson, H. M.; Fréchet, J. M. J.; Szoka Jr, F. C., Clinical Developments of Chemotherapeutic Nanomedicines: Polymers and Liposomes for Delivery of Camptothecins and Platinum (Ii) Drugs. Wiley Interdiscip. Rev. Nanomed. Nanobiotechnol. 2013, 5, 130-138.
111. Dhar, S.; Gu, F. X.; Langer, R.; Farokhzad, O. C.; Lippard, S. J., Targeted Delivery of Cisplatin to Prostate Cancer Cells by Aptamer Functionalized Pt(Iv) Prodrug-Plga-Peg Nanoparticles. Proc. Natl. Acad. Sci. U.S.A. 2008, 105, 17356-17361.
112. Li, X.; Wang, L.-K.; Wang, L.-W.; Han, X.-Q.; Yang, F.; Gong, Z.-J., Cisplatin Protects against Acute Liver Failure by Inhibiting Nuclear Hmgb1 Release. Int. J. Mol. Sci. 2013, 14, 11224-11237.
113. Basu, A.; Krishnamurthy, S., Cellular Responses to Cisplatin-Induced DNA Damage. J. Nucleic Acids 2010, 2010.
114. Kang, T.-H.; Lindsey-Boltz, L. A.; Reardon, J. T.; Sancar, A., Circadian Control of Xpa and Excision Repair of Cisplatin-DNA Damage by Cryptochrome and Herc2 Ubiquitin Ligase. Proc. Natl. Acad. Sci. U.S.A. 2010, 107, 4890-4895.
115. Newman, M. S.; Colbern, G. T.; Working, P. K.; Engbers, C.; Amantea, M. A., Comparative Pharmacokinetics, Tissue Distribution, and Therapeutic Effectiveness of Cisplatin Encapsulated in Long-Circulating, Pegylated Liposomes (Spi-077) in Tumor-Bearing Mice. Cancer Chemoth. Pharm. 1999, 43, 1-7.
116. Comenge, J.; Sotelo, C.; Romero, F.; Gallego, O.; Barnadas, A.; Parada, T. G.-C.; Domínguez, F.; Puntes, V. F., Detoxifying Antitumoral Drugs Via Nanoconjugation: The Case of Gold Nanoparticles and Cisplatin. PLoS One 2012, 7, e47562.
117. Wang, Y.; Juan, L.; Ma, X.; Wang, D.; Ma, H.; Chang, Y.; Nie, G.; Jia, L.; Duan, X.; Liang, X.-J., Specific Hemosiderin Deposition in Spleen Induced by a Low Dose of Cisplatin: Altered Iron Metabolism and Its Implication as an Acute Hemosiderin Formation Model. Curr. Drug Metab. 2010, 11, 507-515.
118. Guo, S.; Miao, L.; Wang, Y.; Huang, L., Pure Cisplatin Nanoparticle with Tunable Size and Surface Modification for Melanoma Cancer Therapy. 2013, in submission.
119. Graf, N.; Bielenberg, D. R.; Kolishetti, N.; Muus, C.; Banyard, J.; Farokhzad, O. C.; Lippard, S. J., Alpha(V)Beta(3) Integrin-Targeted Plga-Peg Nanoparticles for Enhanced Anti-Tumor Efficacy of a Pt(Iv) Prodrug. ACS Nano 2012, 6, 4530-4539.
120. Mcgahan, M. C.; Tyczkowska, K., The Determination of Platinum in Biological-Materials by Electrothermal Atomic-Absorption Spectroscopy. Spectrochim Acta B 1987, 42, 665-668.
121. Yao, X.; Panichpisal, K.; Kurtzman, N.; Nugent, K., Cisplatin Nephrotoxicity: A Review. Am. J. Med. Sci. 2007, 334, 115-124.
122. Pabla, N.; Dong, Z., Cisplatin Nephrotoxicity: Mechanisms and Renoprotective Strategies. Kidney Int. 2008, 73, 994-1007.
123. Plummer, R.; Wilson, R. H.; Calvert, H.; Boddy, A. V.; Griffin, M.; Sludden, J.; Tilby, M. J.; Eatock, M.; Pearson, D. G.; Ottley, C. J., et al., A Phase I Clinical Study of Cisplatin-Incorporated Polymeric Micelles (Nc-6004) in Patients with Solid Tumours. Br. J. Cancer 2011, 104, 593-598.
124. Cabral, H.; Matsumoto, Y.; Mizuno, K.; Chen, Q.; Murakami, M.; Kimura, M.; Terada, Y.; Kano, M. R.; Miyazono, K.; Uesaka, M., et al., Accumulation of Sub-100 Nm Polymeric Micelles in Poorly Permeable Tumours Depends on Size. Nat. Nanotechnol. 2011, 6, 815-823.
125. Baba, M.; Matsumoto, Y.; Kashio, A.; Cabral, H.; Nishiyama, N.; Kataoka, K.; Yamasoba, T., Micellization of Cisplatin (Nc-6004) Reduces Its Ototoxicity in Guinea Pigs. J. Control. Release 2012, 157, 112-117.
126. Sengupta, P.; Basu, S.; Soni, S.; Pandey, A.; Roy, B.; Oh, M. S.; Chin, K. T.; Paraskar, A. S.; Sarangi, S.; Connor, Y., et al., Cholesterol-Tethered Platinum Ii-Based Supramolecular Nanoparticle Increases Antitumor Efficacy and Reduces Nephrotoxicity. Proc. Natl. Acad. Sci. U.S.A. 2012, 109, 11294-11299.
127. Harrington, K. J.; Rowlinson-Busza, G.; Syrigos, K. N.; Vile, R. G.; Uster, P. S.; Peters, A. M.; Stewart, J. S., Pegylated Liposome-Encapsulated Doxorubicin and Cisplatin Enhance the Effect of Radiotherapy in a Tumor Xenograft Model. Clin. Cancer. Res. 2000, 6, 4939-4949.
128. Zisman, N.; Dos Santos, N.; Johnstone, S.; Tsang, A.; Bermudes, D.; Mayer, L.; Tardi, P., Optimizing Liposomal Cisplatin Efficacy through Membrane Composition Manipulations. Chemother. Res. Pract. 2011, 2011.
129. Zamboni, W.; Gervais, A.; Egorin, M.; Schellens, J. M.; Zuhowski, E.; Pluim, D.; Joseph, E.; Hamburger, D.; Working, P.; Colbern, G., et al., Systemic and Tumor Disposition of Platinum after Administration of Cisplatin or Stealth Liposomal-Cisplatin Formulations (Spi-077 and Spi-077 B 103) in a Preclinical Tumor Model of Melanoma. Cancer Chemother. Pharmacol. 2004, 53, 329-336.
130. Vail, D. M.; Kurzman, I. D.; Glawe, P. C.; O'Brien, M. G.; Chun, R.; Garrett, L. D.; Obradovich, J. E.; Fred, R. M., 3rd; Khanna, C.; Colbern, G. T., et al., Stealth Liposome-Encapsulated Cisplatin (Spi-77) Versus Carboplatin as Adjuvant Therapy for Spontaneously Arising Osteosarcoma (Osa) in the Dog: A Randomized Multicenter Clinical Trial. Cancer Chemother. Pharmacol. 2002, 50, 131-136.
131. Seetharamu, N.; Kim, E.; Hochster, H.; Martin, F.; Muggia, F., Phase Ii Study of Liposomal Cisplatin (Spi-77) in Platinum-Sensitive Recurrences of Ovarian Cancer. Anticancer Res. 2010, 30, 541-545.
132. Khiati, S.; Luvino, D.; Oumzil, K.; Chauffert, B.; Camplo, M.; Barthelemy, P., Nucleoside-Lipid-Based Nanoparticles for Cisplatin Delivery. ACS Nano 2011, 5, 8649-8655.
133. Dhar, S.; Kolishetti, N.; Lippard, S. J.; Farokhzad, O. C., Targeted Delivery of a Cisplatin Prodrug for Safer and More Effective Prostate Cancer Therapy in Vivo. Proc. Natl. Acad. Sci. U.S.A. 2011, 108, 1850-1855.
134. Avgoustakis, K.; Beletsi, A.; Panagi, Z.; Klepetsanis, P.; Karydas, A. G.; Ithakissios, D. S., Plga-Mpeg Nanoparticles of Cisplatin: In Vitro Nanoparticle Degradation, in Vitro Drug Release and in Vivo Drug Residence in Blood Properties. J. Control. Release 2002, 79, 123-135.
135. Kolishetti, N.; Dhar, S.; Valencia, P. M.; Lin, L. Q.; Karnik, R.; Lippard, S. J.; Langer, R.; Farokhzad, 0. C., Engineering of Self-Assembled Nanoparticle Platform for Precisely Controlled Combination Drug Therapy. Proc. Natl. Acad. Sci. U.S.A. 2010, 107, 17939-17944.
136. Dhar, S.; Gu, F. X.; Langer, R.; Farokhzad, O. C.; Lippard, S. J., Targeted Delivery of Cisplatin to Prostate Cancer Cells by Aptamer Functionalized Pt(Iv) Prodrug-Plga-Peg Nanoparticles. Proc. Natl. Acad. Sci. U.S.A. 2008, 105, 17356-17361.
137. Boulikas, T., Clinical Overview on Lipoplatin: A Successful Liposomal Formulation of Cisplatin. Expert Opin. Investig. Drugs 2009, 18, 1197-1218.
138. Boulikas, T., Therapy for Human Cancers Using Cisplatin and Other Drugs or Genes Encapsulated into Liposomes. Google Patents: 2003.
139. Alderden, R. A.; Hall, M. D.; Hambley, T. W., The Discovery and Development of Cisplatin. J. Chem. Educ. 2006, 83, 728.
140. Aryal, S.; Hu, C.-M. J.; Zhang, L., Polymer—Cisplatin Conjugate Nanoparticles for Acid-Responsive Drug Delivery. ACS Nano 2009, 4, 251-258.
141. Dhar, S.; Gu, F. X.; Langer, R.; Farokhzad, O. C.; Lippard, S. J., Targeted Delivery of Cisplatin to Prostate Cancer Cells by Aptamer Functionalized Pt (Iv) Prodrug-Plga-Peg Nanoparticles. Proc. Natl. Acad. Sci. U.S.A. 2008, 105, 17356-17361.
142. Zhang, Y.; Kim, W. Y.; Huang, L., Systemic Delivery of Gemcitabine Triphosphate Via Lcp Nanoparticles for Nscic and Pancreatic Cancer Therapy. Biomaterials 2013.
143. Li, J.; Yang, Y.; Huang, L., Calcium Phosphate Nanoparticles with an Asymmetric Lipid Bilayer Coating for Sirna Delivery to the Tumor. J. Control. Release 2012, 158, 108-114.
144. Hu, Y.; Haynes, M. T.; Wang, Y.; Liu, F.; Huang, L., A Highly Efficient Synthetic Vector: Nonhydrodynamic Delivery of DNA to Hepatocyte Nuclei in Vivo. ACS Nano 2013, 7, 5376-5384.
145. Burger, K. N.; Staffhorst, R. W.; de Vijlder, H. C.; Velinova, M. J.; Bomans, P. H.; Frederik, P. M.; de Kruijff, B., Nanocapsules: Lipid-Coated Aggregates of Cisplatin with High Cytotoxicity. Nat. Med. 2002, 8, 81-84.
146. Khiati, S.; Luvino, D.; Oumzil, K.; Chauffert, B.; Camplo, M.; Barthelemy, P., Nucleoside-Lipid-Based Nanoparticles for Cisplatin Delivery. ACS Nano 2011, 5, 8649-8655.
147. Velinova, M. J.; Staffhorst, R. W.; Mulder, W. J.; Dries, A. S.; Jansen, B. A.; de Kruijff, B.; de Kroon, A. I., Preparation and Stability of Lipid-Coated Nanocapsules of Cisplatin: Anionic Phospholipid Specificity. Biochim. Biophys. Acta, Biomembr. 2004, 1663, 135-142.
148. Lin, C.-A. J.; Sperling, R. A.; Li, J. K.; Yang, T.-Y.; Li, P.-Y.; Zanella, M.; Chang, W. H.; Parak, W. J., Design of an Amphiphilic Polymer for Nanoparticle Coating and Functionalization. Small 2008, 4, 334-341.
149. Chen, T.; Öçsoy, I.; Yuan, Q.; Wang, R.; You, M.; Zhao, Z.; Song, E.; Zhang, X.; Tan, W., One-Step Facile Surface Engineering of Hydrophobic Nanocrystals with Designer Molecular Recognition. J. Am. Chem. Soc. 2012, 134, 13164-13167.
150. Nakagawa, O.; Ming, X.; Huang, L.; Juliano, R. L., Targeted Intracellular Delivery of Antisense Oligonucleotides Via Conjugation with Small-Molecule Ligands. J. Am. Chem. Soc. 2010, 132, 8848-8849.
151. Xu, Y.; Szoka, F. C., Mechanism of DNA Release from Cationic Liposome/DNA Complexes Used in Cell Transfection†,‡. Biochemistry 1996, 35, 5616-5623.
152. Banerjee, R.; Tyagi, P.; Li, S.; Huang, L., Anisamide—Targeted Stealth Liposomes: A Potent Carrier for Targeting Doxorubicin to Human Prostate Cancer Cells. Int. J. Cancer 2004, 112, 693-700.

1. A delivery system complex comprising a bioactive compound, wherein said bioactive compound is a nano-precipitated salt compound having at least a portion of its surface coated by a liposome or encapsulated by a liposome, wherein said nano-precipitated bioactive compound has low solubility in water and oil and is present in an amount of at least 10% wt of said liposome.

2. (canceled)

3. The delivery system complex of claim 1, wherein said bioactive compound is a platinum coordination complex.

4. The delivery system complex of claim 1, wherein said bioactive compound is selected from the group consisting of a cis-diaminedihaloplatinum(II) compound, a cis-diaminedichloroplatinum(II) compound, a cis-diaminedibromoplatinum(II) compound, and cis-diaminediiodoplatinum(II).

5-7. (canceled)

8. The delivery system complex of claim 1, wherein said liposome comprises a lipid bilayer having an inner leaflet and an outer leaflet, and wherein said outer leaflet comprises one or both of a cationic lipid and a lipid-poly(ethylene glycol) (lipid-PEG) conjugate.

9. The delivery system complex of claim 8, wherein said bioactive compound is ionically bound to said inner leaflet.

10. The delivery system complex of claim 8, wherein said lipid-PEG conjugate comprises PEG in an amount between about 5 mol % to about 50 mol % of total surface lipid.

11. The delivery system complex of claim 1, wherein said bioactive compound is a chemotherapeutic drug.

12. The delivery system complex of claim 11, wherein said bioactive compound has a phosphate group.

13-16. (canceled)

17. The delivery system complex of claim 1, wherein said bioactive compound is present in an amount of between 20% wt. and 70% wt of said liposome.

18-20. (canceled)

21. The delivery system complex of claim 1, wherein said delivery system complex has a diameter of less than about 100 nm.

22-26. (canceled)

27. The delivery system complex of claim 8, wherein:
said inner leaflet comprises DOPA and
said outer leaflet comprises cholesterol, DOTAP and a lipid-poly(ethylene glycol) (lipid-PEG) conjugate.

28. A method of preparing a bioactive compound nano-precipitate encapsulated by a liposome, comprising:
a. contacting a first reverse emulsion comprising a bioactive compound or a precursor thereof with a second reverse emulsion comprising a reagent that is capable of forming a species that can combine with said compound or precursor to form a nano-precipitated bioactive compound, wherein at least one of said first and second reverse emulsion further comprises a neutral or anionic lipid and;
b. allowing said nano-precipitate to form, wherein said nano-precipitate has at least a portion of its surface coated with said neutral or anionic lipid; and
c. contacting said nano-precipitate from (b) with one or more lipids to prepare a bioactive compound nano-precipitate encapsulated by a liposome.

29-31. (canceled)

32. The method of claim 28, further comprising a washing step after (b) and before (c).

33. A method of treating a cancer comprising administering the delivery system complex of claim 1 to a subject.

34. (canceled)

35. The method of claim 33, wherein said cancer is melanoma.

36. (canceled)

37. The delivery system complex of claim 1, wherein said salt is a bioactive compound complexed with a mono, di or trivalent cation.

38. (canceled)

39. The delivery system complex of claim 37, wherein said bioactive compound is selected from the group consisting of tenofovir, adefovir, acyclovir monophosphate, L-thymidine monophosphate, etoposide monophosphate, gemcitabine monophosphate, alendronate and pamidronate.

40. The delivery system complex of claim 1, wherein said salt is a bioactive compound complexed with a mono, di or trivalent anion.

41-43. (canceled)

44. The delivery system complex of claim 26 8, wherein said salt is a bioactive compound complexed with In⁺³.

45. The delivery system complex of claim 39, wherein the bioactive compound is etoposide monophosphate.
